SEQUENCE LISTING (118 sequences)

SEQ ID NO: 1 - protein, 329 aa, Bos taurus
ASTTAPKVYPLSSCCGDKSSSTVTLGCLVSSYMPEPVTVTWNSGALKSGVHTFPAVLQSSGLYSLSSMVTVPGSTSGTQTFTCNVAHPASSTKVDKAVDPRCKTTCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 2 - protein, 329 aa, Bos taurus
ASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 3 - protein, 329 aa, Bos taurus
ASTTAPKVYPLSSCCGDKSSSTVTLGCLVSSYMPEPVTVTWNSGALKSGVHTFPAVLQSSGLYSLSSMVTVPASSSGTQTFTCNVAHPASSTKVDKAVDPRCKRPCDCCPPPELPGGPSVFIFPPKPKDTLTISGTPEVTCVVVDVGHDDPEVKFSWFVDNVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 4 - protein, 25 aa, Bos taurus
DKAVDPRCKPSPCDCCPPPELPGGP

SEQ ID NO: 5 - protein, 28 aa, Bos taurus
VDKAVDPRCKTTCDCCPPPELPGGPSVF

SEQ ID NO: 6 - protein, 28 aa, Bos taurus
VDKAVDPRCKRPCDCCPPPELPGGPSVF

SEQ ID NO: 7 - protein, 29 aa, Bos taurus
VDKAVDPTCKPSPCDCCPPPELPGGPSVF

SEQ ID NO: 8 - protein, 30 aa, Ovis aries
VDKRVEPGCPDPCKHCRCPPPELPGGPSVF

SEQ ID NO: 9 - protein, 28 aa, Lama glama
VDKRVEPHGGCTCPQCPAPELPGGPSVF

SEQ ID NO: 10 - protein, 32 aa, Meriones unguiculatus
VDKTVEPRGTKHICPDCPKCPAPDLSGGPSVF

SEQ ID NO: 11 - protein, 34 aa, Felis catus
VDKTVRKTDHPPGPKPCDCPKCPPPEMLGGPSIF

SEQ ID NO: 12 - protein, 12 aa, Bos taurus
DPRCKTTCDCCP

SEQ ID NO: 13 - protein, 13 aa, Bos taurus
DPTCKPSPCDCCP

SEQ ID NO: 14 - protein, 12 aa, Bos taurus
DPRCKRPCDCCP

SEQ ID NO: 15 - protein, 98 aa, Bos taurus
ASTTAPKVYPLSSCCGDKSSSTVTLGCLVSSYMPEPVTVTWNSGALKSGVHTFPAVLQSSGLYSLSSMVTVPGSTSGTQTFTCNVAHPASSTKVDKAV

SEQ ID NO: 16 - protein, 97 aa, Bos taurus
ASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAV

SEQ ID NO: 17 - protein, 98 aa, Bos taurus
ASTTAPKVYPLSSCCGDKSSSTVTLGCLVSSYMPEPVTVTWNSGALKSGVHTFPAVLQSSGLYSLSSMVTVPASSSGTQTFTCNVAHPASSTKVDKAV

SEQ ID NO: 18 - protein, 110 aa, Bos taurus
PPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTK

SEQ ID NO: 19 - protein, 110 aa, Bos taurus
PPELPGGPSVFIFPPKPKDTLTISGTPEVTCVVVDVGHDDPEVKFSWFVDNVEVNTATTKPREEQFNSTYRVVSALRIQHQDWTGGKEFKCKVHNEGLPAPIVRTISRTK

SEQ ID NO: 20 - protein, 109 aa, Bos taurus
GPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 21 - protein, 109 aa, Bos taurus
GPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 22 - skipped

SEQ ID NO: 23 - protein, 215 aa, synthetic construct
  REGION 1..215: note = Description of Artificial Sequence: Synthetic polypeptide
QAVLGQPSSVSGSLGQRVSITCSGSSSNIGTYGVEWYQQVPGSGLRTIIYGSNSRPSGVPDRFSGSKSGNTATLTISSLQAEDEADYFCAAGDSSSRGAVFGSGTLTALGQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 24 - protein, 451 aa, synthetic construct
  REGION 1..451: note = Description of Artificial Sequence: Synthetic polypeptide
QVQLRESGPSLVKPSQTLSLTCTVSGFSLSSYALTWVRQAPGKALEWVGGITSGGTTYYNPALKSRLSITKENSKSQVSLSVSSVTPEDTATYYCARSTYGEVGDGAIADAWGQGLLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 25 - protein, 33 aa, synthetic construct
  REGION 1..33: note = Description of Artificial Sequence: Synthetic polypeptide
LQLQPFPQPQLPYPQPQLPYPQPQLPYPQPQPF

SEQ ID NO: 26 - protein, 214 aa, synthetic construct
  REGION 1..214: note = Description of Artificial Sequence: Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 226 aa, synthetic construct
  REGION 1..226: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 28 - protein, 450 aa, synthetic construct
  REGION 1..450: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 452 aa, synthetic construct
  REGION 1..452: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 30 - protein, 450 aa, synthetic construct
  REGION 1..450: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKTTCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 31 - protein, 450 aa, synthetic construct
  REGION 1..450: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKIDCSKCHNQPCVREPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 32 - protein, 474 aa, synthetic construct
  REGION 1..474: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRPVPTTPKTTIPPGKPTTQESEVEKTPCQCSKCPEPLGGLSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 33 - protein, 215 aa, synthetic construct
  REGION 1..215: note = Description of Artificial Sequence: Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 34 - protein, 213 aa, synthetic construct
  REGION 1..213: note = Description of Artificial Sequence: Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKGQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 35 - protein, 449 aa, synthetic construct
  REGION 1..449: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 36 - protein, 215 aa, synthetic construct
  REGION 1..215: note = Description of Artificial Sequence: Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRSDAEPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 37 - protein, 450 aa, synthetic construct
  REGION 1..450: note = Description of Artificial Sequence: Synthetic polypeptide
QVQLQQPGAELVKPGTSVKLSCKGYGYTFTSYWMHWVKQRPGQGLEWIGEIDPSESNTNYNQKFKGKATLTVDISSSTAYMQLSSLTSEDSAVYYCARGGYDGWDYAIDYWGQGTSVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 38 - protein, 220 aa, synthetic construct
  REGION 1..220: note = Description of Artificial Sequence: Synthetic polypeptide
DVVVTQTPLSLPVSFGDQVSISCRSSQSLAKSYGNTYLSWYLHKPGQSPQLLIYGISNRFSGVPDRFSGSGSGTDFTLKISTIKPEDLGMYYCLQGTHQPYTFGGGTKLEIKRSDAEPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 39 - protein, 448 aa, synthetic construct
  REGION 1..448: note = Description of Artificial Sequence: Synthetic polypeptide
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 40 - protein, 215 aa, synthetic construct
  REGION 1..215: note = Description of Artificial Sequence: Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRSDAEPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 41 - protein, 453 aa, synthetic construct
  REGION 1..453: note = Description of Artificial Sequence: Synthetic polypeptide
QVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 42 - protein, 220 aa, synthetic construct
  REGION 1..220: note = Description of Artificial Sequence: Synthetic polypeptide
DIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIKRSDAEPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 43 - protein, 444 aa, synthetic construct
  REGION 1..444: note = Description of Artificial Sequence: Synthetic polypeptide
EVQLQQSGPELVKPGASVKMSCKASGYTFTSNVMHWVKQKPGQGLEWIGYINPYNDGTKYNEKFKGKATLTSDKSSSTAYMELSSLTSEDSAVYYCARNWDVAYWGQGTLVTVSAASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 44 - protein, 215 aa, synthetic construct
  REGION 1..215: note = Description of Artificial Sequence: Synthetic polypeptide
DIVMTQSPATLSVTPGDRVSLSCRASQSISDYLHWYRQKSHESPRLLIKYASQSISGIPSRFSGSGSGSDFTLSINSVEPEDVGVYYCQNGHSFPFTFGSGTKLEIKRSDAEPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 45 - protein, 450 aa, synthetic construct
  REGION 1..450: note = Description of Artificial Sequence: Synthetic polypeptide
EVQLVESGGGLVQPGGSLRLSCAASGFTLSGDWIHWVRQAPGKGLEWLGEISAAGGYTDYADSVKGRFTISADTSKNTAYLQMNSLRAEDTAVYYCARESRVSFEAAMDYWGQGTLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 46 - protein, 215 aa, synthetic construct
  REGION 1..215: note = Description of Artificial Sequence: Synthetic polypeptide
DIQMTQSPSSLSASVGDRVTITCRASQDLATDVAWYQQKPGKAPKLLIYSASFLYSGVPSRFSGSGSGTDFTLTISSLQPEDFATYYCQQSEPEPYTFGQGTKVEIKRSDAEPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 47 - protein, 446 aa, synthetic construct
  REGION 1..446: note = Description of Artificial Sequence: Synthetic polypeptide
EVQLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 48 - protein, 212 aa, synthetic construct
  REGION 1..212: note = Description of Artificial Sequence: Synthetic polypeptide
QIVSTQSPAIMSASPGEKVTMTCSASSSRSYMQWYQQKPGTSPKRWIYDTSKLASGVPARFSGSGSGTSYSLTISSMEAEDAATYYCHQRSSYTFGGGTKLEIKRSDAEPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 49 - protein, 445 aa, synthetic construct
  REGION 1..445: note = Description of Artificial Sequence: Synthetic polypeptide
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 50 - protein, 215 aa, synthetic construct
  REGION 1..215: note = Description of Artificial Sequence: Synthetic polypeptide
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRSDAEPSVFLFKPSDEQLKTGTVSVVCLVNDFYPKDINVKWKVDGVTQSSSNFQNSFTDQDSKKSTYSLSSILTLPSSEYQSHNAYTCEVSHKSLTTALVKSFSKNEC

SEQ ID NO: 51 - protein, 78 aa, Homo sapiens
ASTKGPSVFPLAPSSKSTSGGTAALGCLVKDYFPEPVTVSWNSGALTSGVHTFPAVLQSSGLYSLSSVVTVPSSSLGT

SEQ ID NO: 52 - protein, 77 aa, Bos taurus
ASTTAPKVYPLSSCCGDKSSSTVTLGCLVSSYMPEPVTVTWNSGALKSGVHTFPAVLQSSGLYSLSSMVTVPGSTSG

SEQ ID NO: 53 - protein, 78 aa, synthetic construct
  REGION 1..78: note = Description of Artificial Sequence: Synthetic polypeptide
ASTKGPSVFPLSSCCGDKSGGTAALGCLVKDYFPEPVTVSWNSGALTSGVHTFPAVLQSSGLYSLSSVVTVPSSSLGT

SEQ ID NO: 54 - protein, 78 aa, Homo sapiens
QTYICNVNHKPSNTKVDKKVEPKSCDKTHTCPPCPAPELLGGPSVFLFPPKPKDTLMISRTPEVTCVVVDVSHEDPEV

SEQ ID NO: 55 - protein, 76 aa, Bos taurus
QTFTCNVAHPASSTKVDKAVDPTCKPSPCDCCPPPELPGGPSVFIFPPKPKDTLTISGTPEVTCVVVDVGHDDPEV

SEQ ID NO: 56 - protein, 76 aa, synthetic construct
  REGION 1..76: note = Description of Artificial Sequence: Synthetic polypeptide
QTYICNVNHKPSNTKVDKAVDPTCKPSPCDCCPPPELLGGPSVFLFPPKPKDTLMISRTPEVTCVVVDVSHEDPEV

SEQ ID NO: 57 - protein, 78 aa, Homo sapiens
ASTKGPSVFPLAPCSRSTSESTAALGCLVKDYFPEPVTVSWNSGALTSGVHTFPAVLQSSGLYSLSSVVTVPSSNFGT

SEQ ID NO: 58 - protein, 77 aa, Bos taurus; identical to SEQ ID NO: 52

SEQ ID NO: 59 - protein, 78 aa, synthetic construct
  REGION 1..78: note = Description of Artificial Sequence: Synthetic polypeptide
ASTKGPSVFPLSSCCGDKSESTAALGCLVKDYFPEPVTVSWNSGALTSGVHTFPAVLQSSGLYSLSSVVTVPSSNFGT

SEQ ID NO: 60 - protein, 74 aa, Homo sapiens
QTYTCNVDHKPSNTKVDKTVERKCCVECPPCPAPPVAGPSVFLFPPKPKDTLMISRTPEVTCVVVDVSHEDPEV

SEQ ID NO: 61 - protein, 76 aa, Bos taurus; identical to SEQ ID NO: 55

SEQ ID NO: 62 - protein, 76 aa, synthetic construct
  REGION 1..76: note = Description of Artificial Sequence: Synthetic polypeptide
QTYTCNVDHKPSNTKVDKAVDPTCKPSPCDCCPPPELPAGPSVFLFPPKPKDTLMISRTPEVTCVVVDVSHEDPEV

SEQ ID NO: 63 - protein, 78 aa, Homo sapiens
ASTKGPSVFPLAPCSRSTSESTAALGCLVKDYFPEPVTVSWNSGALTSGVHTFPAVLQSSGLYSLSSVVTVPSSSLGT

SEQ ID NO: 64 - protein, 77 aa, Bos taurus; identical to SEQ ID NO: 52

SEQ ID NO: 65 - protein, 78 aa, synthetic construct
  REGION 1..78: note = Description of Artificial Sequence: Synthetic polypeptide
ASTKGPSVFPLSSCCGDKSESTAALGCLVKDYFPEPVTVSWNSGALTSGVHTFPAVLQSSGLYSLSSVVTVPSSSLGT

SEQ ID NO: 66 - protein, 75 aa, Homo sapiens
KTYTCNVDHKPSNTKVDKRVESKYGPPCPSCPAPEFLGGPSVFLFPPKPKDTLMISRTPEVTCVVVDVSQEDPEV

SEQ ID NO: 67 - protein, 76 aa, Bos taurus; identical to SEQ ID NO: 55

SEQ ID NO: 68 - protein, 76 aa, synthetic construct
  REGION 1..76: note = Description of Artificial Sequence: Synthetic polypeptide
KTYTCNVDHKPSNTKVDKAVDPTCKPSPCDCCPAPEFLGGPSVFLFPPKPKDTLMISRTPEVTCVVVDVSQEDPEV

SEQ ID NO: 69 - protein, 449 aa, synthetic construct
  REGION 1..449: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTTAPKVYPLSSSCGDKSSSTVTLGCLVSSYMPEPVTVTWNSGALKSGVHTFPAVLQSSGLYSLSSMVTVPGSTSGQTFTCNVAHPASSTKVDKAVDPTSKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 70 - protein, 215 aa, synthetic construct; identical to SEQ ID NO: 36
  REGION 1..215: note = Description of Artificial Sequence: Synthetic polypeptide

SEQ ID NO: 71 - protein, 449 aa, synthetic construct
  REGION 1..449: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTTAPKVYPLSSCSGDKSSSTVTLGCLVSSYMPEPVTVTWNSGALKSGVHTFPAVLQSSGLYSLSSMVTVPGSTSGQTFTCNVAHPASSTKVDKAVDPTSKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 72 - protein, 215 aa, synthetic construct; identical to SEQ ID NO: 36
  REGION 1..215: note = Description of Artificial Sequence: Synthetic polypeptide

SEQ ID NO: 73 - protein, 449 aa, synthetic construct
  REGION 1..449: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTTAPKVYPLSSSSGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 74 - protein, 215 aa, synthetic construct; identical to SEQ ID NO: 36
  REGION 1..215: note = Description of Artificial Sequence: Synthetic polypeptide

SEQ ID NO: 75 - protein, 451 aa, synthetic construct
  REGION 1..451: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTTPPKVYPLTSCCGDTSSSIVTLGCLVSSYMPEPVTVTWNSGALTSGVHTFPAILQSSGLYSLSSVVTVPASTSGAQTFICNVAHPASSTKVDKRVEPGCPDPCKHCRCPPPELPGGPSVFIFPPKPKDTLTISGTPEVTCVVVDVGQDDPEVQFSWFVDNVEVRTARTKPREEQFNSTFRVVSALPIQHQDWTGGKEFKCKVHNEALPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 76 - protein, 214 aa, synthetic construct
  REGION 1..214: note = Description of Artificial Sequence: Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRSDAQPSVFLFKPSEEQLRTGTVSVVCLVNDFYPKDINVKVKVDGVTQNSNFQNSFTDQDSKKSTYSLSSTLTLSSSEYQSHNAYACEVSHKSLPTALVKSFNKNEC

SEQ ID NO: 77 - protein, 450 aa, synthetic construct
  REGION 1..450: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTTPPKVYPLTSCCGDTSSSIVTLGCLVSSYMPEPVTVTWNSGALTSGVHTFPAVLQSSGLYSLSSMVTVPASTSGAQTFICNVAHPASSTKVDKRVEPGCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 78 - protein, 212 aa, synthetic construct
  REGION 1..212: note = Description of Artificial Sequence: Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKGQPKSAPSVTLFPPSTEELNANKATVVCLISDFYPGSVTVVWKADGSTINQNVKTTQASKQSNSKYAASSYLTLTGSEWKSKSSYSCEVTHEGSTVKKTVKPSEC

SEQ ID NO: 79 - protein, 443 aa, synthetic construct
  REGION 1..443: note = Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSGSFKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 80 - protein, 211 aa, synthetic construct
  REGION 1..211: note = Description of Artificial Sequence: Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKGDPVAPTVLIFPPAADQVATGTVTIVCVANKYFPDVTVTWEVDGTTQTTGIENSKTPQDSADCTYNLSSTLTLTSTQYNSHKEYTCKVTQGTTSVVQSFNRGDC

SEQ ID NO: 81 - protein, 57 aa, Homo sapiens
CPPCPAPELLGGPSVFLFPPKPKDTLMISRTPEVTCVVVDVSHEDVSNKALPAPIEK

SEQ ID NO: 82 - protein, 57 aa, Bos taurus
CDCCPPPELPGGPSVFIFPPKPKDTLTISGTPEVTCVVVDVGHDDVHNEGLPAPIVR

SEQ ID NO: 83 - protein, 57 aa, synthetic construct
  REGION 1..57: note = Description of Artificial Sequence: Synthetic polypeptide
CDCCPPPELLGGPSVFIFPPKPKDTLTISGTPEVTCVVVDVSHDDVHNEALPAPIVR

SEQ ID NO: 84 - protein, 51 aa, synthetic construct
  REGION 1..51: note = Description of Artificial Sequence: Synthetic polypeptide
VDKAVTARRPVPTTPKTTIPPGKPTTPKSEVEKTPCQCSKCPEPLGGLSVF

SEQ ID NO: 85 - protein, 29 aa, synthetic construct
  REGION 1..29: note = Description of Artificial Sequence: Synthetic peptide
VDKAVDPRCKPSPCDCCPPPELPGGPSVF

SEQ ID NO: 86 - protein, 55 aa, synthetic construct
  REGION 1..55: note = Description of Artificial Sequence: Synthetic polypeptide
VDKAVTARRPVPTTPKTTIPPGKPTTPKSEVEPRCKPSPCDCCPPPELPGGPSVF

SEQ ID NO: 87 - protein, 26 aa, Bos taurus
AVDPTCKPSPCDCCPPPELPGGPSVF

SEQ ID NO: 88 - protein, 21 aa, synthetic construct
  REGION 1..21: note = Description of Artificial Sequence: Synthetic peptide
AVDPTCKPSPCDCCVREPSVF

SEQ ID NO: 89 - protein, 26 aa, synthetic construct
  REGION 1..26: note = Description of Artificial Sequence: Synthetic peptide
AVDPTCKPSPCDCCPPPPPPPPPSVF

SEQ ID NO: 90 - protein, 26 aa, synthetic construct
  REGION 1..26: note = Description of Artificial Sequence: Synthetic peptide
AVDPTCKPSPCDCCPPPGSGSGPSVF

SEQ ID NO: 91 - protein, 24 aa, Bos taurus
DKAVDPRCKTTCDCCPPPELPGGP

SEQ ID NO: 92 - protein, 28 aa, Vicugna pacos
VDKRVEPHGGCTCPQCPAPELPGGPSVF

SEQ ID NO: 93 - protein, 28 aa, Camelus dromedarius
VDKRVEPHGGCTCPQCPAPELPGGPSVF

SEQ ID NO: 94 - protein, 27 aa, Oryctolagus cuniculus
VDKTVAPSTCSKPTCPPPELLGGPSVF

SEQ ID NO: 95 - protein, 53 aa, Bos taurus
VTVPGSTSGQTFTCNVAHPASSTKVDKAVDPTCKPSPCDCCPPPELPGGPSVF

SEQ ID NO: 96 - protein, 50 aa, Bos taurus
VTVPGSTSGQTFTCNVAHPASSTKVDKAVGVSSDCSKPNNQHCVREPSVF

SEQ ID NO: 97 - protein, 50 aa, Bos taurus
VTVPASSSGQTFTCNVAHPASSTKVDKAVGVSIDCSKCHNQPCVREPSVF

SEQ ID NO: 98 - protein, 53 aa, Homo sapiens
PSSSLGTQTYICNVNHKPSNTKVDKKVEPKSCDKTHTCPPCPAPELLGGPSVF

SEQ ID NO: 99 - protein, 50 aa, Bos taurus
PGSTSGTQTFTCNVAHPASSTKVDKAVDPRCKTTCDCCPPPELPGGPSVF

SEQ ID NO: 100 - protein, 19 aa, Bos taurus
SPPSVTLFPPSTEELNGNK

SEQ ID NO: 101 - protein, 15 aa, Bos taurus
YAASSYLSLTSSDWK

SEQ ID NO: 102 - protein, 29 aa, Bos taurus
VSITCSGSSSNVGNGYVSWYQLIPGSAPR

SEQ ID NO: 103 - protein, 16 aa, Bos taurus
GSYSCEVTHEGSTVTK

SEQ ID NO: 104 - protein, 11 aa, Bos taurus
VYPLSSCCGDK

SEQ ID NO: 105 - protein, 29 aa, Bos taurus
SSSTVTLGCLVSSYMPEPVTVTWNSGALK

SEQ ID NO: 106 - protein, 45 aa, Bos taurus
SGVHTFPAVLQSSGLYSLSSMVTVPGSTSGQTFTCNVAHPASSTK

SEQ ID NO: 107 - protein, 33 aa, Bos taurus
AVDPTCKPSPCDCCPPPELPGGPSVFIFPPKPK

SEQ ID NO: 108 - protein, 25 aa, Bos taurus
DTLTISGTPEVTCVVDVGHDDPEVK

SEQ ID NO: 109 - protein, 18 aa, Bos taurus
FSWFVDDVEVNTATTKPR

SEQ ID NO: 110 - protein, 12 aa, Bos taurus
VHNEGLPAPIVR

SEQ ID NO: 111 - protein, 16 aa, Bos taurus
EPQVYVLAPPQEELSK

SEQ ID NO: 112 - protein, 23 aa, Bos taurus
STVSLTCMVTSFYPDYIAVEWQR

SEQ ID NO: 113 - protein, 28 aa, Bos taurus
NGQPESEDKYGTTPPQLDADSSYFLYSK

SEQ ID NO: 114 - protein, 16 aa, Bos taurus
EPQVYVLDPPKEELSK

SEQ ID NO: 115 - protein, 10 aa, Bos taurus
TTPPQLDADR

SEQ ID NO: 116 - protein, 12 aa, Bos taurus
EPSVFIFPPKPK

SEQ ID NO: 117 - protein, 16 aa, Bos taurus
VVSALPIQHQDWTGGK

SEQ ID NO: 118 - protein, 20 aa, Bos taurus
GDTYTCVVMHEALHNHYMQK

(19) United States
(12) Patent Application Publication
(10) Pub. No.: US 2024/0067715 A1
(43) Pub. Date: Feb. 29, 2024

(54) ANTIBODIES WITH IMPROVED STABILITY TO INTESTINAL DIGESTION, POLYNUCLEOTIDES THEREOF AND METHODS OF USE THEREOF TO TREAT DISEASE

(71) Applicant: CIRCLE33 LLC, Jackson, WY (US)

(72) Inventor: Randall Edward BURTON, Billerica, MA (US)

(21) Appl. No.: 18/344,311

(22) Filed: Jun. 29, 2023

Related U.S. Application Data
(63) Continuation of application No. 15/765,958, filed on Sep. 24, 2020.
(60) Provisional application No. 62/237,270, filed on Oct. 5, 2015.

(51) Int. Cl. C07K 16/24; A61K 9/00; A61P 1/04

(52) U.S. Cl. CPC C07K 16/241; A61K 9/0053; A61P 1/04; A61K 2039/505

(57) ABSTRACT

The present invention provides for recombinant antibodies having the features of ruminant early colostrum antibodies that impart resistance to proteases and intestinal digestion. It is a feature of the present invention that when administered to animals including humans, pharmaceutical compositions comprising the novel recombinant antibodies of the present invention, advantageously exhibit resistance to proteases and intestinal digestion. Thus, pharmaceutical compositions of the recombinant antibodies of the invention may be used to deliver antibody therapeutics particularly by oral delivery to the gastrointestinal tract when oral delivery is advantageous.

### BACKGROUND

Orally administered bovine colostral antibodies have been used experimentally to treat and prevent infection and inflammation in the digestive tract (Jones et al., Crit Rev Biotechnol 2015; July 15: 1-15). In contrast to other types of antibodies, which are readily digested, bovine colostral antibodies naturally resist intestinal digestion, making them uniquely suited for oral, topical use in the digestive tract. Studies of bovine colostral antibodies have consistently shown that they survive in the human digestive tract (Jaison et al. Nutrition Journal 2015; 14:1-8). However, studies of digestion resistance for bovine antibodies isolated from serum have been inconsistent (Jaison et al., supra; Kuribayashi et al., Comparative Medicine 2009; 59:163-7), suggesting that bovine serum antibodies lack the digestion resistance of colostral antibodies. Despite the experimental success of colostral antibodies as orally administered drugs, they are naturally produced polyclonals and therefore cannot take advantage of routine methods used to design and manufacture recombinant monoclonal antibodies as drugs.

Bovine colostrum contains a mixture of immunoglobulins including IgG1, IgG2, IgM, and IgA. In addition to proteins, colostrum contains large quantities of lipids, sugars, and small molecules. In short, bovine colostrum is a complex mixture of bioactive substances, which makes it very difficult to elucidate the relevant factors that confer digestion resistance.

Immunoglobulins in bovine colostrum are transferred from the serum to the udder shortly before parturition. Most transfer of IgG occurs by active transport via mammary epithelial cells, which secrete substances into the colostrum. During transport through these cells, IgG can be modified by glycosylation or other means. This process adds to the complexity of efforts to identify factors that confer digestion resistance to bovine colostral antibodies. Further complicating these efforts are contradictory results of previous studies that assessed the protease resistance of bovine antibodies.

Numerous studies, dating back to 1997, have shown that different bovine antibody isotypes show distinct patterns of resistance to certain proteases, with some isotypes exhibiting greater resistance to certain proteases than others, but with inconsistent and contradicting results (Butler et al., Biochim Biophys Acta 1978; 535:125-37; Brock et al., Annales de Recherches Veterinaires 1978; 9:287-94). A study by de Rham et al. (Int Archs Allergy Appl Immun 1997; 55:61-9) showed no differences in protease resistance to trypsin or pepsin between colostral and serum IgG1. In contrast, later studies found profound differences in digestion resistance between bovine colostral and serum antibodies, with colostral antibodies being more resistant to protease digestion than serum antibodies (Jaison et al., supra; Kuribayashi et al., supra). These results suggest that another factor associated only with colostral antibodies must be responsible for digestion resistance. Over time, two main hypotheses emerged to explain the apparent protease resistance of bovine colostral antibodies.

One hypothesis was that a factor present in colostrum inhibited proteolysis. For instance, Piniero et al. (Ann Rech Vet 1978:9:287-94) isolated a trypsin inhibitor from bovine colostrum, and Quigley et al. (J Dairy Sci 1995; 78:1573-7) found that the amount of this trypsin inhibitor roughly correlated with the amount of immunoglobulin in the colostrum. The presence of this trypsin inhibitor, and possibly other protease inhibitors in the colostrum, could be responsible for the digestion resistance of bovine colostral antibodies.

Another hypothesis was that glycosylation specific to bovine colostral antibodies protected them from proteases. In one study, O-linked glycosylation associated with a jacalin-binding fraction of bovine colostral immunoglobulin resisted pepsin digestion, as compared with the fraction that did not bind jacalin (Porto et al., J Dairy Sci 2007; 90:955-62). Moreover, U.S. Pat. No. 8,647,626 also discloses glycosylation as conferring stability to bovine antibodies.

Collectively, studies to date point to the lack of a definitive understanding of the factors and/or molecular basis underlying the protease resistance of bovine colostral antibodies. Elucidating these determinants would enable the manufacture of uniform, digestion resistant, orally administratable, recombinant monoclonal antibody compositions, thereby addressing an unmet, longstanding need in the field of immunotherapy.

### SUMMARY

The present invention is based, as least in part, on the identification of the structural features of ruminant early colostrum antibodies that impart resistance to proteases and intestinal digestion. When administered to animals, including humans, pharmaceutical compositions comprising recombinant antibodies as described herein advantageously exhibit resistance to proteases and intestinal digestion. Thus, these pharmaceutical compositions may be delivered to the gastrointestinal tract, particularly for the treatment of diseases of the digestive tract.

In one embodiment, the recombinant antibody retains at least the antigen binding CDRs or variable region (VR) from a parent (non-bovine) antibody, and includes all or a portion of a bovine IgG1 constant region (e.g., a bovine IgG1 CH1 and/or hinge domain). Such antibodies are referred to herein as “bovinized antibodies.”

In another embodiment, the antibody comprises the variable region of a parent (non-bovine) antibody, and the constant region (i.e., CH1 domain, hinge region, CH2 domain, and CH3 domain) of a bovine IgG1 antibody. Such antibodies are referred to herein as “bovine chimeric antibodies.”

In one aspect, provided herein are isolated recombinant monoclonal antibodies comprising (a) heavy and light chain CDR regions from a non-bovine antibody and (b) a constant region comprising all or a portion of a bovine IgG1 constant region (e.g., a bovine IgG1 constant region selected from any of SEQ ID NOs: 1-3). In one embodiment, the non-bovine antibody is a human antibody.

In one embodiment, the constant region comprises all or a portion of a bovine IgG1 hinge region. In another embodiment, the constant region comprises all or a portion of a bovine IgG1 CH1 domain. In another embodiment, the constant region comprises all or a portion of a bovine IgG1 CH2 domain. In another embodiment, the constant region comprises all or a portion of a bovine IgG1 CH3 domain. In another embodiment, the constant region further comprises a portion of a human constant region (e.g., a human IgG1 or IgG2 constant region).

In one aspect, provided herein are bovinized antibodies that incorporate one or more of the following structural features found in bovine IgG1 that impart protease resistance to the antibody:
1) a disulfide bond linking the N-terminus of the CH1 domain to the N-terminus of the hinge domain (“bovine CH1-hinge disulfide linkage”),
2) a disulfide bond linking the N-terminus of the CH1 domain to the C-terminus of the light chain (“bovine CH1-light chain disulfide linkage”),
3) a cluster of three disulfide bonds linking the hinge regions of two heavy chains together (bovine heavy chain-heavy chain disulfide linkage”), and/or
4) a hinge sequence that is less susceptible to common proteases (“bovinized hinge”).

In one embodiment, the parent (non-bovine) antibody comprises a human IgG hinge, wherein sites within the hinge which are susceptible to enzymatic or spontaneous proteolysis are replaced with the equivalent bovine hinge region sequences. In another embodiment, the parent (non-bovine) antibody comprises a bovinized hinge region, in which some or all of the amino acid residues of the hinge region are replaced with the equivalent portion of the bovine IgG1 hinge region sequence.

In another aspect, provided herein are isolated recombinant monoclonal human antibodies, wherein the constant region of the antibodies comprise one or more of the following features found in bovine IgG1:
a) a disulfide bond linking the N-terminus of the CH1 domain to the N-terminus of the hinge region,
b) a disulfide bond linking the N-terminus of the CH1 domain to the C-terminus of the light chain, and
c) a cluster of three disulfide bonds linking the hinge regions of two heavy chains together.
   In other words, the antibody has fully human sequences, except for one or more of these structural features that exist in bovine IgG1 (or structurally equivalent features of IgG1 from other ruminants or rabbit) which confer protease resistance.

In some embodiments, the antibodies described herein comprise a constant region comprising one or more of the following substitutions (Kabat numbering): threonine at position 252 is substituted with methionine; glycine at position 255 is substituted with arginine; glutamine at position 309 is substituted with leucine; threonine at position 314 is substituted with leucine; and glycine at position 315 is substituted with asparagine.

In some embodiments, the bovinized or fully bovine recombinant IgG1 monoclonal antibodies described herein bind to a biological antigen (e.g., TNF-α). In one embodiment, the antibody comprises the heavy and light chain variable region sequences of infliximab.

Bovinized antibodies described herein exhibit greater protease resistance relative to the parent antibody. In some embodiments, the bovinized antibodies also retain greater antigen-binding activity after protease digestion relative to the parent antibody. In some embodiments, the bovinized antibodies, upon being subjected to protease digestion in the GI tract, retain antigen-binding activity.

In another aspect, provided herein are fully bovine recombinant IgG1 monoclonal antibodies, as well as host cells that secrete the antibodies.

In another aspect, provided herein are bispecific bovinized antibodies or bispecific fully bovine recombinant IgG1 antibodies.

In another aspect, provided herein are immunoconjugates comprising the bovinized antibodies or fully bovine recombinant IgG1 monoclonal antibodies.

In another aspect, provided herein are nucleic acid molecules that encode the recombinant antibodies described herein. In another aspect, provided herein are expression vectors that comprise the nucleic acids. In yet another aspect, provided herein are host cells comprising the nucleic acids or expression vectors.

In another aspect, provided herein are pharmaceutical compositions containing (e.g., comprising, consisting of or consisting essentially of) the bovinized antibody or fully bovine recombinant IgG1 monoclonal antibodies or bovinized antibodies, and optionally a carrier (e.g., a pharmaceutically acceptable carrier), formulated for oral administration. In some embodiments, the composition further comprises a preservative. In some embodiments, the composition is lyophilized.

In another aspect, provided herein are methods for treating diseases (especially those involving the digestive tract) comprising administering the bovinized antibodies or fully bovine recombinant IgG1 monoclonal antibodies to a subject in need thereof. In one embodiment, the disease is ulcerative colitis. In another embodiment, the disease is Crohn's disease. In some embodiments, the antibodies bind specifically to TNF-α.

In another aspect, provided herein are kits comprising the bovinized antibodies or fully bovine recombinant IgG1 monoclonal antibodies described herein.

In another aspect, provided herein are methods of producing the bovinized antibodies or fully bovine recombinant IgG1 monoclonal antibodies described herein comprising expressing the antibodies in a host cell and isolating the antibodies from the cells.

### BRIEF DESCRIPTION OF THE FIGURES

FIG. 1 is a reducing SDS-PAGE analysis of AVX-470 subjected to pancreatin digestion under conditions discussed in Example 2.

FIG. 2 is a reducing SDS-PAGE analysis of infliximab subjected to pancreatin digestion under conditions discussed in Example 2.

FIG. 3 is a reducing SDS-PAGE analysis of human IgG1 subjected to pancreatin digestion under conditions discussed in Example 2.

FIG. 4 is a line graph plotting the pancreatin-mediated degradation of the antibody heavy chain of AVX-470, infliximab, and human IgG1, as shown in FIGS. 1-3

FIG. 5 is a line graph plotting the TNF-binding activity of AVX-470 and infliximab after pancreatin digestion for the indicated durations.

FIG. 6 is a SDS-PAGE analysis of purified colostral and serum IgG.

FIG. 7 is a SDS-PAGE analysis of serum IgG and milk IgG subjected to pancreatin digestion for the indicated durations.

FIG. 8 is a SDS-PAGE analysis of colostral IgG subjected to pancreatin digestion for the indicated durations.

FIG. 9 is a line graph plotting the pancreatin-mediated degradation of the antibody heavy chain of serum IgG, colostral IgG, and milk IgG.

FIG. 10 is a chromatogram comparing the glycosylation patterns of AVX-470 and human IgG.

FIG. 11 is a chromatogram showing the glycosylation pattern of AVX-470, and assigning 20 oligosaccharide species to the major peaks.

FIG. 12 is a chromatogram comparing the glycosylation patterns of AVX-470, cow serum IgG, and calf serum IgG.

FIG. 13 is a chromatogram comparing the glycosylation patterns of serum IgG (Dec2 bleed, Dec18 bleed) and colostral IgG (M1, M2, and M3) samples.

FIG. 14 is a chromatogram comparing the glycosylation patterns of serum IgG, colostral IgG, and milk IgG.

FIG. 15 is a line graph plotting the pancreatin-mediated degradation of the antibody heavy chain of colostral IgG treated with or without neuraminidase.

FIG. 16 is a reducing SDS-PAGE analysis of pancreatin digestion of serum IgG and colostral IgG, assigning antibody fragments to the major bands based on molecular weight of the bands and LC/MS/MS analysis.

FIG. 17 is a chromatogram showing the IgG1-enriched fraction from colostrum samples.

FIG. 18 is a chromatogram showing the IgG2-enriched fraction from serum samples.

FIG. 19 is a line graph plotting the pancreatin-mediated degradation of the antibody heavy chain of AVX-470, IgG1, and IgG2 antibodies.

FIG. 20 is a chromatogram comparing glycosylation patterns of IgG1 and IgG2 from serum.

FIG. 21 is a chromatogram comparing glycosylation patterns of IgG1 and IgG2 from colostrum.

FIG. 22 is a schematic comparing the CH1-hinge region of bovine IgG1 and IgG2.

FIG. 23 is a schematic comparing the CH1-hinge region of human IgG1 and bovine IgG1. Potential enzymatic and spontaneous proteolysis sites in human IgG1 are indicated.

FIG. 24 is a line graph comparing antigen-binding activity of bovinized infliximab, infliximab, AVX-470, and a bovine anti-testosterone monoclonal antibody, as determined by ELISA.

FIG. 25 is a Western blot analysis of anti-testosterone antibody digested with pancreatin (probed with anti-bovine IgG1 antibody).

FIG. 26 is a line graph showing titers of TNF-binding antibodies from pooled sera collected from calves immunized with the indicated adjuvants, as determined by ELISA.

FIG. 27 is a bar graph showing titers of TNF-binding antibodies from pooled sera collected from calves of the indicated groups, as determined by ELISA.

FIG. 28 is a line graph showing the TNF neutralizing activity of pooled sera collected from calves of the indicated groups, as determined by the L929 cell-based assay.

FIG. 29 is a schematic of bovinized constructs using infliximab as the parent antibody.

FIG. 30 is a line graph plotting the pancreatin-mediated degradation of the antibody heavy chain of infliximab, minimally bovinized infliximab, and bovine colostral IgG1.

FIG. 31 is schematic representation of the predicted disulfide bond structure of wild-type bovine IgG1 and the C127S/C215S variant.

FIG. 32 is schematic representation of the predicted disulfide bond structure of wild-type bovine IgG1 and the C128S/C215S variant.

FIG. 33 is schematic representation of the predicted disulfide bond structure of wild-type bovine IgG1 and the C127S/C128S variant.

FIG. 34 is a line graph plotting the pancreatin-mediated degradation of the antibody heavy chain of infliximab, bovine colostral IgG1, wild-type bovinized infliximab, and bovinized infliximab with the C127S/C215S, C128S/C215S or C127S/C128S substitutions.

FIG. 35 is a bar graph showing the levels of expression of infliximab, bovinized infliximab, and bovine anti-testosterone antibody.

### DETAILED DESCRIPTION

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well-known and commonly employed in the art. Standard techniques are used for nucleic acid and peptide synthesis. The techniques and procedures are generally performed according to conventional methods in the art and various general references (e.g., Sambrook et al., 1989, Molecular Cloning: A Laboratory Manual, 2d ed. Cold Spring Harbor Laboratory Press, Cold Spring Harbor, N.Y.), which are provided throughout this document. The nomenclature used herein and the laboratory procedures used in analytical chemistry and organic syntheses described below are those well-known and commonly employed in the art. Standard techniques or modifications thereof are used for chemical syntheses and chemical analyses.

The articles “a” and “an” are used herein to refer to one or to more than one (i.e. to at least one) of the grammatical object of the article. By way of example, “an element” means one element or more than one element.

As used herein, the term “antibody” is referred to in the broadest sense and encompasses monoclonal antibodies (including full length monoclonal antibodies), polyclonal antibodies, multispecific antibodies (e.g., bispecific antibodies), and antibody fragments including antigen binding fragments. As used herein “antibody fragments” include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site, (viii) bispecific single chain Fv dimers and (ix) “diabodies” or “triabodies”, multivalent or multispecific fragments constructed by gene fusion. The antibody fragments may be modified. For example, the molecules may be stabilized by the incorporation of disulphide bridges linking the VH and VL domains.

Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies and monoclonal antibodies. The use of the singular terms “a” or “an” or “the” antibody are not meant to be limited to a single antibody when it is clear that more than one antibody is present in the composition or preparation. In addition, unless indicated otherwise, the singular term for “antibody” may include a collection of antibodies that are not necessarily heterogenous in their structures or specificities unless indicated otherwise.

Antibodies are generally glycoproteins comprising at least two heavy (H) chains and two light (L) chains inter-connected by disulfide bonds, i.e., covalent heterotetramers comprised of two identical Ig H chains and two identical L chains that are encoded by different genes. Each heavy chain is comprised of a heavy chain variable region (abbreviated herein as HCVR or V_{H}) and a heavy chain constant region. The heavy chain constant region of an IgG subclass of immunoglobulins, for example, is comprised of three domains, CH1, CH2 and CH3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

The variable regions of the heavy and light chains contain a binding domain that interacts with an antigen. The constant regions of the antibodies may mediate the binding of the immunoglobulin to host tissues or factors, including various cells of the immune system (e.g., effector cells). Formation of a mature functional antibody molecule can be accomplished when two proteins are expressed in stoichiometric quantities and self-assemble with the proper configuration.

As used herein, the term “monoclonal antibody” refers to an antibody that displays a single binding specificity and affinity for a particular epitope or a composition of antibodies in which all antibodies display a single binding specificity and affinity for a particular epitope.

As used herein, an “isolated antibody” refers to an antibody that is substantially free of other antibodies having different antigenic specificities.

As used herein, “isotype” refers to the antibody class (e.g., IgG1, IgG2, IgG3, IgG4, IgM, IgA1, IgA2, IgD, and IgE antibody) that is encoded by the heavy chain constant region genes. In a preferred embodiment, the recombinant antibody of the invention antibody is an IgG antibody, e.g. IgG1, IgG2. In another preferred embodiment the recombinant antibody of the invention is an IgG1 antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains CH2 and CH3 and the hinge between CH1 and CH2.

As used herein, the “hinge region” of an antibody refers to a stretch of peptide sequence between the CH1 and CH2 domains of an antibody. Hinge regions occur between Fab and Fc portions of an antibody. Hinge regions are generally encoded by unique exons, and contain disulfide bonds that link the two heavy chain fragments of the antibody. The amino acid sequence of a hinge region can be generally rich in proline, serine, and threonine residues. For example, the extended peptide sequences between the CH1 and CH2 domains of IgG, IgD, and IgA are rich in prolines. IgM and IgE antibodies include a domain of about 110 amino acids that possesses hinge-like features and are included in the term “hinge region” as used herein.

As used herein, the term “region equivalent to the hinge region of a ruminant antibody” is intended to include naturally occurring allelic variants of the hinge region of a ruminant (e.g., bovine) immunoglobulin of any isotype as well as variants having alterations which produce substitutions, additions, or deletions but which do not decrease substantially the ability of the immunoglobulin to resist enzyme digestion. Such variants can be selected according to general rules known in the art so as to have minimal effect on activity.

A CH1 domain is an immunoglobulin heavy chain constant region domain. Preferred are ruminant immunoglobulin CH1 domains such as a bovine, ovine or caprine with the bovine CH1 domains being preferred. The amino acid sequence of immunoglobulin CH1 domains of various species are known or are generally available to the skilled artisan (Kabat et al., Sequences of proteins of immunological interest Fifth Ed., U.S. Department of Health and Human Services, NIH Publication No. 91-3242). In a preferred embodiment, the CH1 domain is a bovine IgG1 CH1 domain. In further preferred embodiment, the immunoglobulin CH1 domain is of the d allotype.

As used herein, the term “region equivalent to CH1 domain of a ruminant antibody” is intended to include naturally occurring allelic variants of the CH1 domain of a ruminant (e.g., bovine) immunoglobulin of any isotype as well as variants having alterations which produce substitutions, additions, or deletions but which do not decrease substantially the ability of the immunoglobulin to resist enzyme digestion. Such variants can be selected according to general rules known in the art so as to have minimal effect on activity.

A CH2 domain is an immunoglobulin heavy chain constant region domain. According to the present invention, the CH2 domain is preferably the CH2 domain of one of the five immunoglobulins subtypes indicated above. Preferred are ruminant immunoglobulin CH2 domains such as a bovine, ovine or caprine with the bovine CH2 domains being preferred. The amino acid sequence of immunoglobulin CH2 domains of various species are known or are generally available to the skilled artisan (Kabat et al., Sequences of proteins of immunological interest Fifth Ed., U.S. Department of Health and Human Services, NIH Publication No. 91-3242). A preferred immunoglobulin CH2 domain within the context of the present invention is a bovine IgG and preferably from bovine IgG1, or IgG2, and more preferably a bovine IgG1. With respect to ruminant antibody molecules reference is made to the IgG class in which an N-linked oligosaccharide is attached to the amide side chain of Asn of the inner face of the CH2 domain of the Fc region. This site is equivalent to Asn 297 of the human IgG1 immunoglobulin molecule. It is characteristic of the recombinant antibody of the present invention that it contain or be modified to contain at least a CH2 domain.

As used herein, the term “region equivalent to CH2 domain of a ruminant antibody” is intended to include naturally occurring allelic variants of the CH2 domain of a ruminant (e.g., bovine) immunoglobulin of any isotype as well as variants having alterations which produce substitutions, additions, or deletions but which do not decrease substantially the ability of the immunoglobulin to resist enzyme digestion. Such variants can be selected according to general rules known in the art so as to have minimal effect on activity.

A CH3 domain is an immunoglobulin heavy chain constant region domain. According to the present invention, the CH3 domain is preferably the CH3 domain of one of the five immunoglobulins subtypes indicated above. Preferred are ruminant immunoglobulin CH3 domains such as a bovine, ovine or caprine with the bovine CH3 domains being preferred. The amino acid sequence of immunoglobulin CH3 domains of various species are known or are generally available to the skilled artisan (Kabat et al., Sequences of proteins of immunological interest Fifth Ed., U.S. Department of Health and Human Services, NIH Publication No. 91-3242). A preferred immunoglobulin CH3 domain within the context of the present invention is from bovine IgG CH3 domain, and preferably a bovine IgG1 CH3 domain or bovine IgG2 CH3 domain, and more preferably a bovine IgG1 CH3 domain.

As used herein, the term “region equivalent to CH3 domain of a ruminant antibody” is intended to include naturally occurring allelic variants of the CH3 domain of a ruminant (e.g., bovine) immunoglobulin of any isotype as well as variants having alterations which produce substitutions, additions, or deletions but which do not decrease substantially the ability of the immunoglobulin to resist enzyme digestion. Such variants can be selected according to general rules known in the art so as to have minimal effect on activity.

As used herein the term region equivalent to the Fc region of a ruminant antibody is intended to include naturally occurring allelic variants of the Fc region of a ruminant immunoglobulin of any isotype as well as variants having alterations which produce substitutions, additions, or deletions but which do not decrease substantially the ability of the immunoglobulin to resist enzyme digestion. Such variants can be selected according to general rules known in the art so as to have minimal effect on activity.

As used herein, the term “host cell” covers any kind of cellular system which can be engineered to generate the antibodies disclosed herein Host cells include cultured cells, e.g., mammalian cultured cells, such as CHO cells, HEK293T cells, BHK cells, NS0 cells, SP2/0 cells, YO myeloma cells, P3X63 mouse myeloma cells, PER cells, PER.C6 cells or hybridoma cells, yeast cells, fungal cells, and insect cells, to name only a few, but also cells comprised within a transgenic animal or cultured tissue. In some embodiments, host cells are ruminant mammary epithelial cells including but not limited to: ruminant mammary epithelial cell lines such as Bovine cell lines BMEC+H (Bovine Mammary Epithelial Cells of the Hormone-adapted), HH2A (spontaneously immortalized bovine mammary epithelial cell line), ET-C (epithelial and myoepithelial-like characteristics) and Mac-T (Mammary Alveolar Cells).

An “isolated antibody” or “isolated antibodies”, as used herein, is intended to refer to an antibody which is substantially free of other antibodies having different antigenic specificities. An isolated antibody may be substantially free of other cellular material and/or chemicals. The term “substantially” in any of the definitions herein generally means at least about 60%, at least about 70%, at least about 80%, or more preferably at least about 90%, and still more preferably at least about 95%. Isolated also means separation from contaminant components of the antibodies' natural environment that would typically interfere with diagnostic or therapeutic uses of the antibody, such as enzymes, hormones, and other materials. As is apparent to those of skill in the art, a non-naturally occurring antibody, does not require “isolation” to distinguish it from its naturally occurring counterpart. In addition, a “concentrated”, “separated” or “diluted” antibody is distinguishable from its naturally occurring counterpart in that the concentration or number of molecules per volume is generally greater than that of its naturally occurring counterpart. In general, an antibody made by recombinant means and expressed in a host cell is considered to be “isolated.”

As used herein, “specific binding” refers to antibody binding to a predetermined epitope, isoform or variant of an antigen. Typically, the antibody binds with an affinity that is at least two-fold greater than its affinity for binding to a non-specific antigen other than the predetermined antigen or a closely-related antigen. Therefore, the antibodies provided herein in some embodiments specifically bind a target antigen.

By “affinity” or “binding affinity” as used herein is meant the strength of interaction between an antibody and its target antigen. The strength of affinity is often reported with a equilibrium dissociation constant, K_{D}, which is obtained from the ratio of k_{d} to kₐ (i.e,. k_{d}/kₐ) and is expressed as a molar concentration (M). K_{D} values for antibodies can be determined using methods well established in the art (e.g., Biacore surface Plasmon resonance, flow cytometry, Scatchard analysis). Lower values of K_{D} correspond to tighter binding and higher affinity. Higher values of K_{D} correspond to weaker binding and lower affinity.

As used herein, “target antigen” refers to the molecule that is bound specifically by the variable region of a given antibody. A target antigen may be a protein, carbohydrate, lipid, or other chemical compound.

As used herein the term “bovine chimeric antibody” or “bovinized antibody” refers to a recombinant antibody that retains at least the antigen binding CDRs or variable region (VR) from a parent (non-bovine) antibody, and includes at least a portion of a bovine IgG1 constant region (e.g., a bovine IgG1 CH1 and/or hinge domain). For example, in one embodiment, a bovinized antibody comprises the variable regions and CH2 and CH3 domains of a parent antibody (e.g., a human antibody), and the hinge region and CH1 domain of a bovine IgG1 antibody. In another embodiment, a bovinized antibody comprises one or more amino acid residues in the parent antibody that are replaced with the equivalent residue(s) of a bovine constant region, e.g., a bovine IgG1 constant region. In yet another embodiment, the bovinized antibody comprises the variable region of a parent antibody, and the CH1 domain, hinge region, CH2 domain, and CH3 domain of a bovine IgG1 antibody (i.e., a “bovine chimeric antibody”).

A “parent antibody” is used in the broadest sense and specifically covers monoclonal antibodies (including full length monoclonal antibodies), polyclonal antibodies, multispecific antibodies (e.g., bispecific antibodies), and antibody fragments including antigen binding fragments such as an Fc-fusion protein wherein the “parent antibody” is the basis for modification to generate a variant of the present invention also referred to herein as a “bovinized antibody”. The term parent antibody may refer to the parent antibody itself, a composition comprising the parent antibody or the amino acid sequence or nucleic acid sequence that encodes the parent antibody.

The term “fully bovine recombinant monoclonal antibody” is intended to include recombinant antibodies having variable and constant regions derived from bovine germline immunoglobulin sequences. The fully bovine antibodies of the invention may include amino acid residues not encoded by the bovine germline immunoglobulin sequences (e.g., mutations introduced by random or site-specific mutagenesis in vitro or by somatic mutation in vivo). However, the term “fully bovine antibody”, as used herein, is not intended to include antibodies in which CDR sequences derived from the germline of another mammalian species, such as a mouse have been grafted onto bovine framework sequences.

In some embodiments the antibody is a full-length antibody. In some embodiments the full-length antibody comprises a heavy chain and a light chain.

A “therapeutic recombinant antibody” as that term is used herein is a recombinant antibody that use useful in preventing or ameliorating a disease, disorder or conditions when administered to a subject in need of treatment.

The term “recombinant antibody”, as used herein, is intended to include antibodies that are prepared, expressed, created or isolated by recombinant means including, but not limited to, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

“N-linked” oligosaccharides are those oligosaccharides that are linked to a peptide backbone through asparagine, by way of an asparagine-N-acetylglucosamine linkage. N-linked oligosaccharides are also called “N-linked glycans.” All N-linked oligosaccharides have a common pentasaccharide core of Man₃GlcNAc₂. They differ in the presence of, and in the number of branches (also called antennae) of peripheral sugars such as N-acetylglucosamine, galactose, N-acetylgalactosamine, fucose and sialic acid. Optionally, this structure may also contain a core fucose molecule and/or a xylose molecule.

“O-linked” oligosaccharides are those oligosaccharides also referred to herein as “0-linked glycans” are linked to a peptide backbone through threonine, serine, hydroxyproline, tyrosine, or other hydroxy-containing amino acids.

The term “sialic acid” (SA) refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al., JBC 1986; 261:11550-7; Kanamori et al., JBC 1990; 65:21811-9). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 1992; 2:25-40; Sialic Acids Chemistry, Metabolism and Function, R. Schauer, Ed. (Springer-Verlag, New York (1992)).

The term “sialylated” or “sialylation” refers to the addition of sialyl acid groups to oligosaccharide groups present on a glycosylated peptide such as an antibody. Such addition may be by natural enzymatic processes taking place in, for example a cell or via chemical glycoengineering.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid nucleic acid encoding additional peptide sequence.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides. The term “oligonucleotide” typically refers to short polynucleotides, generally no greater than about 50 nucleotides. Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a nucleic acid sequence encodes a protein if transcription and translation of mRNA corresponding to that nucleic acid produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that nucleic acid or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous” as used herein, refers to the subunit sequence similarity between two polymeric molecules, e.g., between two nucleic acid molecules, e.g., two DNA molecules or two RNA molecules, or between two peptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCCS' and 3′TATGGC share 50% homology.

As used herein, “homology” is used synonymously with “identity.” The determination of percent identity between two nucleotide or amino acid sequences can be accomplished using a mathematical algorithm. For example, a mathematical algorithm useful for comparing two sequences is the algorithm of Karlin and Altschul (PNAS 1990; 87:2264-8), modified as in Karlin and Altschul (PNAS 199390:5873-7). This algorithm is incorporated into the NBLAST and XBLAST programs of Altschul, et al. (JMB 1990; 215:403-10), and can be accessed, for example at the National Center for Biotechnology Information (NCBI) world wide web site having the universal resource locator “www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (Nucleic Acids Res 1997; 25:3389-402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See www.ncbi.nlm.nih.gov.

The percent identity between two sequences can be determined using techniques similar to those described above, with or without allowing gaps. In calculating percent identity, typically exact matches are counted.

A “heterologous nucleic acid expression unit” encoding a peptide is defined as a nucleic acid having a coding sequence for a peptide of interest operably linked to one or more expression control sequences such as promoters and/or repressor sequences wherein at least one of the sequences is heterologous, i.e., not normally found in the host cell.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a nucleic acid is able to promote transcription of the coding region.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

A “genetically engineered” or “recombinant” cell is a cell having one or more modifications to the genetic material of the cell. Such modifications are seen to include, but are not limited to, insertions of genetic material, deletions of genetic material and insertion of genetic material that is extrachromasomal whether such material is stably maintained or not.

As used herein, “native form” means the form of the peptide when produced by the cells and/or organisms in which it is found in nature. When the peptide is produced by a plurality of cells and/or organisms, the peptide may have a variety of native forms.

“Peptide” refers to a polymer in which the monomers are amino acids and are joined together through amide bonds, alternatively referred to as a peptide. Additionally, unnatural amino acids, for example, β-alanine, phenylglycine and homoarginine are also included. Amino acids that are not nucleic acid-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer thereof. The L-isomer is generally preferred. In addition, other peptidomimetics are also useful in the present invention. “Peptides” include, for example, oligopeptides, polypeptides, peptides, proteins, or glycoproteins.

As used herein, “polypeptide” refers to a chain comprising at least two consecutively linked amino acid residues, with no upper limit on the length of the chain. One or more amino acid residues in the protein may contain a modification such as, but not limited to, glycosylation, phosphorylation or a disulfide bond. A “protein” may comprise one or more polypeptides.

The present invention also provides for analogs of proteins or peptides which comprise a protein as identified above. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro, chemical derivatization of peptides, e.g., acetylation, or carboxylation. Also included are modifications of glycosylation, e.g., those made by modifying the glycosylation patterns of a peptide during its synthesis and processing or in further processing steps; e.g., by exposing the peptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

It will be appreciated, of course, that the peptides may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

As used herein, the term “MALDI” is an abbreviation for Matrix Assisted Laser Desorption Ionization. During ionization, SA-PEG (sialic acid-poly(ethylene glycol)) can be partially eliminated from the N-glycan structure of the glycoprotein.

As used herein, a “ruminant” is any mammal of the suborder Ruminantia which include domestic animals such as camel, deer, cows (bovine), sheep (ovine) and goats (caprine).

As used herein, “early colostrum” or “early colostral” includes colostrum produced 1 day to 7 days after the ruminant gives birth. In a preferred embodiment early colostrum is the colostrum produced within 1 to 3 days after birth. In a most preferred embodiment, early colostrum is the colostrum produced within 24 to 36 hours after birth.

As used herein, the “digestive tract” consists of the mouth, pharynx, esophagus, stomach, small intestine (duodenum, jejunum, ileum), large intestine (cecum, colon, rectum) and anus. In one preferred embodiment, the digestive tract is a human digestive tract.

As used herein, the “oral cavity” includes the mouth, the pharynx and the esophagus. The term “oral degradation” of an antibody is used herein to mean degradation of an antibody in the oral cavity by endogenous or exogenous enzymes present in the oral cavity.

As used herein, the “gastrointestinal tract”, or “GI tract” includes the stomach, small intestine (duodenum, jejunum, ileum), large intestine (cecum, colon, rectum) and anus.

As used herein, “intestinal digestion” refers to digestion in the small intestine and/or the large intestine.

As used herein, “intestinal degradation” of an antibody refers to degradation of an antibody in the small intestine and/or large intestine by endogenous or exogenous enzymes present in the small intestine and large intestine or due to exposure to acidic conditions during intestinal digestion.

As used herein, “enhanced” or “increased” ability to resist cleavage by proteases and/or enhanced or increased resistance to digestion or degradation during intestinal digestion in a mammal is intended to refer to a recombinant antibody or composition thereof that exhibits greater resistance to protease cleavage or intestinal digestion produced by the methods of the invention that impart the unique features of ruminant early colostral IgG antibodies that enable such antibodies to resist cleavage and digestion as compared to a recombinant antibody or composition thereof produced by a method that does not impart such features to the antibodies.

Proteases to which bovinized antibody variants of the invention have increased resistance may include, but are not limited to, simulated intestinal fluid, papain, pepsin, a matrix metalloproteinase including MMP-7, neutrophil elastase (HNE), stromelysin (MMP-3), macrophage elastase (MMP-12), trypsin, chymotrypsin, and other proteases as compared to antibodies that do not possess the features of the invention.

The term “antibody preparation” as used herein is used to define a composition comprising antibodies of the invention wherein contaminant components, such as materials which would interfere with diagnostic or therapeutic uses for the antibodies are substantially reduced. In an optional embodiment the antibody preparations of the invention will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain.

As used herein, “treatment” or “treating,” or “palliating” or “ameliorating” is used interchangeably herein. These terms refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect”, as used herein, refers to a physiologic effect, including but not limited to the cure, mitigation, amelioration, or prevention of disease in humans or other animals, or to otherwise enhance physical or mental well being of humans or animals, wherein the such therapeutic effect is facilitated by a recombinant antibody of the invention. Determination of a therapeutically effective amount is well within the capability of those skilled in the art, especially in light of the detailed disclosure provided herein.

The terms “therapeutically effective amount” and “therapeutically effective dose”, as used herein, refers to an amount of a recombinant antibody of the invention, either alone, or in combination with another therapeutic, that is capable of having any detectable, beneficial effect on any symptom, aspect, measured parameter or characteristics of a disease state or condition when administered in one or repeated doses to a subject. Such effect need not be absolute to be beneficial.

The term “therapeutically effective dose regimen”, as used herein, refers to a schedule for consecutively administered doses of a recombinant antibody of the invention, either alone or as part of a combination with another therapeutic, wherein the doses are given in therapeutically effective amounts to result in sustained beneficial effect on any symptom, aspect, measured parameter or characteristics of a disease state or condition.

“Altering the features of an antibody or composition of antibodies” to impart the features of early colostral IgG in accordance with the methods described herein is intended to encompass instances where the alteration of the features of an individual antibody, a subset of the antibodies in the compositions or all of the antibodies in the composition have been made to impart the unique features of a ruminant early colostral IgG antibody (e.g., a bovine colostral IgG1 antibody). Antibody resistance to proteolysis may be measured by any of the standard proteolysis assays known in the art.

The terms “patient” and “subject” refer to any human or non-human animal that receives either prophylactic or therapeutic treatment. For example, the methods and compositions described herein can be used to treat a subject having a gastrointestinal disorder. The term “non-human animal” includes all vertebrates, e.g., mammals and non-mammals, such as non-human primates, sheep, dog, cow, chickens, amphibians, reptiles, etc.

As used herein, “cattle” refer to members of the subfamily Bovinae, and include the species Bos indicus and Bos taurus. As used herein, “cattle” is intended to encompass calves, mature cows, steers, and bulls.

Various aspects described herein are described in further detail in the following subsections.

### Overview

Described herein are recombinant antibodies having an increased ability to resist cleavage by proteases and resist degradation during digestion in the digestive tract of a mammal (e.g., during human digestion), as well as methods for preparing the same.

Bovine antibodies, and particularly early colostral bovine IgG, are known to have a natural resistance to digestion in the human gastrointestinal tract (Warny et al., Gut 1999; 44:212-217). The basis of this resistance was previously unknown. The present invention is based, at least in part, on the discovery of the macromolecular basis that imparts to bovine IgG1 enhanced resistance to proteases and other digestive enzymes present in the gut in vitro and in vivo.

The initial secretion present in the mammary gland at or near the time of parturition is termed colostrum, which arises during a distinct physiological and functional stage of mammary gland development that is completely different from the gland's primary role of milk production. During colostrogenesis, the transfer of immunoglobulins from the maternal circulation into mammary secretions in domestic ruminants begins several weeks prior to parturition and ceases after parturition. During this time 500 g-3 kg of IgG are transferred into mammary secretions and early studies have established that the source of colostral immunoglobulin is the maternal circulation.

Colostrum is unique in its composition and function. In domestic ruminants the principal difference between colostrum and milk is the high concentration of colostral immunoglobulin, specifically IgG1. IgG1 is concentrated to levels 5-10 times that of serum where it accounts for greater than 90% of total colostral protein. The process by which immunoglobulin is transferred from maternal circulation into the mammary gland is the result of transcytosis, a process by which various macromolecules including immunoglobulins are transported across the interior bovine mammary epithelial cells. Immunoglobulins are captured in vesicles on the basal-lateral side of the mammary epithelium cell, drawn across the cell and ejected at the apical surface into colostrum.

As will be discussed in further detail below, described herein, for the first time, are several structural differences between the IgG found in bovine serum and colostrum, and in particular, between IgG isotypes, that impart protease resistance to bovine colostral antibodies. The antibodies described herein take advantage of these structural differences to generate protease-resistant antibodies.

### Bovinized Antibodies

Provided herein are recombinant antibodies having unique structural features associated with bovine IgG1 antibodies that impart enhanced protease resistance and which can be orally administered for the treatment diseases of the digestive tract.

Accordingly, provided herein are recombinant antibodies that retain at least the antigen binding CDRs or variable region (VR) from a parent (non-bovine) antibody, and includes all or a portion of a bovine IgG1 constant region (e.g., a bovine IgG1 CH1 and/or hinge domain). Such antibodies are referred to herein as “bovinized antibodies.” Also provided herein are bovinized antibodies which comprises the variable region of a parent antibody, and the constant region (i.e., CH1 domain, hinge region, CH2 domain, and CH3 domain) of a bovine IgG1 antibody. These antibodies are referred to as “bovine chimeric antibodies.”

In one aspect, provided herein are bovinized antibodies that fully or partially replace one or more constant region domain(s) of a parent (i.e., non-bovine) antibody with the equivalent bovine IgG1 constant region domain(s).

Exemplary bovine IgG1 antibody constant regions are presented in Table 1.

TABLE 1

| | Accession | |
|---|---|---|
| Allotype | ID | Sequence |
| IgG1a | S82409 | ASTTAPKVYPLSSCCGDKSSSTVTLGCLVSSYMPEPVTVTWNSGALKSGVHTFPAVLQSSGL |
| (SEQ ID | | YSLSSMVTVPGSTSGTQTFTCNVAHPASSTKVDKAVDPRCKTTCDCCPPPELPGGPSVFIFP |
| NO: 1) | | PKPKDTLTISGTPEVTCVVVDVGHDDPEVKFSWFVDDVEVNTATTKPREEQFNSTYRVVSAL |
| | | RIQHQDWTGGKEFKCKVHNEGLPAPIVRTISRTKGPAREPQVYVLAPPQEELSKSTVSLTCM |
| | | VTSFYPDYIAVEWQRNGQPESEDKYGTTPPQLDADGSYFLYSRLRVDRNSWQEGDTYTCVVM |
| | | HEALHNHYTQKSTSKSAGK |
| IgG1b | X16701 | ASTTAPKVYPLSSCCGDKSSSTVTLGCLVSSYMPEPVTVTWNSGALKSGVHTFPAVLQSSGL |
| (SEQ ID | | YSLSSMVTVPGSTSGQTFTCNVAHPASSTKVDKAVDPTCKPSPCDCCPPPELPGGPSVFIFP |
| NO: 2) | | PKPKDTLTISGTPEVTCVVVDVGHDDPEVKFSWFVDDVEVNTATTKPREEQFNSTYRVVSAL |
| | | RIQHQDWTGGKEFKCKVHNEGLPAPIVRTISRTKGPAREPQVYVLAPPQEELSKSTVSLTCM |
| | | VTSFYPDYIAVEWQRNGQPESEDKYGTTPPQLDADSSYFLYSKLRVDRNSWQEGDTYTCVVM |
| | | HEALHNHYTQKSTSKSAGK |
| IgG1c | DQ452014 | ASTTAPKVYPLSSCCGDKSSSTVTLGCLVSSYMPEPVTVTWNSGALKSGVHTFPAVLQSSGL |
| (SEQ ID | | YSLSSMVTVPASSSGTQTFTCNVAHPASSTKVDKAVDPRCKRPCDCCPPPELPGGPSVFIFP |
| NO: 3) | | PKPKDTLTISGTPEVTCVVVDVGHDDPEVKFSWFVDNVEVNTATTKPREEQFNSTYRVVSAL |
| | | RIQHQDWTGGKEFKCKVHNEGLPAPIVRTISRTKGPAREPQVYVLAPPQEELSKSTVSLTCM |
| | | VTSFYPDYIAVEWQRNGQPESEDKYGTTPPQLDADSSYFLYSKLRVDRNSWQEGDTYTCVVM |
| | | HEALHNHYTQKSTSKSAGK |
| IgG1d | X62916 | ASTTAPKVYPLSSCCGDKSSSTVTLGCLVSSYMPEPVTVTWNSGALKSGVHTFPAVLQSSGL |
| (SEQ ID | | YSLSSMVTVPGSTSGQTFTCNVAHPASSTKVDKAVDPTCKPSPCDCCPPPELPGGPSVFIFP |
| NO: 2) | | PKPKDTLTISGTPEVTCVVVDVGHDDPEVKFSWFVDDVEVNTATTKPREEQFNSTYRVVSAL |
| | | RIQHQDWTGGKEFKCKVHNEGLPAPIVRTISRTKGPAREPQVYVLAPPQEELSKSTVSLTCM |
| | | VTSFYPDYIAVEWQRNGQPESEDKYGTTPPQLDADSSYFLYSKLRVDRNSWQEGDTYTCVVM |
| | | HEALHNHYTQKSTSKSAGK |

In one embodiment, provided are bovinized antibodies that incorporate 1, 2, 3, or all of the following structural features found in bovine IgG1 that impart protease resistance to the antibody:
1) a disulfide bond linking the N-terminus of the CH1 domain to the N-terminus of the hinge domain (“bovine CH1-hinge disulfide linkage”),
2) a disulfide bond linking the N-terminus of the CH1 domain to the C-terminus of the light chain (“bovine CH1-light chain disulfide linkage”),
3) a cluster of three disulfide bonds linking the hinge regions of two heavy chains together (bovine heavy chain-heavy chain disulfide linkage”), and/or
4) a hinge sequence that is less susceptible to common proteases (“bovinized hinge”).

In some embodiments, the bovinized antibody comprises the variable domain, CH2 domain, and CH3 domain of the parent antibody, and at least a portion (i.e., one or more amino acid residues) of a bovine IgG1 CH1 and/or hinge domain. In some embodiments, the CH1 domain of the antibody is at least 85% identical, for example, at least 90%, at least 95%, at least 98%, at least 99%, or at least 100% identical, to the bovine IgG1 CH1 and/or hinge domain.

In particular embodiments, the bovinized antibodies described herein comprise a CH1 domain from a parent antibody (e.g., a human CH1 domain), wherein the amino acid residues equivalent to Cys 127 and Cys 128 (according to Kabat numbering) in bovine IgG1 are replaced with cysteine in the parent antibody. For example, in one embodiment, the antibody comprises a human CH1 domain with S127C and/or S128C substitutions (numbering according to Kabat). In another embodiment, the antibody comprises a human CH1 domain with S127C and/or S128C amino acid substitutions, wherein 1, 2, 3, 4, 5, 6, 7, 8, 9, or 10 amino acids upstream (N-terminal) and/or 1, 2, 3, 4, 5, 6, 7, 8, 9, or 10 amino acids downstream (C-terminal) of amino acid residues 127 and 128 are replaced with the equivalent bovine IgG1 CH1 residues.

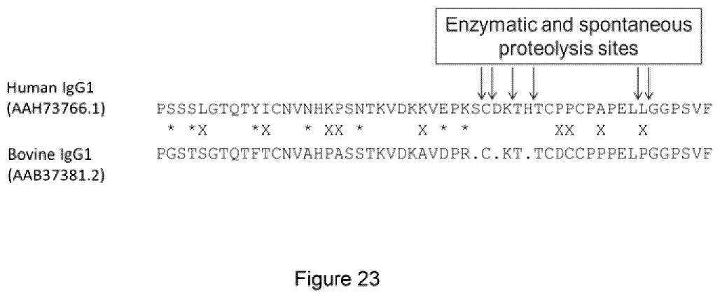

In other embodiments, the bovinized antibody comprises a bovinized hinge region. For example, in one embodiment, the antibody comprises a human IgG hinge, wherein sites within the hinge which are susceptible to enzymatic or spontaneous proteolysis are replaced with the equivalent bovine hinge region sequences. As shown in FIG. 23, the bovine IgG1 hinge region sequence differs from the human IgG1 hinge region sequence at sites known to be susceptible to protease digestion.

In further embodiments, the bovinized antibody comprises a bovinized hinge region, in which some or all of the amino acid residues of the hinge region are replaced with the equivalent portion of the bovine IgG1 hinge region sequence.

In still further embodiments, the bovinized antibody comprises a bovinized hinge region which comprises a bovine heavy chain-heavy chain linkage, wherein a cluster of three disulfide bonds link the hinge regions of two heavy chains together. Accordingly, in one embodiment, the antibody comprises a hinge of the parent antibody, wherein the amino acid residues equivalent to the underlined cysteine residues in the bovine hinge region below are replaced with cysteine (if not already cysteine) in the hinge of the parent antibody.

(SEQ ID NO: 4)
DKAVDPRCKPSPCDCCPPPELPGGP

In one embodiment, the antibody comprises a human hinge region sequence, wherein Pro241 (Kabat numbering) is replaced with a cysteine residue (i.e., a P241C substitution).

In some embodiments, the bovinized antibodies exhibit greater protease resistance relative to the parent antibody.

In some embodiments, the bovinized antibodies retain greater antigen-binding activity after protease digestion relative to the parent antibody.

In some embodiments, the bovinized antibodies, upon being subjected to protease digestion in the GI tract, retain antigen-binding activity. In certain embodiments, the bovinized antibodies remain bivalent upon digestion by proteases in the GI tract. In one embodiment, the bovinized antibodies are digested into F(ab′)₂ fragments by proteases in the GI tract.

In some embodiments, the protease resistance of bovinized antibodies comprising a bovine IgG1 hinge is increased by partially incorporating bovine IgG3 hinge region sequences into the IgG1 hinge region. Without being bound by theory, pepsin cleavage can be prevented by adding a bovine IgG3 hinge domain having a putative O-glycosylation site to the bovine IgG1 constant region.

In certain embodiments, the antigen-binding region of the bovinized antibodies lack protease-sensitive sequences. Such determinations can be made by comparing the primary sequence of the variable region (e.g., the CDR regions) with known protease consensus sequences, or tested experimentally using pancreatin digestion methods described in the Examples.

In some embodiments, portions of the constant regions of other ruminants that share structural similarities with the bovine constant region that imparts protease stability can be incorporated into the recombinant antibodies described herein. For instance, Example 9 provides the CH1-hinge region sequences of other ruminant species which share structural similarities with the corresponding bovine sequences. In certain embodiments, these structurally similar sequences are derived from the rabbit IgG1 constant region.

In some embodiments, the bovinized antibody comprises one or more additional amino acid substitutions (e.g., 1, 2, 3, 4, or 5 amino acid substitutions) in the hinge or Fc region that increase or decrease antibody effector activity and/or FcRn binding activity.

Particular amino acid substitutions include those that generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fc.

Although the passages below generally relate to substitutions and positions in the human Fc region, the skilled artisan could readily introduce corresponding substitutions at equivalent positions of the Fc regions of other species.

In some embodiments, the Fc region is altered by replacing at least one amino acid residue with a different amino acid residue to alter the effector function(s) of the antibody.

For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. See, e.g., U.S. Pat. Nos. 5,624,821 and 5,648,260.

In another example, one or more amino acids selected from amino acid residues 329, 331 and 322 can be replaced with a different amino acid residue such that the antibody has altered C1q binding and/or reduced or abolished complement dependent cytotoxicity (CDC). See, e.g., U.S. Pat. No. 6,194,551.

In another example, one or more amino acid residues within amino acid positions 231 and 239 are altered to thereby alter the ability of the antibody to fix complement. See, e.g., PCT Publication WO 94/29351.

In yet another example, the Fc region may be modified to increase antibody dependent cellular cytotoxicity (ADCC) and/or to increase the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 3241, 332D, and 332E. Exemplary variants include 239D/332E, 236A/332E, 236A/239D/332E, 268F/324T, 267E/268F, 267E/3241, and 267E/268F/324T. Other modifications for enhancing FcyR and complement interactions include but are not limited to substitutions 298A, 333A, 334A, 326A, 2471, 339D, 339Q, 280H, 290S, 298D, 298V, 243L, 292P, 300L, 396L, 305I, and 396L These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Kabat (WO00/42072).

Other Fc modifications that can be made to Fcs are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcyR and complement interactions include substitutions 297A, 234A, 235A, 237A, 318A, 228P, 236E, 268Q, 309L, 330S, 331 S, 2205, 2265, 2295, 238S, 233P, and 234V, as well as removal of the glycosylation at position 297 by mutational or enzymatic means or by production in organisms such as bacteria that do not glycosylate proteins, See, e.g., Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCT Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

Fc variants that enhance affinity for an inhibitory receptor FcyRIIb may also be used. Such variants may provide an Fc fusion protein with immunomodulatory activities related to FcyRIIb⁺ cells, including for example B cells and monocytes. In one embodiment, the Fc variants provide selectively enhanced affinity to FcyRIIb relative to one or more activating receptors. Modifications for altering binding to FcyRIIb include one or more modifications at a position selected from the group consisting of 234, 235, 236, 237, 239, 266, 267, 268, 325, 326, 327, 328, and 332, according to the EU index. Exemplary substitutions for enhancing FcyRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcyRIIb include 235Y/267E, 236D/267E, 239D/268D 239D/267E, 267E/268D, 267E/268E, and 267E/328F.

In some embodiments, the hinge is a bovinized hinge, and comprises one or more of the amino acid substitutions described in Example 10.

In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants include: S298A/E333A/L334A, S239D/I332E, S239D/1332E/A330L, L235V/F243L/R292P/Y300L/P396L, and M428L/N434S.

Fc variants that enhance affinity for Protein A from Staphylococcus areus may also be used. Previous work (US Patent Application US20140154270A1) has shown that IgG from ruminant species binds to Protein A with poor affinity under conditions typically used for manufacturing. Variants of the bovine sequence may provide an Fc fusion protein that can be purified by methods commonly used in the manufacture of biological therapeutics. In some embodiments, the antibody can be modified at positions 250-255, 288, 307-317, or 430-436 to amino-acids found in IgG molecules known to bind Protein A with high affinity. In a subset of these embodiments, substitutions may be chosen to enhance Protein A binding while retaining weak binding to human FcRn. Without being bound by mechanism, these substitutions prevent the antibody from being transported from the gut into systemic circulation. Accordingly, in certain embodiments, the Fc domain contains one or more (i.e., 1, 2, 3, 4, or 5) of the following substitutions: T252M, G255R, 0309L, T314L, and G315N. In one embodiment, the Fc domain contains a T252M/G255R substitution. In another embodiment, the Fc domain contains a Q309L/T314L/G315N substitution. In yet another embodiment, the Fc domains contains a T252M/G255R/Q309L/T314L/G315N substitution.

In one embodiment, the parent antibody comprises a hinge region, or is altered to comprise a hinge region, that is at least 85%, such as at least 90%, at least 95%, at least 98%, at least 99%, or 100% identical to the equivalent portion of a hinge region comprising the amino acid sequence of bovine IgG1 (S22080): DKAVDPRCKPSPCDCCPPPELPGGP (SEQ ID NO: 4). In another embodiment, the parent antibody comprises a hinge region, or is altered to comprise a hinge region, that is at least 85%, such as at least 90%, at least 95%, at least 98%, at least 99%, or 100% identical to the equivalent portion of a hinge region comprising the amino acid sequence of selected from the following hinge region amino acid sequences of Table 2:

TABLE 2

| SEQ | | |
|---|---|---|
| ID | Species | Sequence |
| 5 | Bos Taurus (cow) | VDK--AVDP---RCK-TTCD-C-CPPPELPGGPSVF |
| 6 | Bos Taurus (cow) | VDK--AVDP---RCK-RPCD-C-CPPPELPGGPSVF |
| 7 | Bos Taurus (cow) | VDK--AVDP---TCKPSPCD-C-CPPPELPGGPSVF |
| 8 | Ovis aries (sheep) | VDK--RVEP---GCP-DPCKHCRCPPPELPGGPSVF |
| 8 | Ovis aries (sheep) | VDK--RVEP---GCP-DPCKHCRCPPPELPGGPSVF |
| 8 | Ovis aries (sheep) | VDK--RVEP---GCP-DPCKHCRCPPPELPGGPSVE |
| 9 | Lama glama (llama) | VDK--RVEPHG-GCT---CP--QCPAPELPGGPSVF |
| 9 | Vicugna pacos (alpaca) | VDK--RVEPHG-GCT---CP--QCPAPELPGGPSVF |
| 9 | Camelus dromedarius (Arabian | VDK--RVEPHG-GCT---CP--QCPAPELPGGPSVF |
| | camel) | |
| 10 | Meriones unguiculatus (gerbil) | VDK--TVEPRGTKHICPDCP--KCPAPDLSGGPSVF |
| 11 | Felis catus (cat) | VDKTVRKTDHP-PGP-KPCDCPKCPPPEMLGGPSIF |

In one embodiment, the recombinant antibody comprises all or a portion of a hinge region that is at least 85%, such as at least 90%, at least 95%, at least 98%, at least 99%, or 100% identical to the amino acid sequence of the equivalent portion of a ruminant antibody hinge region (e.g., a bovine IgG1 hinge region) and optionally comprises all or a portion of a CH1 domain that is at least 85%, at least 90%, at least 95%, at least 98%, at least 99%, at least 100% identical to the amino acid sequence of the equivalent portion of a ruminant antibody (e.g., a bovine IgG1 hinge region) and further optionally comprises all or a portion of a CH1 domain that is at least 85%, at least 90%, at least 95%, at least 98%, at least 99%, at least 100% identical to the amino acid sequence of the equivalent portion of a ruminant antibody (e.g., a bovine IgG1 hinge region). Exemplary bovine hinge regions are presented in Table 3.

TABLE 3

| | Accession | |
|---|---|---|
| Allotype | ID | Sequence |
| IgG1a | S82409 | DPRCKTTCDCCP |
| (SEQ ID | | |
| NO: 12) | | |
| IgG1b | X16701 | DPTCKPSPCDCCP |
| (SEQ ID | | |
| NO: 13) | | |
| IgG1c | DQ452014 | DPRCKRPCDCCP |
| (SEQ ID | | |
| NO: 14) | | |
| IgG1d | X62916 | DPTCKPSPCDCCP |
| (SEQ ID | | |
| NO: 13) | | |

In one embodiment, the parent antibody comprises or is altered to comprise a hinge region and a CH1 domain is at least 85%, such as at least 90%, at least 95%, at least 98%, at least 99%, or 100% identical to the equivalent portion of a bovine, CH1 domain amino acid sequence (e.g., a bovine IgG1 CH1 domain). Exemplary bovine IgG1 CH1 domains are presented in Table 4.

TABLE 4

| | Accession | |
|---|---|---|
| Allotype | ID | Sequence |
| IgG1a | S82409 | ASTTAPKVYPLSSCCGDKSSSTVTLGCLVSSYMPEPVTVTWNSGALKSGVHTFP |
| (SEQ ID | | AVLQSSGLYSLSSMVTVPGSTSGTQTFTCNVAHPASSTKVDKAV |
| NO: 15) | | |
| IgG1b | X16701 | ASTTAPKVYPLSSCCGDKSSSTVTLGCLVSSYMPEPVTVTWNSGALKSGVHTFP |
| (SEQ ID | | AVLQSSGLYSLSSMVTVPGSTSGQTFTCNVAHPASSTKVDKAV |
| NO: 16) | | |
| IgG1c | DQ452014 | ASTTAPKVYPLSSCCGDKSSSTVTLGCLVSSYMPEPVTVTWNSGALKSGVHTFP |
| (SEQ ID | | AVLQSSGLYSLSSMVTVPASSSGTQTFTCNVAHPASSTKVDKAV |
| NO: 17) | | |
| IgG1d | X62916 | ASTTAPKVYPLSSCCGDKSSSTVTLGCLVSSYMPEPVTVTWNSGALKSGVHTFP |
| (SEQ ID | | AVLQSSGLYSLSSMVTVPGSTSGQTFTCNVAHPASSTKVDKAV |
| NO: 16) | | |

In one embodiment, the parent antibody comprises or is altered to comprise a hinge region and a CH2 domain is at least 85%, such as at least 90%, at least 95%, at least 98%, at least 99%, or 100% identical to the equivalent portion of a bovine, CH2 domain amino acid sequence (e.g., a bovine IgG1 CH2 domain). Exemplary bovine IgG1 CH2 domains are presented in Table 5.

TABLE 5

| | Accession | |
|---|---|---|
| Allotype | ID | Sequence |
| IgG1a | S82409 | PPELPGGPSVFIFPPKPKDTLTISGTPEVTCVVVDVGHDDPEVKFSWFVDDVEV |
| (SEQ ID | | NTATTKPREEQFNSTYRVVSALRIQHQDWTGGKEFKCKVHNEGLPAPIVRTISR |
| NO: 18) | | TK |
| IgG1b | X16701 | PPELPGGPSVFIFPPKPKDTLTISGTPEVTCVVVDVGHDDPEVKFSWFVDDVEV |
| (SEQ ID | | NTATTKPREEQFNSTYRVVSALRIQHQDWTGGKEFKCKVHNEGLPAPIVRTISR |
| NO: 18) | | TK |
| IgG1c | DQ452014 | PPELPGGPSVFIFPPKPKDILTISGTPEVTCVVVDVGHDDPEVKFSWFVDNVEV |
| (SEQ ID | | NTATTKPREEQFNSTYRVVSALRIQHQDWTGGKEFKCKVHNEGLPAPIVRTISR |
| NO: 19) | | TK |
| IgG1d | X62916 | PPELPGGPSVFIFPPKPKDTLTISGTPEVTCVVVDVGHDDPEVKFSWFVDDVEV |
| (SEQ ID | | NTATTKPREEQFNSTYRVVSALRIQHQDWTGGKEFKCKVHNEGLPAPIVRTISR |
| NO: 18) | | TK |

In one embodiment, the parent antibody comprises or is altered to comprise a hinge region and a CH3 domain is at least 85%, such as at least 90%, at least 95%, at least 98%, at least 99%, or 100% identical to the equivalent portion of a bovine, CH3 domain amino acid sequence (e.g., a bovine IgG1 CH3 domain). Exemplary bovine IgG1 CH3 domains are presented in Table 6.

TABLE 6

| | Accession | |
|---|---|---|
| Allotype | ID | Sequence |
| IgG1a | S82409 | GPAREPQVYVLAPPQEELSKSTVSLTCMVTSFYPDYIAVEWQRNGQPESEDKYG |
| (SEQ ID | | TTPPQLDADGSYFLYSRLRVDRNSWQEGDTYTCVVMHEALHNHYTQKSTSKSAG |
| NO: 20) | | K |
| IgG1b | X16701 | GPAREPQVYVLAPPQEELSKSTVSLTCMVTSFYPDYIAVEWQRNGQPESEDKYG |
| (SEQ ID | | TTPPQLDADSSYFLYSKLRVDRNSWQEGDTYTCVVMHEALHNHYTQKSTSKSAG |
| NO: 21) | | K |
| IgG1c | DQ452014 | GPAREPQVYVLAPPQEELSKSTVSLTCMVTSFYPDYIAVEWQRNGQPESEDKYG |
| (SEQ ID | | TTPPQLDADSSYFLYSKLRVDRNSWQEGDTYTCVVMHEALHNHYTQKSTSKSAG |
| NO: 21) | | K |
| IgG1d | X62916 | GPAREPQVYVLAPPQEELSKSTVSLTCMVTSFYPDYIAVEWQRNGQPESEDKYG |
| (SEQ ID | | TTPPQLDADSSYFLYSKLRVDRNSWQEGDTYTCVVMHEALHNHYTQKSTSKSAG |
| NO: 21) | | K |

In some embodiments, the parent antibody comprises or is altered to comprise a fusion between a VL domain with specific binding to a desired antigen and a bovine CL domain. In a preferred embodiment, the parent antibody contains a CL domain corresponding to the encoded protein from the bovine IGLC1, IGLC2, IGLC3 or IGLC4 loci.

In one embodiment, the recombinant antibodies are capable of surviving digestion and reaching their target antigens in the digestive tract. In another embodiment, the recombinant antibodies are therapeutic antibodies that are bovinized as described herein or variants of existing therapeutic antibodies modified to increase resistance to cleavage by proteases and resist degradation during digestion in the digestive tract of a mammal, such as during human digestion.

In one embodiment, the recombinant parent antibody is specific for a target antigen. In some embodiments, the target antigen is a biological antigen (e.g., a human protein, a human peptide, or other antigenic molecule specific to humans), such as the antigens described further herein. In other embodiments, the target antigen is not a synthetic antigen, such as a dye. In one embodiment, the antibody is not an anti-dansyl antibody. In one embodiment, the recombinant parent antibody is targeted to an antigen not normally present in a ruminant in the absence of immunization of the ruminant with the target antigen. In one embodiment, the recombinant parent antibody is specific for a target antigen that is a protein, peptide, or other antigenic molecule derived from another mammal.

In certain embodiments, the target antigen is a therapeutic target antigen and the bovinized antibody facilitates a therapeutic effect on a subject when administered to the subject.

Table 7 provides a partial list of various therapeutic fusion proteins that are suitable as parent antibodies for the production of the bovinized antibodies described herein.

TABLE 7

| Agent | Vendor | Cell Source |
|---|---|---|
| Monoclonal | | |
| Antibodies | | |
| Actemra ® | Genentech Inc. | CHO cells |
| Tocilizumab | | |
| Avastin ® | Genentech Inc., Hoffmann-La | CHO cells |
| Bevacizumab | Roche Ltd. | |
| Campath ® (US), | Genzyme Corp. | CHO cells |
| Mabcampath ® | | |
| (EU) | | |
| Alemtuzumab | | |
| Herceptin ® | F. Hoffmann-La Roche Ltd, | CHO cells |
| Trastuzumab | Genentech Inc. | |
| Humira ® | Abbott Laboratories | CHO cells |
| Adalimumab | | |
| Rituxan ® | Genentech | CHO cells |
| Rituximab | | |
| Simponi ® | Centocor Ortho Biotech Inc., | CHO cells |
| Golimumab | Johnson & Johnson Co., | |
| | Schering-Plough Corp. | |
| Stelara ™ | Centocor Ortho Biotech Inc. | CHO cells |
| Ustekinumab | | |
| Vectibix ® | Amgen | CHO cells |
| Panitumumab | | |
| Xolair ® | Genentech Inc., Novartis | CHO cells |
| Omalizumab | Pharmaceuticals Corp. | |
| | Tanox Inc. | |
| Zevalin ® | Biogen Idec., Schering AG | CHO cells |
| Ibritumomab tiuxetan | | |
| Bexxar ® | GlaxoSmithKline | Hybridoma, |
| Tositumomab-I131 | | mammalian |
| Soliris ® | Alexion Pharmaceuticals, Inc | Murine |
| Eculizumab | | myeloma |
| | | cell line |
| Ilaris ® | Novartis Pharmaceuticals Corp. | Murine |
| Canakinumab | | Sp2/0-Ag14 |
| | | fused |
| | | hybridoma |
| | | cell line |
| Mylotarg ® | Wyeth Pharmaceuticals | NS0 mouse |
| Gemtuzumab | | myeloma cells |
| ozogamicin | | |
| Arzerra ® | GlaxoSmithKline | NSO mouse |
| Ofatumumab | | myeloma cells |
| Synagis ® | Abbott Laboratories, | NSO mouse |
| Palivizumab | MedImmune Inc. | myeloma cells |
| Tysabri ® | Élan Pharmaceuticals, | NSO mouse |
| Natalizumab | Biogen Idec. | myeloma cells |
| Erbitux ® | ImClone Systems | Sp2/0 mouse |
| Cetuximab | Merck & Co., Inc., Bristol- | myeloma cells |
| | Myers Squibb | |
| Remicade ® | Centocor Ortho Biotech Inc. | Sp2/0 mouse |
| Infliximab | | myeloma cells |
| Reopro ® | Centocor Ortho Biotech Inc., | Sp2/0 mouse |
| Abciximab | Eli Lilly & Co. | myeloma cells |
| Simulect ® | Novartis Pharmaceuticals Corp. | Sp2/0 mouse |
| Basiliximab | | myeloma cells |
| Zenapax ® | F. Hoffmann-La Roche Ltd., | Sp2/0 mouse |
| Daclizumab | PDL (Protein Design Labs) | myeloma cells |
| | BioPharma | |

Table 8 provides a non-limiting list of fusion proteins that are suitable parent antibodies for bovinization in accordance with the invention.

TABLE 8

| Amevive ® | Astellas Pharma Inc. | CHO cells |
|---|---|---|
| Alefacept | | |
| Arcalyst ® | Regeneron Pharmaceuticals Inc. | CHO cells |
| Rilonacept | | |
| Enbrel ® | Amgen, Wyeth Pharmaceutical | CHO cells |
| Etanercept | | |
| Orencia ® | Bristol-Myers-Squibb | CHO cells |
| Abatacept | | |
| Hormones | | |
| Follistim ® | Schering-Plough Corp. | CHO cells |
| Follitropin beta | | |
| Gonal-F ® | EMD Serono, Inc. | CHO cells |
| Follitropin alfa | | |
| Luveris ® | EMD Serono, Inc. | CHO cells |
| Luteinizing hormone | | |
| OP-1 Putty | Stryker Biotech | CHO cells |
| Osteogenic Protein-1 | | |
| (BMP-7) | | |
| Ovidrel ® | EMD Serono, Inc. | CHO cells |
| Choriogonadotropin α | | |
| Thyrogen ® | Genzyme Corp | CHO cells |
| Thyrotropin alfa | | |
| Serostim ®, Saizen ®, | EMD Serono, Inc. | Murine cell |
| Zorbtive ™ | | line (mouse |
| Somatropin | | C127) |
| Cytokines | | |
| Aranesp ® | Amgen | CHO cells |
| Darbepoetin alfa | | |
| Avonex ® | Biogen Idec, Inc. | CHO cells |
| Interferon beta-1a | | |
| Neorecormon ® | Hoffmann-La Roche Ltd. | CHO cells |
| Epoetin beta | | |
| Procrit ®, Epogen ® | Amgen, Centocor Ortho | CHO cells |
| Epoetin alfa | Biotech Inc. | |
| Rebif ® | Pfizer, Inc., EMD Serono, Inc. | CHO cells |
| Interferon beta-1a | | |
| Clotting Factors | | |
| Helixate FS | ZLB Behring | BHK cells |
| Coagulation | | |
| factor VIII | | |
| Kogenate FS | Genentech | BHK cells |
| Coagulation | | |
| factor VIII | | |
| NovoSeven ®, | Novo Nordisk | BHK cells |
| Coagulation | | |
| Factor VIIa | | |
| Advate ® | Baxter International Inc. | CHO cells |
| Antihemophilic factor | | |
| BeneFIX ® | Wyeth Pharmaceuticals | CHO cells |
| Coagulation | | |
| Factor IX | | |
| ReFacto ® | Wyeth Pharmaceuticals | CHO cells |
| Antihemophilic Factor | | |
| Xyntha ® | Wyeth Pharmaceuticals | CHO cells |
| Coagulation | | |
| factor VIII | | |
| Xigris ® | Eli Lilly & Co. | HEK293 |
| Drotrecogin alfa | | |
| (Activated Protein C) | | |
| Enzymes | | |
| Activase ®, Cathflo | Genentech, Boehringer | CHO cells |
| Activase ®, | Ingelheim Pharma KG | |
| Actilyse ® | | |
| Alteplase | | |
| Aldurazyme ® | Genzyme Corp | CHO cells |
| Laronidase | | |
| Cerezyme ® | Genzyme Corp. | CHO cells |
| Imiglucerase | | |
| Fabrazyme ® | Genzyme Corp | CHO cells |
| agalsidase-β | | |
| Hylenex ®, | MediCult A/S, MidAtlantic | CHO cells |
| Cumulase ® | Diagnostics, Inc., Halozyme | |
| Hyaluronidase | Baxter Healthcare | |
| Myozyme ® | Genzyme Corp | CHO cells |
| Alglucosidase alfa | | |
| Naglazyme ® | BioMarin Pharmaceutical Inc. | CHO cells |
| N- | | |
| acetylgalactosamine | | |
| 4-sulfatase | | |
| Pulmozyme ® | Genentech, Hoffmann-La | CHO cells |
| Human DNase | Roche Ltd. | |
| TNKase ® | Genentech | CHO cells |
| Tenecteplase | | |
| Elaprase ® | Shire Pharmaceuticals | human cell |
| Idursulfase | | line (HT- |
| | | 1080) |

Bovinized antibodies can be prepared using standard recombinant technologies. Antibody fragments for example, which contain specific binding sites of the target protein of interest, may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., Science 1989; 246:1275-81) to allow rapid and easy identification of monoclonal Fab fragments with the desired specificity to the target protein of interest.

Also provided herein are antibodies (bovinized antibodies and fully bovine recombinant monoclonal IgG1 antibodies) which bind to the same epitope on a target of interest as a reference antibody (e.g., a therapeutic antibody), or competes with a reference antibody for binding to a target of interest. Whether a particular antibody binds to the same epitope on a target antigen as a reference antibody, or competes with binding to the same epitope on a target antigen as a reference antibody, can be readily determined by the skilled artisan using art-recognized epitope mapping and competition assays, respectively.

The antibodies described herein (bovinized antibodies and fully bovine recombinant monoclonal IgG1 antibodies) may be used to generate bispecific molecules. For instance, the antibodies can be derivatized or linked to another functional molecule, e.g., another peptide or protein (e.g., another antibody or ligand for a receptor) to generate a bispecific molecule that binds to at least two different binding sites or target molecules using methods known in the art.

In another aspect, provided herein are immunoconjugates comprising the antibodies (bovinized antibodies and fully bovine recombinant monoclonal IgG1 antibodies) described herein conjugated to a therapeutic moiety, e.g., a cytotoxin, drug, or radioisotope. Exemplary cytotoxins include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestostexone, glucocorticoids, procaine, tetracaine, lidocaine, propranolol, and puromycin and analogs or homologs thereof.

Suitable therapeutic agents for forming immunoconjugates include, e.g., akylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), antibiotics (e.g., dactinomycin (formerly actinomycin), bleomycin, mithramycin, and anthramycin (AMC)), antimetabolites (e.g., methotrexate, 6-mercaptopurine, 6-thioguanine, cytarabine, fludarabin, 5-fluorouracil decarbazine), anthracyclines (e.g., daunorubicin (formerly daunomycin) and doxorubicin), and anti-mitotic agents (e.g., vincristine and vinblastine).

The antibodies (bovinized antibodies and fully bovine recombinant monoclonal IgG1 antibodies) described herein can be conjugated to a radioisotope, e.g., iodine-131, yttrium-90 or indium-111, to generate cytotoxic radiopharmaceuticals for treating cancers. The antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon-γ; or, biological response modifiers such as, for example, lymphokines, interleukin-1 (“IL,1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Methods for conjugating therapeutic moieties to antibodies are well known in the art.

In some embodiments, the antibodies described herein are administered systemically. In a preferred embodiment, the antibody is not immunogenic in the subject being treated. Such antibodies can be designed to avoid increasing the predicted risk for T cell epitopes, for example, as assessed using art-recognized algorithms and databases (see, e.g., Vita et al., Nucleic Acids Res 2015; 43:D405-12; Vita et al., Nucleic Acids Res 38:D854-62, Nielsen et al., Protein Sci 2003; 12:1007-17; Bui et al., Immunogenetics 2005; 57:304-14; Lundegaard et al., J Immunol Methods 2011; 374:226-34). In certain embodiments, mutations are made in the Fc region of the antibodies described herein to increase resistance to host proteases, as described in, e.g., U.S. Pat. No. 8,871,204, CA2,822,366, and US2013/0011386.

### Fully Bovine Recombinant Monoclonal IgG1 Antibodies

Also provided herein are fully bovine recombinant monoclonal IgG1 antibodies and related oral compositions. Such antibodies can be generated by immunizing cattle (calves or mature cows, steers or bulls) with an antigen of interest, using the methods described in Example 11. In certain embodiments, cattle are immunized with DNA encoding the antigen of interest. General methods for immunizing animals with genetic vaccines (e.g., DNA vaccines) are known in the art (see, e.g., U.S. Pat. No. 8,927,508, WO2003/012117, WO2003/048371, WO1997/040839).

In certain embodiments, cattle (e.g., Holstein breed) are injected (e.g., subcutaneously) with the antigen of interest formulated together with an adjuvant. Any art-recognized adjuvant can be used. Suitable adjuvants include, but are not limited to, Emulsigen-D, Carbigen, Quil A, and Seppic ISA. In a preferred embodiment, the adjuvant is Quil A.

In some embodiments, cattle are inoculated with antigen 1, 2, 3, 4, 5, or 6 times, or more, to induce a specific immune response. In some embodiments, inoculations can be performed in 1, 2, 3, 4, or 5 week intervals. In one embodiment, cattle are inoculated 4 times at 2-3 week intervals.

In one embodiment, the antigen for immunization is injected into the area drained by the target superficial lymph node (targeted for harvesting lymphocytes).

In some embodiments, cattle are immunized 1, 2, 3, 4, or 5 days prior to removal of the target lymph nodes for subsequent hybridoma production.

The optimal concentration of target antigen used in the immunizations can be readily determined by the skilled artisan.

In a preferred embodiment, the antigen is TNFα, which is formulated together with Quil A adjuvant.

Once lymph nodes (or the spleen) are harvested from immunized cattle, lymphocytes are isolated and fused with an immortalized cell line using a suitable fusing agent, e.g., polyethylene glycol (PEG), in order to form a hybridoma cell (see, e.g., Goding (1986) Monoclonal Antibodies: Principles and Practice, Academic Press, pp. 59-103). Other suitable methods for generating hybridomas are described, for example, in U.S. Pat. Nos. 5,026,646, 5,087,693, Tucker et al. (Hybridoma 1984; 3:171-6), Kennedy et al., Journal of General Virology 1988; 69:3023-32), Srikumaran et al. (Veterinary Immunology and Immunopathology 1984; 5:323-42), and Raybould et al. (American Journal of Veterinary Research 1985; 46:426-7), and Levings et al. (Veterinary Immunology and Immunopathology 2014; 159:58-73).

Immortalized cell lines may be transformed mammalian cells, particularly myeloma cells of rodent, bovine, and human origin. Rat or mouse myeloma cell lines may be used. The hybridoma cells may be cultured in a suitable culture medium that preferably contains one or more substances that inhibit the growth or survival of the unfused, immortalized cells, For example, it the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines suitable for use are those that fuse efficiently, support stable high level expression of antibody by the selected antibody-producing cells, and are sensitive to a medium such as HAT medium. Preferred immortalized cell lines are murine myeloma lines, which can be obtained, for instance, from the Salk Institute Cell Distribution Center, San Diego, Calif, and the American Type Culture Collection, Manassas, Va.

The medium in which the hybridoma cells are cultured can then be assayed for the presence of monoclonal antibodies directed against the target of interest (e.g., TNF). The binding specificity of monoclonal antibodies produced by the hybridoma cells can be determined by immunoprecipitation or by an in vitro binding assay, such as radioimmunoassay (RIA) or enzyme-linked immunosorbent assay (ELISA). Such techniques and assays are well known in the art. The binding affinity of the monoclonal antibody can be determined by using art-recognized assays, such as Scatchard analysis and Biacore surface plasmon resonance assays.

After the desired hybridoma cells are identified, the clones may be subcloned by limiting dilution procedures and grown by standard methods. Suitable culture media include, for example, Dulbecco's Modified Eagle's Medium and RPMI-1640 medium. Alternatively, the hybridoma cells may be grown in vivo as ascites in a mammal.

In certain embodiments, the hybridoma cells are screened for the secretion of target-specific mononclonal IgG1 antibodies. The antibody isotype can be determined using standard sequencing procedures known in the art.

The fully bovine recombinant monoclonal IgG1 antibodies secreted by the subclones may be isolated or purified from the culture medium or ascites fluid by conventional immunoglobulin purification procedures, such as, protein A-Sepharose, hydroxylapatite chromatography, gel electrophoresis, dialysis, or affinity chromatography.

The monoclonal antibodies purified from hybridoma culture or ascites fluid can also be made using standard recombinant DNA methods known in the art. DNA encoding the bovine monoclonal antibodies can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells, Other suitable host cells include fungal cells (Aspergillus), insect cells, yeast cells, bacterial cells, and other art-recognized host cells.

In certain embodiments, fully bovine recombinant monoclonal IgG1 antibodies are generated through art-recognized single B cell sequencing and cloning methods, as described in Example 14. See also, e.g., US2011/0312505, US2012/030855, Murugan et al., Eur J Immunol 2015; 45:2698; 700, Liao et al., J Virol Methods 2009; 158:171-9; Busse et al., Eur J Immunol 2014; 44:597-603, the contents of all of which are herein incorporated by reference.

In one embodiment, the fully bovine recombinant monoclonal IgG1 antibodies are formulated with a preservative. In another embodiment, the fully bovine recombinant monoclonal IgG1 antibodies are lyophilized.

### Nucleic Acid Molecules

Also provided herein are nucleic acid molecules that encode the bovinized and fully bovine recombinant monoclonal IgG1 antibodies described herein. Once an antibody having increased resistance to proteases is identified, the coding nucleic acid sequence can be identified and isolated using art-recognized techniques.

The nucleic acids may be present in whole cells (e.g., hybridomas, ascites fluid, stable cell lines), in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or

RNA and may or may not contain intronic sequences. In a certain embodiments, the nucleic acid is a cDNA molecule.

Nucleic acids described herein can be obtained using standard molecular biology techniques. For antibodies expressed by hybridomas, cDNAs encoding the light and heavy chains of the antibody made by the hybridoma can be obtained by standard PCR amplification or cDNA cloning techniques.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region or VL chain can be converted to a full-length heavy chain or light chain gene by operatively linking the VH-encoding DNA or VL-encoding DNA to another DNA molecule encoding heavy chain or light chain constant regions. Nucleotide sequences for these regions are known in the art, and DNA fragments encompassing these regions can be obtained by standard PCR amplification. Also provided are nucleic acid molecules with conservative substitutions (i.e., substitutions that do not alter the resulting amino acid sequence upon translation of nucleic acid molecule), e.g., for codon optimization.

### Methods of Production

Also provided herein are host cell expression systems for producing the bovinized and fully bovine recombinant monoclonal antibodies described herein. Such host cell expression systems may be engineered to comprise a recombinant nucleic acid molecule encoding the bovinized antibodies or bovine monoclonal antibodies described herein, operatively linked to an art-recognized regulatory sequences, which include promoters, enhancers, and other expression control elements (e.g., polyadenylation signals) that control the transcription or translation of the antibody chain genes. Exemplary regulatory sequences are described, for example, in Goeddel (Gene Expression Technology. Methods in Enzymology 185, Academic Press, San Diego, CA (1990)). It will be appreciated by those skilled in the art that the design of the expression vector, including the selection of regulatory sequences, may depend on factors such as the choice of host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, recombinant expression vectors may include additional sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.), and allows for the establishment of stable cell lines (which can be stored for later use). Typically, the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

To express heavy and light chains of antibodies, expression vector(s) encoding the heavy and light chains is transfected into a host cell using art-recognized techniques. Transfection, as used herein, is intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

In general, any type of cultured cell line can be used as a background to engineer the host cell lines of the present invention including but not limited to CHO cells (e.g., dhfr-CHO cells used with a DHFR selectable marker), BHK cells, NS0 myeloma cells, COS cells, SP2/0 cells, YO myeloma cells, P3×63 mouse myeloma cells, PER cells, PER.C6 cells or hybridoma cells, yeast cells (e.g., Pichia pastoris, S. cerevisiae), bacterial cells (e.g., E. coli), fungal cells (e.g., Aspergillus niger, Aspergillus niger var. awamori), insect cells (SF9, SF21, High Five™), or plant cells (e.g., tobacco). In certain embodiments, host cell systems capable of glycosylating the recombinantly produced antibody are used. In further embodiments, these host cells are used for large-scale production of antibodies. Methods for large-scale production of antibodies are also known in the art (and are described in Example 19), e.g., in Aspergillus (Ward et al., Appl Environ Microb 2004; 70:2567-576), yeast (e.g., Pichia pastoris, S. cerevisiae), mammalian cells (Sinacore et al., Biotechnol Bioeng 1996; 52: 518-28; Li et al. mAbs 2010; 2:466-77; Wurm et al., Nat Biotechnol 2004; 22:1393-8; Birch et al., Adv Drug Delivery Rev 2006; 58:671-85; Jayapal et al., Chem Eng Prog 2007; 103:40-7; Mammalian Cell Culture for Biopharmacuetical Production. Chapter 12 of Manual of Industrial Microbiology and Biotechnology, 3 rd Edition. pp. 157-178 (2010)), and insect cells (Liang et al., J Immunol Methods 2001; 247; 119-30; Tan and Lam, Biotechnol Appl Biochem 1999; 30:59-64; U.S. Pat. No. 7,795,015). In certain embodiments, where purity of the antibody is not absolutely essential, e.g., for oral administration, fungal cells (e.g., Aspergillus species) are suitable for rapid gram-scale production of antibodies.

For methods of producing the antibodies described herein, stable expression is generally preferred to transient expression because it typically achieves more reproducible results and also is more amenable to large scale production. Rather than using expression vectors which contain viral origins of replication, host cells can be transformed with the respective coding nucleic acids controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then switched to a selective media. The selectable marker in the recombinant plasmid confers resistance to the selection and allows selection of cells which have stably integrated the plasmid into their chromosomes and grow to form foci which in turn can be cloned and expanded into cell lines.

When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Compositions

Provided herein are compositions (e.g., oral pharmaceutical compositions) comprising the bovinized and fully bovine recombinant monoclonal antibodies described herein.

Pharmaceutical compositions described herein comprise a therapeutically effective amount of a bovinized and/or fully bovine recombinant monoclonal antibody, optionally formulated together with one or more pharmaceutically acceptable carriers or excipients.

Accordingly, in some embodiments, provided herein are pharmaceutical compositions comprising or consisting essentially of bovinized and/or fully bovine recombinant monoclonal antibodies, and optionally a carrier (e.g., a pharmaceutically acceptable carrier and/or preservative). In other embodiments, the composition is substantially free, e.g., at least 90%, 95%, or 99% free, from other (i.e., non-bovine IgG1 or bovinized) antibodies. In particular embodiments, the pharmaceutical composition is formulated for oral administration.

For treating disorders of the oral cavity, the compositions can be delivered in a mouthwash, rinse, paste, gel, or other suitable formulation. The antibodies described herein can be delivered using formulations designed to increase the contact between the active antibody and the mucosal surface, such as buccal patches, buccal tape, mucoadhesive films, sublingual tablets, lozenges, wafers, chewable tablets, quick or fast dissolving tablets, effervescent tablets, or a buccal or sublingual solid. For treating disorders of the digestive tract, the antibody can be delivered by oral ingestion in the form of a capsule, tablet, liquid formulation or similar form designed to introduce drug to the digestive tract. Alternatively, antibody may be administered by suppository or enema for delivery to the lower digestive tract. Such formulations are well known to those skilled in the art. In certain embodiments, the antibody is administered prior to and/or concurrently with hydrolase inhibitors, antibiotics, and/or protease inhibitors.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are water, sterile water, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical composition may also include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound. In one embodiment, compositions for rectal administration are in the form of an enema.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Although the antibodies described herein exhibit enhanced stability to intestinal degradation, it may be desirable under some conditions to provide additional levels of protection against intestinal degradation. If this is desired, there are many options for enteric coating (see for example U.S. Pat. Nos. 4,330,338 and 4,518,433). In one embodiment, enteric coatings take advantage of the post-intestinal change in pH to dissolve a film coating and release the active ingredient. Coatings and formulations have been developed to deliver protein therapeutics to the small intestine and these approaches could be adapted for the delivery of an antibody of the invention. For example, an enteric-coated form of insulin has been developed for oral delivery (Toorisaka et al., J Control Release 2005; 107:91-6). Additional options for enteric coating are described in, e.g., U.S. Pat. Nos. 5,225,202 and 6,306,900, and US2008/0020041. In addition, the solid dosage forms of tablets, dragees, capsules, pills, and granules can be prepared with other coatings and shells well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. In a preferred embodiment, the antibody is formulated in enterically-coated microparticles delivered as a liquid suspension. In another preferred embodiment, the antibody is formulated in enterically-coated microparticles delivered as a capsule.

The compositions (e.g., oral pharmaceutical compositions) described herein may also contain wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions.

In some embodiments, the pharmaceutical compositions comprising the antibodies described herein also include a preservative.

Effective doses will vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the timing of delivery of the compound relative to food intake; the duration of the treatment; drugs used in combination or contemporaneously with the specific compound employed; and like factors well known in the medical arts.

Particular embodiments of the present invention involve administering a pharmaceutical composition comprising an antibody of the invention at a dosage of from about 1 mg per day to about 1 g/day, more preferably from about 10 mg/day to about 500 mg/day, and most preferably from about 20 mg/day to about 100 mg/day, to a subject. In one embodiment, a polyclonal antibody preparation is administered at a dosage of antibody from about 100 mg to about 50 g/day, more preferably from about 500 mg/day to about 10 g/day, and most preferably from about 1 g/day to about 5 g/day, to a subject, wherein the polyclonal antibody preparation has not been enriched for antibodies specific for the target antigen.

Treatment regimens include administering an antibody composition of the invention one time per day, two times per day, or three or more times per day, to treat a medical disorder disclosed herein. In one embodiment, an antibody composition of the invention is administered four times per day, 6 times per day or 8 times per day to treat a medical disorder disclosed herein. In one embodiment, an antibody composition of the invention is administered one time per week, two times per week, or three or more times per week, to treat a medical disorder disclosed herein.

The methods and compositions of the invention include the use of an antibody of the invention in combination with one or more additional therapeutic agents useful in treating the condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which an antibody of the invention is administered at least once during a course of treatment that involves administering at least one other therapeutic agent to the patient.

An antibody to be used for in vivo administration must be sterile. This is readily accomplished by filtration through sterile filtration membranes, prior to or following lyophilization and reconstitution. The formulation ordinarily will be stored in lyophilized form, as spray dried particles or in solution.

### Methods of Use

Topical administration of antibodies to the digestive tract is challenging because the digestive tract degrades and digests the topically applied antibodies. Enzymes in the oral cavity, primarily derived from commensal and pathogenic bacteria living within the oral cavity, degrade antibody in the oral cavity. In the stomach, the low pH and the protease pepsin degrade ingested immunoglobulin. In the small intestine, the enzymes trypsin and chymotrypsin, among others, degrade ingested antibody. In the large intestine, bacterially-derived proteases degrade ingested antibody. Antibodies with improved stability in the oral cavity and improved stability to gastric and intestinal digestion (e.g., the bovinized and fully bovine recombinant monoclonal antibodies described herein) have numerous in vivo applications, for example, in the treatment of diseases involving the digestive tract (e.g., inflammatory bowel disease).

The antibodies described herein may be used in the oral cavity for the prevention of dental caries and for the treatment or prevention of periodontal disease as described in U.S. Pat. Nos. 5,759,544; 4,689,221; 4,324,782; 4,693,888; 4,725,428; 6,143,330; 5,240,704 and 5,352,446, for the control of microorganisms, including bacteria, protozoa, parasites, viruses, and fungi, or for the control of inflammation through the use of antibodies specific for cytokines or chemokines, or receptors for cytokines or chemokines. Bovinized and fully bovine recombinant monoclonal antibodies used in the oral cavity may be specific for receptors or other antigens expressed on the apical surface of the oral cavity, against receptors or other antigens expressed on the basolateral surface of the mucosal barrier of the oral cavity, or against receptors or other antigens expressed in the mucosa, submucosa, or any other region of the body accessible to topically applied antibody. Such antibodies may be used for the treatment of infections of the oral cavity or diseases of the oral cavity, including but not limited to mucositis, cancers of the oral cavity, nicotinic stomatitis, leukoplakia, hairy tongue, recurrent aphthous stomatitis, geographic tongue, denture stomatitis, gastroesophageal reflux, eosinophilic esophagitis, and lichen planus. The antibodies may also be applied topically to the oral cavity as a diagnostic reagent as described in U.S. Pat. No. 7,175,430.

Many of the proteases present in the oral cavity are of bacterial origin. In one embodiment, a topical antibiotic is administered to the oral cavity prior to topical administration of antibody. In another embodiment, protease inhibitors are administered to the oral cavity prior to and/or concurrently with topical administration of antibody. Microbes in the oral cavity also produce hydrolases that remove carbohydrate from antibody, thus making it more susceptible to proteolytic degradation. Accordingly, in one embodiment, hydrolase inhibitors are administered to the oral cavity prior to and/or concurrently with topical administration of antibody. Antibiotics, protease inhibitors, and hydrolase inhibitors may be given in combination.

The antibodies described herein may be used in the GI tract for the treatment or prevention of diseases, including but not limited to bacterial, viral, or parasitic infections of the gastrointestinal tract, cancers of the gastrointestinal tract, and inflammation of the gastrointestinal tract as a result of injury, surgery, radiation, infection, or autoimmune disease.

The antibodies described herein are useful in the modulation of apical receptors in the digestive tract, including nutrient receptors, nutrient transporters, pattern recognition receptors, chemokine receptors, cytokine receptors, bile salt transporters, inorganic ion transporters, mineral transporters, peptidases, saccharases, and growth factor receptors.

The antibodies described herein are useful in the treatment or prevention of food allergies or intolerances, including celiac disease. In one embodiment, the antibody is specific for gluten or gluten derived peptide and is used to treat celiac disease.

The antibodies described herein are useful in modulating the function of receptors, cytokines, chemokines, or similar mediators expressed in the lumen of the digestive tract or, in the case of a disease or condition that renders the digestive tract permeable to topically applied antibodies, in modulating the function of receptors, cytokines, chemokines, or similar mediators expressed in the portions of the body below the mucosal barrier that are accessible to the antibody. The antibodies described herein are also useful in the treatment of immunodeficiency.

The antibodies described herein have minimal activity outside of the digestive tract and minimize the induction of a neutralizing immune response. The therapeutic compositions comprise antibodies that are delivered topically to the luminal face of the digestive tract. The antibodies described herein may be administered topically to the digestive tract by, for example, oral administration, rectal administration, and all forms of administration to the oral cavity such as by buccal, mucoadhesive films and the like. The antibodies may cross the mucosal barrier of the digestive tract to enter the submucosal space to interact with their targets, but do not enter the systemic circulation at levels sufficient to be clinically relevant.

In certain embodiments, the antibodies described herein bind to antigens associated with disease pathology or the treatment of disease. For example, the antibody compositions described herein may be directed at biological targets expressed on or near the luminal surface of the digestive tract as well as below the mucosal barrier such as on the basal side of the epithelium, targets expressed in the submucosa, target expressed in the lateral intercellular space, and targets expressed in the lamina propria.

In some embodiments, antibody compositions described herein cross the mucosal barrier of a patient as a result of pre-existing damage to the mucosal barrier. In one embodiment, the mucosal barrier of the digestive tract may be breached or compromised through mechanical trauma, including but not limited to dental and oral wounds, esophageal wounds, or surgically induced trauma due to partial gut resection, jejunostomy, ileostomy, colostomy or other surgical procedures. The mucosal barrier of the digestive tract may also be breached by ischemia or reperfusion injury. The mucosal barrier of the digestive tract may also be breached by damage caused by cancer chemotherapy, cancer radiation therapy, or high dose radiation exposure outside of a therapeutic setting. The mucosal barrier of the digestive tract may be breached or compromised through gross inflammation and/or ulceration, including but not limited to periodontal disease, aphthous stomatitis, bacterial, viral, fungal or parasitic infections of the digestive tract, peptic ulcers, ulcers associated with stress or H. pylori infection, damage caused by esophageal reflux, inflammatory bowel disease, damage caused by cancer of the digestive tract, food intolerance, including celiac disease, or ulcers induced by non-steroidal anti-inflammatory drugs (NSAIDs) or other ingested or systemically delivered drugs. The mucosal barrier of the digestive tract may also be breached by genetically-determined pre-disposition to increased intestinal permeability.

In some embodiments, the antibodies described herein cross the mucosal barrier as a result of specific aspects of the formulation that facilitate the transit of antibody across the mucosal barrier. Exemplary permeation enhancers include but are not limited to chitosan, poly-L-arginine and Carbopol.

In some embodiments, the antibodies described herein are loaded onto inflammation-targeting hydrogels (IT-hydrogel) to reduce systemic exposure and target antibodies to the inflamed colon of patients with inflammatory bowel disease (Crohn's disease and ulcerative colitis) (see Zhang et al., Sci Transl Med 2015; 7:300ra128). Also suitable for targeting the antibodies described herein to inflamed tissue in the colon are nanodelivery systems, for example, those described in Hua et al., Nanomedicine: Nanotechnology, Biology, and Medicine 2015; 11:1117-32.

In some embodiments, the antibodies described herein are specific for target antigens such as cytokines that regulate inflammation, including but not limited to TNF, TNF-kappa, IFN-gamma, IL-1 beta, IL-2, IL-6, IL-12, IL-13, IL-15, IL-17, IL-18, IL-21, IL-23, IL27, IL-32, IL-33, and IL-35. In other embodiments, the antibodies described herein are specific for target antigens that are enteric neurotransmitters or their receptors or transporters expressed below the mucosal barrier of the digestive tract, including receptors for serotonin that are expressed in the gut (5-HT1A, 5-HT1B/B, 5-HT2A, 5-HT2B, 5-HT3, 5-HT4, 5-HT7, 5-HT1P). In further embodiments, the antibodies described herein re specific for target antigens that are peptides that regulate food intake or the receptors for such peptides. Such peptides include but are not limited to CCK, GLP1, GIP, oxyntomodulin, PYY3-36, enterostatin, APOAIV, PP, amylin, GRP and NMB, gastric leptin, and ghrelin. In other embodiments, the antibodies described herein are specific for target antigens that are biological targets that enhance wound healing, that alter the function of tight junctions such as occludin, claudins, junctional adhesion molecule, ZO-1, E-cadherin, coxackie adenovirus receptor, and serine proteases such as elastase that are involved in the release of claudins.

In one embodiment, the antibodies described herein are specific for EGFR on colorectal cancer cells.

In another embodiment, the antibodies described herein are specific for Toll-like receptors expressed on the basolateral face of mucosal epithelial cells, and are applied to the mucosa of the digestive tract of a patient with an intestinal inflammatory disease.

In one embodiment, the antibodies described herein are specific for target antigens that are apical intestinal receptors. “Apical intestinal receptors” as used herein are endogenous transmembrane proteins, expressed in the cell membrane of cells facing the luminal side of the intestinal tract. Classes of apical intestinal receptors include but are not limited to: nutrient receptors and transporters (including sugar receptors and transporters, taste receptors, amino acid transporters, and free fatty acid receptors); pattern recognition receptors (including the Toll-like receptors); chemokine and cytokine receptors; bile salt transporters; transporters for calcium iron, and other ions and minerals; peptidases; disaccharidases; growth factor receptors (including epidermal growth factor receptor) and proteins expressed on the surface of cancerous cells in the GI tract. Apical intestinal receptors may be expressed in the stomach, the small intestine or the colon.

In a preferred embodiment, the antibody is specific for tumor necrosis factor-alpha (TNF). In some embodiments, compositions comprising such anti-TNF antibodies are suitable for use in the treatment of inflammation, and particularly inflammatory bowel disease, which includes Crohn's disease and ulcerative colitis.

In some embodiments, compositions comprising anti-TNF antibodies are suitable for use in the treatment of oral or intestinal mucositis. The mucositis may, for example, be caused by radiation therapy, chemotherapy or any combination thereof. Mucositis may also be caused by exposure to high doses of radiation, including total body irradiation, outside of the context of radiation therapy. Chemotherapeutic agents which may induce mucositis when used alone or in combination include, but are not limited to, platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide, and doxorubicin. Additional agents include inhibitors of mTOR (mammalian target of rapamycin), including but not limited to rapamycin, everolimus, temsirolimus, and deforolimus. In one embodiment, compositions comprising the anti-TNF antibodies are suitable for use in the treatment of recurrent aphthous stomatitis, eosinophilic esophagitis, eosinophilic gastritis, and other conditions involving hypereosinophilic activity in a part of the gastrointestinal tract. The compositions may be administered topically, for example, to the oral cavity to treat oral mucositis and aphthous stomatitis, or orally or rectally to the digestive tract, for example, to treat intestinal mucositis.

In one embodiment, for the treatment of aphthous stomatitis (RAS), the antibodies described herein can be administered at the earliest manifestation of an ulcer. Alternatively, the antibodies can be administered on a regular basis throughout the course of manifestation of the ulcer. Alternatively, the antibodies can be administered on a regular basis to prevent the recurrence of ulcer formation. In a preferred embodiment, provided herein is a method of treating recurrent aphthous stomatitis (RAS) in a patient comprising administering to the patient by topical application to the oral cavity a therapeutically effective amount of a composition comprising an antibody specific for TNF (an anti-TNF antibody). In one embodiment, the anti-TNF antibody is a bovinized or fully bovine recombinant monoclonal antibody, as described herein. In one embodiment, the anti-TNF antibody is an antibody with enhanced mucosal permeability. In another embodiment anti-TNF antibody is administered topically to the oral cavity as described herein.

In some embodiments, the anti-TNF antibodies are co-administered with additional therapeutic agents to treat, e.g., inflammatory bowel disease. Exemplary agents suitable for co-administration include, but are not limited to, oral steriods, IFN-6, budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1βmonoclonal antibodies; anti-IL-6 monoclonal antibodies; growth factors; elastase inhibitors; pyridinyl-imidazole compounds; CDP-571/BAY-10-3356 (humanized anti-TNF antibody; Celltech/Bayer); 75 kdTNFR-IgG (75 kD TNF receptor-IgG fusion protein; Immunex; see e.g., Arthritis & Rheumatism 1994; 37:5295; J Invest Med 1996; 44:235A); 55 kdTNFR-IgG (55 kD TNF receptor-IgG fusion protein; Hoffmann-LaRoche); interleukin-10 (SCH 52000; Schering Plough); IL-4; IL-10 and/or IL-4 agonists (e.g., agonist antibodies); interleukin-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of Platelet Activating Factor (PAF); ciprofloxacin; and lignocaine.

Additional potential co-therapeutic agents suitable for treating TNF modulated disease (e.g. IBD) are commercially available or are currently in clinical development and include the following: 5-ASA (generic); MMX Mesalazine (Cosmo); MMX Budesonide (Cosmo); MMX LMW Heparin (Cosmo); ER Mesalazine (Salix); Azathioprine (generic); 6-mercaptopurine; Infliximab (Centocor, J&J); Adalimumab (Abbott); Certolizumab pegol (UCB); Atrosab (BalioPharma); Natalizumab (Elan); Golimumab (Centocor J&J); Dersalazine (Palau); HMPL-004 (Hutchinson Medi Pharma); Ozoralizumab (Ablynx); TNF-α Kinoid (Neovacs); Apilimod (Synta); Ustekinumab (Centocor J&J); Briakinumab (Abbott); SCH-900222 (Schering Plough); FM202 and FM303; MP-196; Basiliximab (Cerimon); Daclizumab (Roche); Fontolizumab (PDL); C326 (Avidia); Sirukumab (Centocor J&J); Olokizumab (UCB); Sarilumab (Centocor J&J); BMS-945429 (Alder); Tocilizumab (Chugai); Anrukinzumab (Wyeth); QAX567 (Novartis); GSK1070806 (GSK); PF-05230900 (Pfizer) Vidofludimus (4SC); Tofactinib (Pfizer); Visilizumab (PDL); Rituximab (Genentech); Abatacept (BMS); Filgrastim (Amgen); Sargramostim (Immunex, Amgen); Vedolizumab (Takeda); Etrolizumab (Genentech); AJM-300 (Ajinimoto); ASP-2002 (Mitsubishi); Alicaforsen (Isis); PF-547659 (Pfizer); CCX282 (GSK1605786); Remestemcel-L (Osiris); PDA-001 (Celgene); OvaSave (TxCell); Secukinumab; MDX-1100 (Medarex); Tetomilast (Otsuka); LT-02 (Lipid Therapeutics); ozanimod (Receptos/Celgene); apremilast (Celgene); bertilumumab (Immune Sciences); abrilumab (AstraZeneca); ABT-494 (AbbVie); B1655066 (Boehringer Ingelheim); Mongersen (Celgene); MT-1303 (Mitsubishi Tanabe); and PF-00547659 (Pfizer).

In one embodiment, antibodies delivered to the digestive tract that are specific for soluble cytokines reduce levels of those cytokines in the digestive tract, but not in the systemic circulation. Levels of cytokine can be determined by direct measurement of the cytokine or by analysis of a surrogate marker that responds to the cytokine. In one aspect, antibodies delivered to the digestive tract that are specific for soluble cytokines reduce levels of those cytokines in both the digestive tract and systemic circulation.

In one embodiment, antibodies delivered to the digestive tract that have clinical benefit do not induce an antibody response to the administered antibody that is sufficient to inhibit the response to subsequent doses of the antibody or to cause an injurious response to subsequent doses of the antibody.

In another embodiment, the lack of an induced antibody response is seen following maintenance therapy. In a further embodiment, the lack of an induced antibody response is seen following episodic dosing. The antibody response can be measured by direct measurement of antibody specific for the therapeutic antibody or by assessment of the physiological response to repeated doses of the therapeutic antibody.

In a preferred embodiment, fewer than 2% of patients develop antibodies to the therapeutic antibodies of the invention after exposure to 3 or more doses of therapeutic antibody. In another preferred embodiment, administration of 3 or more doses of the antibody does not lower the efficacy of the antibody. In one embodiment, the efficacy of the antibody is not diminished after administration of 1, 2, 3 or more doses over a period of about 1 month from the date of first administration of the antibody, and preferably over a period of about 6 months from the date of the first administration of the antibody, more preferably over a period of about 1 year from first administration of the antibody, and even more preferably over a period of about 10 years from first administration of the antibody.

Measurements of the antibody response to the therapeutic antibody (TA) can be accomplished using standard assays as are known in the prior art, such as RIA and solid phase RIA. Assays for assessing the antibody response to a therapeutic antibody can be adapted to the specific situation and the methods and considerations are well understood (Gorovits, The AAPS Journal, Vol. 11, No. 1, March 2009). For assaying antibody responses to the antibodies described herein, a direct comparison of the pretreatment and post-treatment sample results for a given patient is preferred. A patient is defined as having an induced antibody response against the therapeutic antibody if the levels of anti-TA antibody significantly increase during the course of treatment with the therapeutic antibody. For therapeutic antibodies where preexisting antibody responses are not detected, a patient is defined as having an induced antibody response against the therapeutic antibody if the antibody response is greater than two-fold above background.

In addition to the digestive tract, the antibodies described herein may also be applied to other tissues with mucosal barriers, including the urogenital system and the respiratory system. The antibodies described herein may also be applied to other tissues with an epithelial system, including the eye and the skin.

### Kits

Also within the scope of the invention are kits comprising the antibody compositions (pharmaceutical compositions) described herein (e.g., bovinized antibodies, fully bovine recombinant monoclonal antibodies, bispecific antibodies, or immunoconjugates) and instructions for use. In some embodiments, the kit further contains at least one additional reagent. Kits typically include a label indicating the intended use of the contents of the kit. The term label includes any writing, or recorded material supplied on or with the kit, or which otherwise accompanies the kit.

The present disclosure is further illustrated by the following examples, which should not be construed as further limiting. The contents of all figures and all references, Genbank sequences, and patents and published patent applications cited throughout this application are expressly incorporated herein by reference.

### EXAMPLES

### Example 1—Bovine Colostral Antibodies are Resistant to Intestinal Digestion

AVX-470 is a bovine colostral polyclonal antibody that binds to human tumor necrosis factor (TNF). Patients with ulcerative colitis (UC) were administered AVX-470 in a delayed-release, enteric-coated capsule formulation to evaluate the presence of AVX-470 in stool using a sandwich ELISA assay specific for bovine immunoglobulin (Ig).

Approximately 1 g of fecal sample (0.8 to 1.2 g) was added to 8 mL Extraction Buffer Solution (PBS with a protease inhibitor cocktail). Bovine Ig concentrations in the samples were analyzed using a colorimetric sandwich ELISA. Microtiter plates were coated with a commercially sourced polyclonal sheep anti-bovine IgG (H+L) antibody (Bethyl Laboratories, cat #A10-115A) as the capture antibody and incubated for 1.5 hours. The plate was washed and blocked with PBS-Tween 0.05% (PBST). After blocking, wells were washed, and stool samples at two different dilutions and bovine Ig standards from Bovine Reference Serum (BRS) were added in triplicate to the plate. All samples were tested with no further dilution after homogenization and at a 1:5 dilution after homogenization. After an hour, the plate was washed, and a polyclonal sheep anti-bovine Ig (H+L) antibody coupled to horseradish peroxidase (HRP) (Bethyl Laboratories, cat #A10-115P) was added for another hour-long incubation. The plate was washed again, and TMB chromogenic substrate was added to all wells. The reaction was stopped after fifteen minutes by adding diluted sulfuric acid. In the presence of bovine Ig, the wells turned blue. The optical densities (OD) were read on an Epoch Biotek Micro-plate reader with Gen5 software (Winooski, VT) at a 450 nm wavelength.

Bovine Ig was detected in six out of 32 (19%) pre-dose stool samples. An increased number of stool samples collected from patients at the end of the study, a total of 15 out of 32 (47%), were positive for bovine Ig after 28 days of dosing. Three of eight (38%) patients who received low dose AVX-470 (0.2 g/d) were positive for bovine Ig in stool, which was equivalent to the number of positive samples in the placebo group. In patients who received 1.6 g/d, six of ten (60%) stool samples were positive for bovine Ig. In the highest dose group (3.5 g/d) four of the six (67%) stool samples had detectable bovine Ig. Thus, there was a dose-dependent increase in the frequency of detectable bovine Ig in stool.

All samples were analyzed for TNF binding by direct ELISA. ELISA plates were coated with commercially available recombinant human TNF (Cell Sciences: Canton, MA, cat #CRT100C) at a concentration of 100 ng/well. After a one hour incubation at room temperature, ELISA plates were washed and blocked with Superblock (ThermoFisher, cat #37515). After an additional wash step, serial dilutions of AVX-470 (used as a positive control) or stool samples diluted at both 1:5 and 1:25 were added to ELISA plates. AVX-470 and stool samples were incubated at room temperature for one hour. After washing the plate, a polyclonal sheep anti-bovine Ig (H+L) antibody coupled to horseradish peroxidase (HRP) (Bethyl Laboratories, cat #A10-115P) was added for another hour-long incubation. After a final wash step, TMB chromogenic substrate was added to all wells. The reaction was stopped after fifteen minutes by adding diluted sulfuric acid. The optical densities (OD) were read on an Epoch Biotek Micro-plate reader at a 450 nm wavelength and results were analyzed using the Gen5 software (Winooski, VT).

TNF binding activity was associated with bovine Ig was detected in an ELISA assay in the three stool samples with the highest levels of bovine Ig, and TNF binding activity was absent from all placebo and pre-dose samples. This demonstrates the presence of active AVX-470 in stool and confirms intestinal stability of AVX-470, and more generally that bovine colostral antibodies are resistant to intestinal digestion.

### Example 2—Pancreatin Digestion of AVX-470 Compared to Human IgG and Infliximab

The susceptibility of AVX-470 (a polyclonal bovine colostral antibody) to digestion by pancreatin was compared with that of human serum IgG1 (Athens Research & Technology cat #16-16-090707-1) and infliximab (a recombinant human-mouse chimeric antibody specific for human TNF).

Solutions for human IgG1, infliximab, and AVX-470 were diluted and 5 mg/mL pancreatin in simulated intestinal fluid (SIF) was prepared. AVX-470, Infliximab and Human IgG1 were added to tubes containing SIF and various points were selected to stop the reaction with Protease Inhibitor (Lot #P8340-5 mL, Sigma Aldrich). The pancreatin:substrate ratio was 10:1.

For reducing gels, 7.35 μL mixture of SDS (4×) (5.25 μL)+DTT (10×) (2.1 μL) was added. Samples were heat treated for 10 minutes at 95° C. Gels were run at 180 volts for 50 minutes, then stained with the EZBlue Commassie kit (Sigma-Aldrich). The two most prominent protein bands in these samples have apparent molecular weights of 50 kDa (corresponding to the IgG heavy chain) and 23 kDa (IgG light chain). The intensity of the 50 kDa (heavy chain) band was quantified with ImageJ software (NIH) and plotted as a function of incubation time.

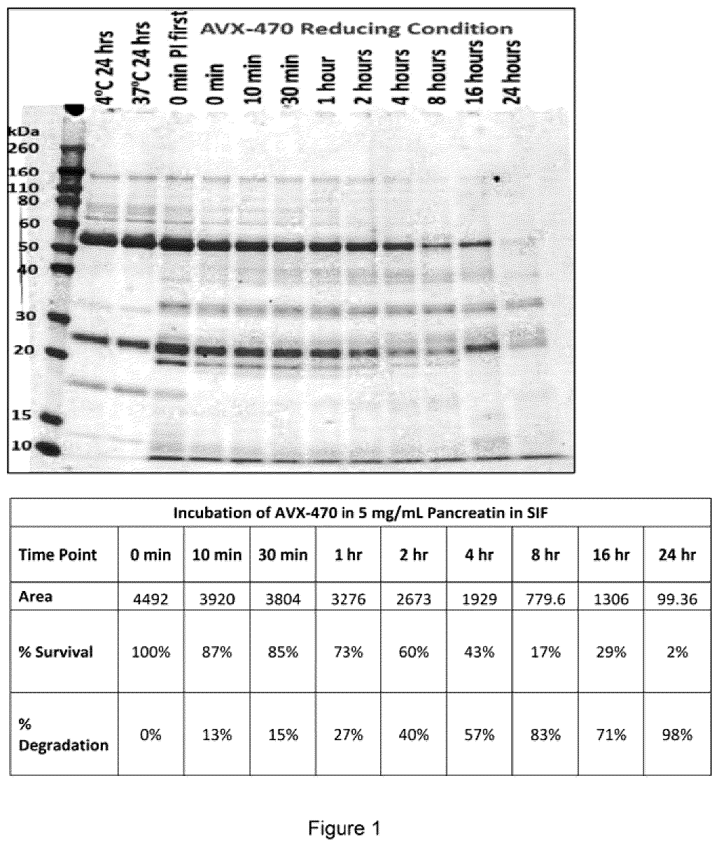

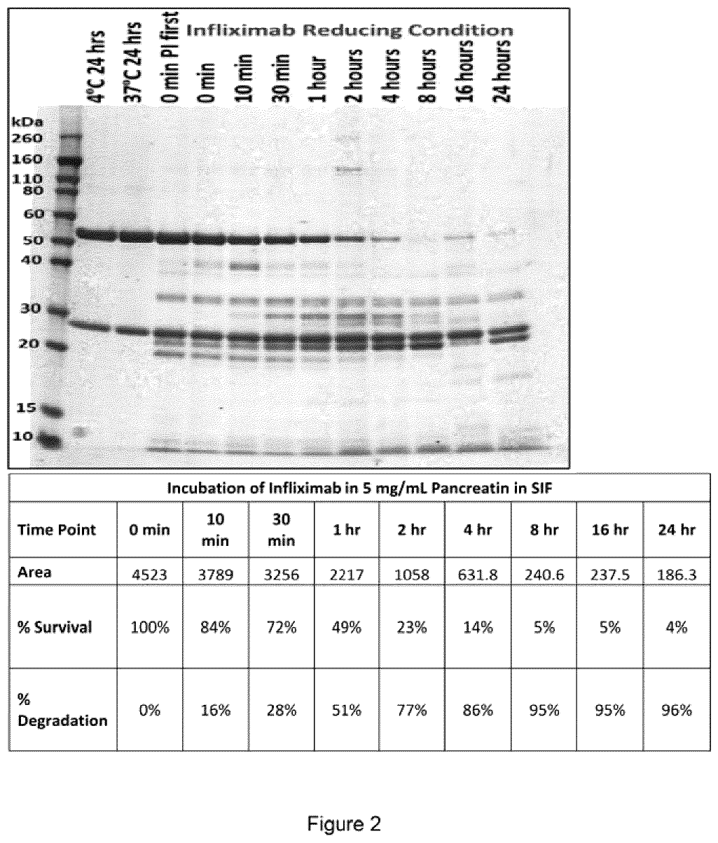

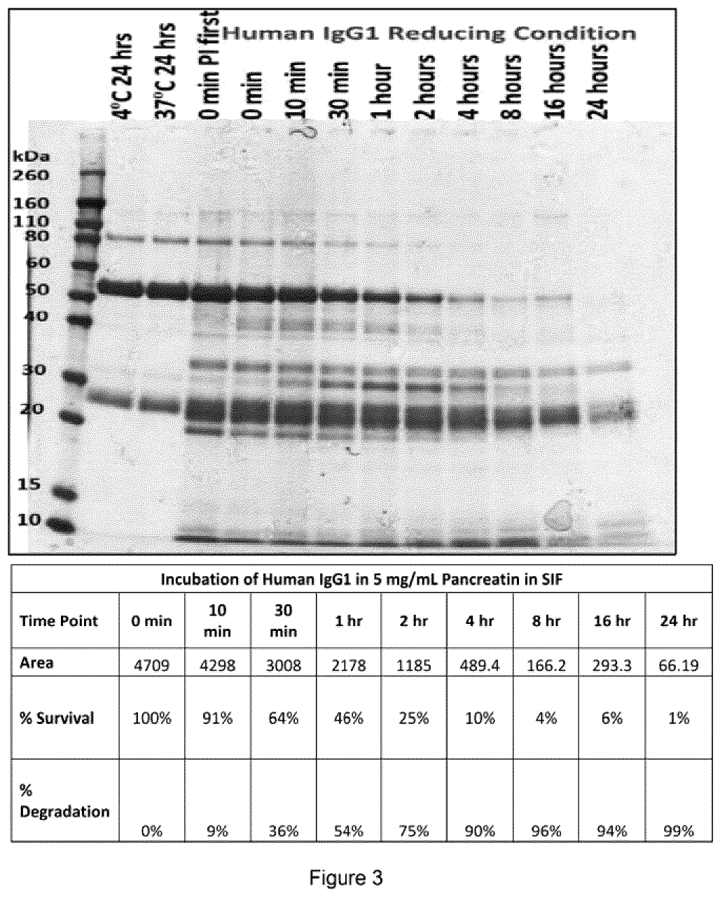

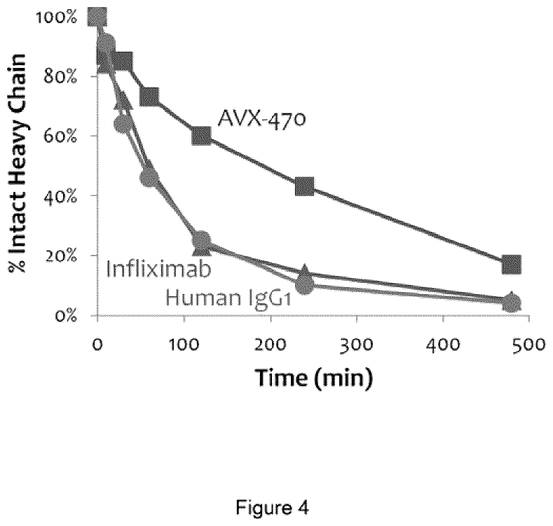

As shown in FIGS. 1-4, AVX-470 was more resistant to digestion with pancreatin compared to human IgG and infliximab.

### Antigen-Binding Analysis of Proteolysis Reactions by ELISA

The pancreatin digest samples described above were assessed for antigen-binding activity by ELISA. ELISA plates were coated with 10 μg/mL recombinant human TNF and blocked with SuperBlock. Samples were applied to selected wells at dilutions of either 1:10 and 1:20 (AVX-470) or 1:100,000 and 1:200,000 (infliximab) in PBS+0.05% polysorbate 20+2% SuperBlock. Reference standards of undigested AVX-470 or infliximab were added to separate wells. The samples and standards were allowed to bind to the plate for 1 hour. The plates were then washed, probed with either HRP-rabbit anti-bovine IgG (H+L) or HRP-mouse anti-human IgG secondary antibody. The plates were incubated for 1 hour, washed, and probed with TMB substrate to detect bound antibody.

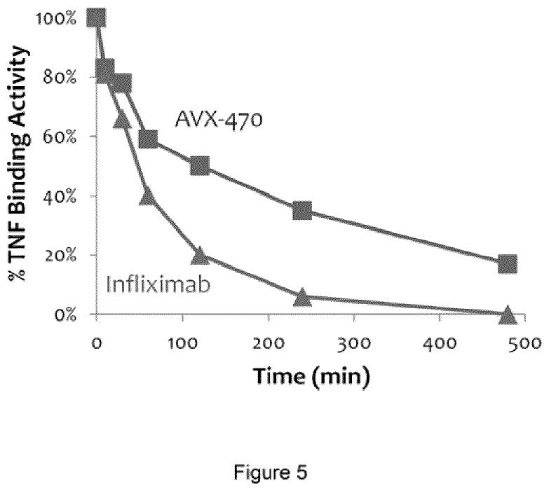

Compared to the TNF-binding activity of the colostral anti-TNF antibody (AVX-470), the TNF-binding activity of infliximab declined rapidly upon incubation with pancreatin (FIG. 5).

### Example 3—Pancreatin Digests of Serum, Milk, and Colostral Bovine IgG from the Same Cow

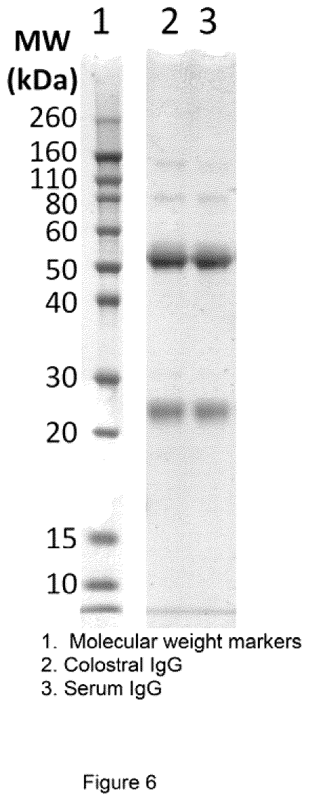

Results from Example 2 demonstrated that human IgG and inflixmab exhibit higher susceptibility to protease digestion than AVX-470, suggesting that molecular differences exist between these antibodies that restrict proteolysis of the bovine colostral antibody. To further determine the characteristics of colostral antibodies that may confer stability against proteases, a series of experiments were performed with bovine IgG from colostrum, milk, and serum. All IgG samples were purified by Protein G affinity chromatography from bodily fluids of cows that had been immunized with recombinant human TNF in the final trimester of pregnancy. Serum IgG was purified from a serum sample collected on the day of parturition. Colostral IgG was isolated from the first milking taken on the day of parturition. Milk IgG was purified from pooled milk samples taken 12-30 days post-parturition. Purified colostral IgG and serum IgG samples subjected to SDS-PAGE are shown in FIG. 6.

0.4 mg/mL IgG solutions were prepared in simulated intestinal fluid (SIF). Samples were pre-warmed to 37° C., then combined with porcine pancreatin as follows:

Mix: 1260 μL 0.4 mg/mL IgG
140 μL 10 mg/mL pancreatin in SIF
   At each time point, the reaction mixture was combined with protease inhibitor:
   Mix: 150 μL reaction mix
3 μL protease inhibitor cocktail
   The following time points were collected for each sample:

TABLE 9

| Aliquot | Incubation time |
|---|---|
| 1 | 1 minute |
| 2 | 22 minutes |
| 3 | 52 minutes |
| 4 | 1 hour, 47 minutes |
| 5 | 4 hours, 17 minutes |
| 6 | 10 hours, 53 minutes |
| 7 | 23 hours, 20 minutes |

Samples were prepared for SDS-PAGE analysis as follows:
SDS Load Mix: 528 μL water
330 μL 4×LDS buffer
132 μL 10×DTT

Mix: 10 μL sample
30 μL SDS Load Mix

Samples were heated to 95° C. for 15 minutes, then spun briefly to collect condensate at the top of each tube. 10 μL of each sample was loaded onto 12% acrylamide gels (Novex) and electrophoresed for 100 minutes at 120 V. The gels were stained with EZ-Blue following the kit instructions. The two most prominent protein bands in these samples have apparent molecular weights of 50 kDa (corresponding to the IgG heavy chain) and 23 kDa (IgG light chain). The intensity of the 50 kDa (heavy chain) band was quantified with ImageJ software (NIH) and plotted as a function of incubation time.

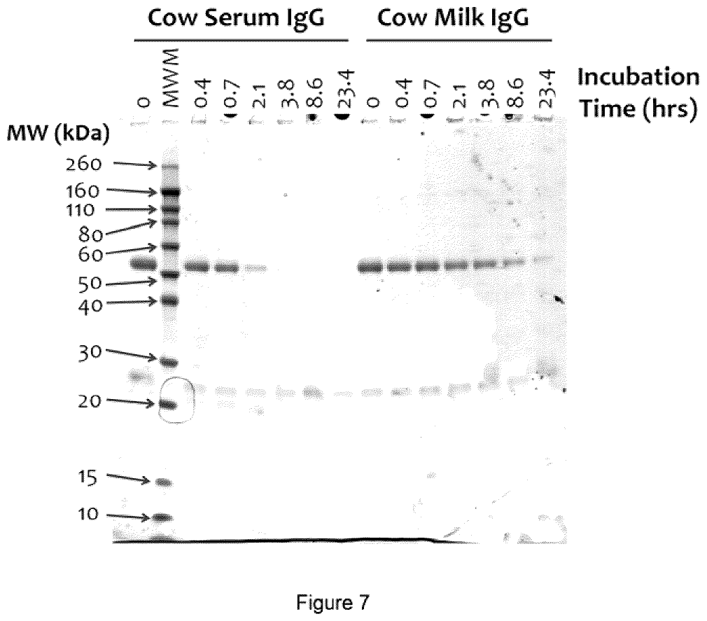

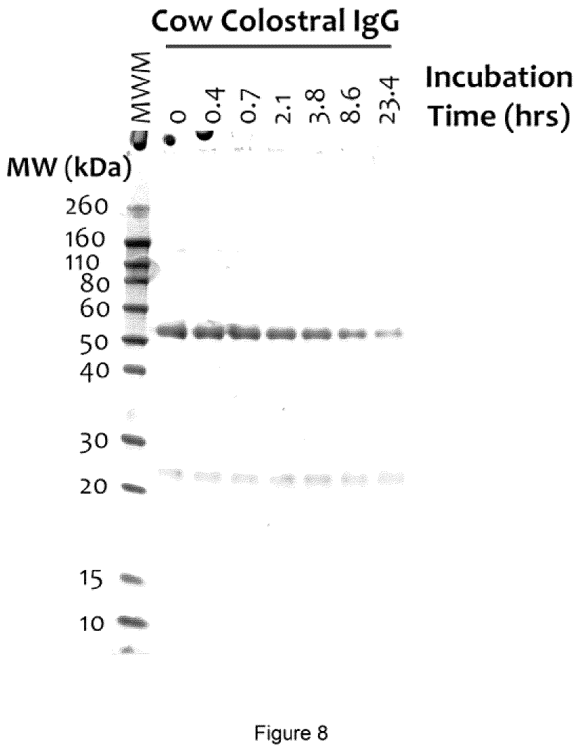

As shown in FIGS. 7-9, and Table 10, colostral IgG is the most stable against proteolysis. Milk IgG is degraded somewhat more rapidly, and serum IgG is proteolyzed significantly faster than colostral IgG and milk IgG.

TABLE 10

| IgG Source | t_{1/2} (hrs) |
|---|---|
| Colostrum | 57.8 |
| Milk | 12.4 |
| Serum | 2.3 |

This data suggests that a factor associated only with colostral antibodies is responsible for pancreatin resistance. Potential factors that could, in theory, confer this resistance include glycosylation, a stabilizing entity present in the colostrum, and bovine immunoglobulin isotype.

### Example 4—Carbohydrate Profiles of Serum, Milk, and Colostral Antibodies

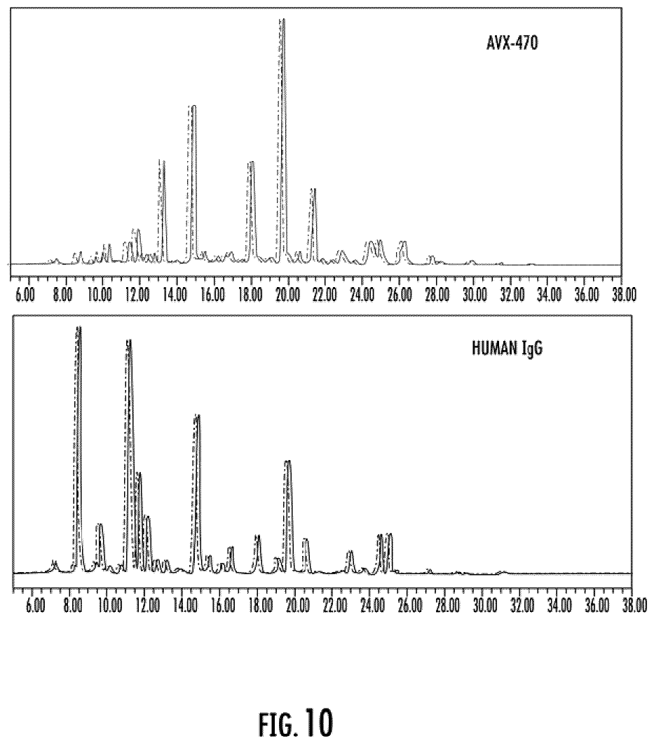

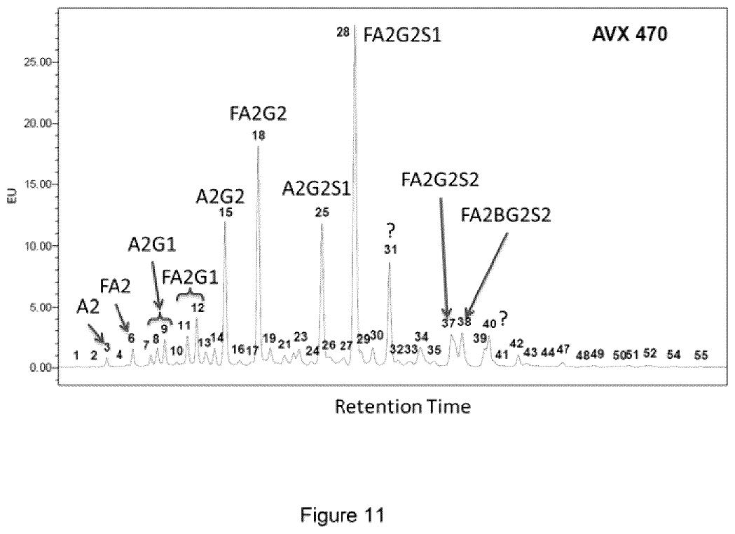

As discussed above, one factor that could confer protease resistance to colostral antibodies is glycosylation. To this end, the glycosylation pattern between AVX-470 and human IgG, and between bovine IgG from colostrum and serum were compared by hydrophilic interaction chromatography (HILIC)-ultra-high pressure liquid chromatography (UPLC) analysis. As shown in FIG. 10, AVX-470 and human IgG showed different patterns of glycosylation, with bovine colostral IgG being heavily sialylated. The identities of peaks for the most abundant species were confirmed by liquid chromatography/mass spectrometry (LC/MS) (Table 11 and FIG. 11).

The UPLC column was able to resolve 55 discrete peaks in the released sample. The retention times for each species were compared against those observed for a calibration standard consisting of glucose homopolymers to convert the peak retention times to Glucose Units (GU). The GU values are then compared to a reference database of known oligosaccharide species to identify the likely species in each peak. As shown in Table 11, most of the peaks with >10% abundance (highlighted in bold) in the chromatogram can be assigned by this method.

TABLE 11

| Peak | | Human IgG | AVX-470 | AVX-470 |
|---|---|---|---|---|
| ID | Structure | Average GU | Average GU | % Area |
| 1 | ND^{a} | NF^{b} | 4.96 | 0.04 |
| 2 | ND^{a} | NF^{b} | 5.25 | 0.03 |
| 3 | A2 | 5.44 | 5.45 | 0.49 |
| 4 | ND^{a} | NF^{b} | 5.73 | 0.08 |
| 5 | A2B | 5.81 | 5.82 | 0.13 |
| 6 | FA2 | 5.9 | 5.92 | 1 |
| 7 | Man5 | 6.2 | 6.21 | 0.77 |
| 8 | FA2B | 6.27 | 6.31 | 1.24 |
| 8 | A2[6]G1 | 6.27 | 6.31 | 1.24 |
| 9 | A2[3]G1 | 6.41 | 6.42 | 1.65 |
| 10 | A2B[6]G1 | 6.59 | 6.58 | 0.37 |
| 11 | A2B[3]G1 | 6.73 | 6.73 | 1.24 |
| 11 | FA2[6]G1 | 6.73 | 6.73 | 1.24 |
| 12 | FA2[3]G1 | 6.85 | 6.85 | 2.78 |
| 13 | FA2B[6]G1 | 6.97 | 6.97 | 1.14 |
| 14 | FA2B[3]G1 | 7.1 | 7.09 | 1.13 |
| 14 | Man6 | 7.1 | 7.09 | 1.13 |
| 15 | A2G2 | 7.23 | 7.22 | 8.33 |
| 16 | A2BG2 | 7.41 | 7.41 | 0.67 |
| 17 | ND^{a} | NF^{b} | 7.56 | 0.39 |
| 18 | FA2G2 | 7.66 | 7.65 | 12.96 |
| 19 | FA2BG2 | 7.81 | 7.8 | 1.54 |
| 20 | ND^{a} | NF^{b} | 7.9 | 0.21 |
| 21 | A2G1S(6)1 | 8 | 7.99 | 0.99 |
| 22 | FA2G1S(6)1 | 8.12 | 8.1 | 1 |
| 23 | ND^{a} | NF^{b} | 8.17 | 1.49 |
| 24 | ND^{a} | NF^{b} | 8.33 | 0.43 |
| 25 | A2G2S(6)2 | 8.48 | 8.46 | 9.47 |
| 26 | ND^{a} | NF^{b} | 8.54 | 1.2 |
| 27 | A2BG2S(6)2 | 8.74 | 8.73 | 1.11 |
| 28 | FA2G2S(6)1 | 8.9 | 8.88 | 22.23 |
| 29 | ND^{a} | NF^{b} | 8.97 | 0.48 |
| 30 | FA2BG2S(6)1 | 9.13 | 9.11 | 1.67 |
| 31 | ND^{a} | NF^{b} | 9.32 | 6.87 |
| 32 | ND^{a} | NF^{b} | 9.43 | 0.57 |
| 33 | Man9 | 9.62 | 9.59 | 0.61 |
| 34 | A2G2S(6)2 | 9.74 | 9.72 | 2.35 |
| 35 | A2BG2S(6)2 | 9.91 | 9.9 | 0.51 |
| 36 | NDª | NF^{b} | 9.97 | 0.03 |
| 37 | FA2G2S(6)2 | 10.15 | 10.13 | 3.6 |
| 38 | FA2BG2S(6)2 | 10.29 | 10.27 | 2.58 |
| 39 | ?S^{c} | 10.39 | 10.4 | 0.03 |
| 40 | ND^{a} | NF^{b} | 10.57 | 1.03 |
| 41 | ND^{a} | NF^{b} | 10.63 | 2.5 |
| 42 | ND^{a} | NF^{b} | 11.04 | 0.76 |
| 43 | ND^{a} | NF^{b} | 11.16 | 0.35 |
| 44 | ND^{a} | NF^{b} | 11.29 | 0.05 |
| 45 | ND^{a} | NF^{b} | 11.38 | 0.02 |
| 46 | ND^{a} | NF^{b} | 11.49 | 0.02 |
| 47 | ND^{a} | NF^{b} | 11.67 | 0.32 |
| 48 | ND^{a} | NF^{b} | 12 | 0.02 |
| 49 | ND^{a} | NF^{b} | 12.13 | 0.1 |
| 50 | ND^{a} | NF^{b} | 12.49 | 0.08 |
| 51 | ND^{a} | NF^{b} | 12.66 | 0.13 |
| 52 | ND^{a} | NF^{b} | 12.92 | 0.09 |
| 53 | ND^{a} | NF^{b} | 13 | 0.12 |
| 54 | ND^{a} | NF^{b} | 13.36 | 0.09 |
| 55 | ND^{a} | NF^{b} | 13.83 | 0.04 |

^{a}Not Determined
^{b}Not Found
^{c}GU value consistent with multiple possible sialylated species

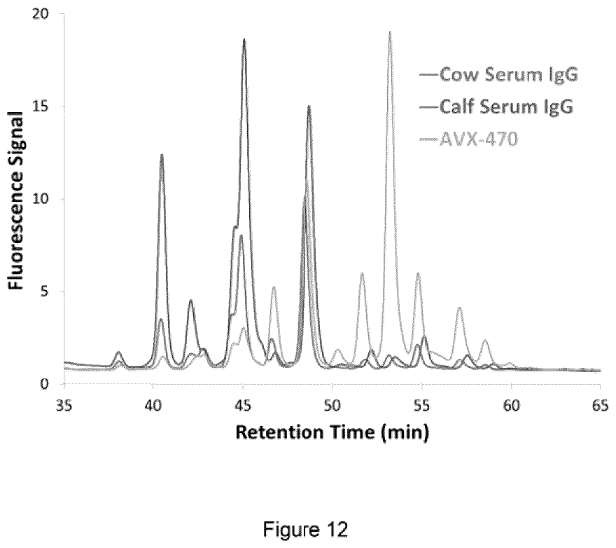

HILIC analysis was also used to assess differences in glycosylation patterns between bovine IgG derived from serum, colostrum, and milk. IgG was prepared by Protein G affinity chromatography, and the associated N-glycans from each sample were released with PNGase F, labeled with 2-AB, and separated on a GlycoSep N column. As shown in FIG. 12, the N-glycoprofile of IgG purified from the serum of a calf (which has never been pregnant) has slightly lower levels of the terminally galactosylated species G1F (rt=45 min) and G2F (rt=48 min), than IgG from the serum of a pregnant cow. Both serum IgGs have substantially less of the later-eluting species (likely mono- and disialylated oligosaccharides) than the colostral IgG sample (AVX-470).

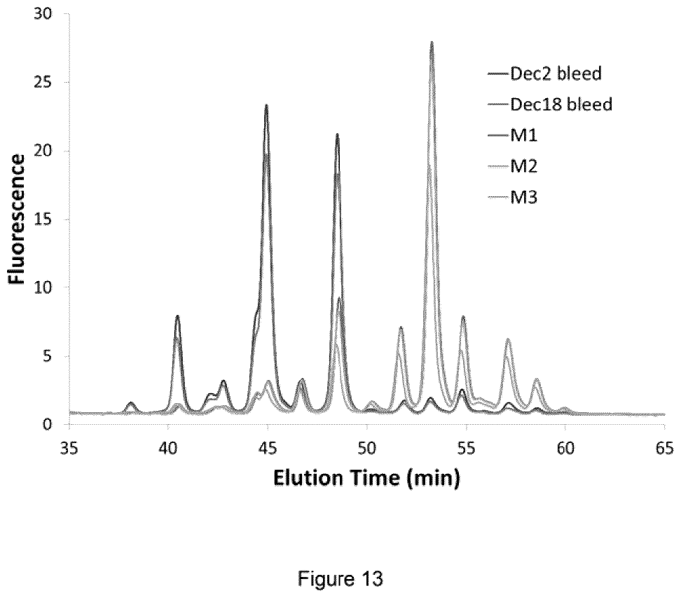

Changes in IgG N-glycosylation over time were also assessed by HILIC on samples taken from a single cow (cow #6003 from the ACICC-2 immunization study). Two serum samples were analyzed: one sample collected on Dec. 2, 2013 (peak titer for anti-TNF binding activity) and another collected on Dec. 18, 2013 (parturition date). IgG purified from colostrum collected in the first three milkings from this cow (collected over 1.5 days post-parturition) was also analyzed. As shown in FIG. 13, the N-glycosylation patterns for the serum samples are nearly identical to each other. Likewise, the three colostral IgG samples have very similar profiles. However, the N-glycosylation patterns of serum and colostral IgG populations are clearly distinct, with a substantial increase in the abundance of the later eluting species in the colostral IgG samples. Integration of the major peaks in each chromatogram (Table 12) indicates that the primary difference between serum and colostral samples is a substantial increase in the abundance of sialic acid-containing oligosaccharides in colostral IgG. In addition, there are small changes in core fucosylation and terminal galactosylation between the serum and colostral samples.

TABLE 12
Peak integration results for serum and colostrum samples

| Tenative | | | Serum Samples | | | Colostrum samples | | | |
|---|---|---|---|---|---|---|---|---|---|
| Peak # | assignment | RT(min) | Calf IgG | 6003Dec2 | 6003Dec18 | 6003M1 | 6003M2 | 6003M3 | AVX-470 |
| 1 | A2 | 38.1 | 1.1% | 1.0% | 1.0% | 0.1% | 0.2% | 0.2% | 0.3% |
| 2 | FA2 | 40.5 | 16.8% | 9.6% | 9.3% | 0.7% | 1.1% | 1.4% | 1.2% |
| 3 | A2G1 | 42.1 | 7.3% | 5.7% | 5.8% | 1.4% | 1.6% | 1.6% | 2.6% |
| 4 | FA2G1 | 45.1 | 41.4% | 40.2% | 41.5% | 5.9% | 6.2% | 6.7% | 6.7% |
| 5 | A2G2 | 46.8 | 1.3% | 3.4% | 3.3% | 3.7% | 3.7% | 3.5% | 7.6% |
| 6 | FA2G2 | 48.7 | 24.2% | 29.8% | 31.9% | 13.2% | 12.1% | 11.2% | 18.3% |
| 7 | A2G2S(6)1 | 52.2 | 1.5% | 1.5% | 1.5% | 9.6% | 9.6% | 9.6% | 9.4% |
| 8 | FA2G2S(6)1 | 53.5 | 1.4% | 2.0% | 1.9% | 40.8% | 40.5% | 40.2% | 32.2% |
| 9 | Unknown | 55.1 | 3.1% | 2.6% | 2.5% | 10.5% | 10.3% | 10.0% | 11.4% |
| 10 | A2G2S(6)2 | 57.5 | 1.4% | 1.4% | 0.9% | 9.7% | 10.3% | 10.9% | 7.1% |
| 11 | FA2G2S(6)2 | 59.0 | 0.6% | 2.8% | 0.5% | 4.4% | 4.6% | 4.8% | 3.2% |

| | | | | | | | |
|---|---|---|---|---|---|---|---|
| % fucosylation^{a} | 87.1% | 86.6% | 87.2% | 72.6% | 71.8% | 71.4% | 69.5% |
| % galactosylation^{b} | 82.1% | 89.4% | 89.7% | 99.1% | 98.8% | 98.5% | 98.4% |
| % sialylation^{c} | 8.0% | 10.3% | 7.3% | 74.9% | 75.3% | 75.4% | 63.3% |

^{a}Calculated as (sum of fucosylated species)/(sum of all identified peaks).
^{b}Calculated as (sum of galactosylated species)/(sum of all peaks).
^{c}Calculated as (sum of sialylated species)/(sum of all peaks)

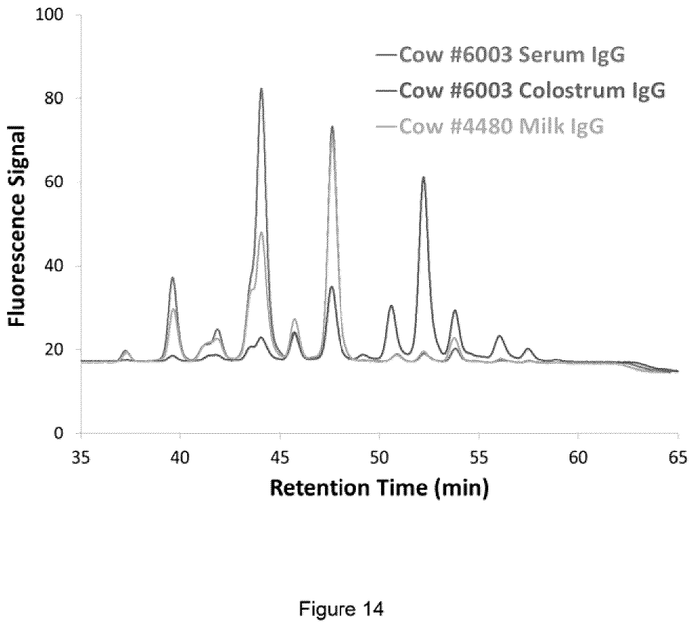

The transition from colostrum to milk is known to correspond with multiple changes in composition, including a substantial decrease in total immunoglobulin concentration and changes in N-glycosylation. As shown in FIG. 14, HILIC analysis of serum, colostrum and milk IgG samples demonstrates that colostral IgG glycosylation is the most distinct from the other fluids. Serum and milk IgG have similarly low levels of the later-eluting species which were tentatively identified as sialylated oligosaccharides.

The findings above collectively suggest that bovine colostral antibodies have a unique carbohydrate profile as compared with serum and milk antibodies.

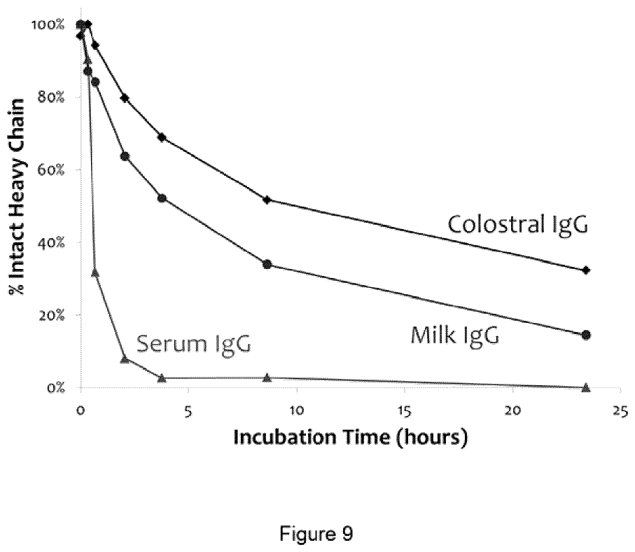

To determine whether this unique carbohydrate profile correlated with pancreatin stability, densitometry was used to quantify the IgG heavy chain band for digestion of serum, colostral, and milk IgG. As shown in FIG. 9, colostral antibodies were more resistant to pancreatin than serum and milk IgG.

### Example 5—Glycosylation does not Confer Protease Resistance to Colostral Antibodies

The previous Example demonstrated differences in glycosylation pattern between bovine serum antibodies and colostral antibodies, with colostral antibodies having heavily sialylated N-linked oligosaccharides linked to the Fc domain of colostral antibodies. The present experiment was conducted to determine whether this difference is responsible for conferring the unique protease resistance characteristic of colostral antibodies.

Pancreatin digestions of colostral IgG were carried out as described in Example 2, except that the pancreatin:substrate ratio was 2.5:1. One set of samples was also treated with neuraminidase (sialidase A). N-glycan analysis of this sample confirmed the complete removal of terminal sialic acid groups.

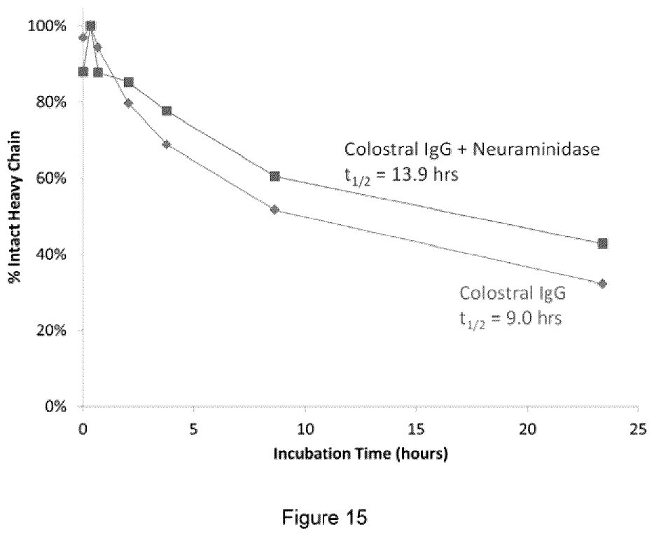

As shown in FIG. 15, the enhanced stability of the colostral IgG is independent of the presence of terminal sialic acid groups on the N-linked oligosaccharides, as neuraminidase treatment further stabilized the antibody (t1/2=13.9 hrs). This result suggests that sialylation is not responsible for the increase in proteolytic stability of colostral antibodies.

### Example 6—Stabilizing Entity in Colostrum is not Responsible for Protease Resistance of Colostral Antibodies

The hypothesis that a stabilizing entity present in the colostrum confers protease resistance to colostral antibodies was next considered.

A number of observations suggest against this possibility. First, purified colostral IgG antibodies, as shown in Example 3, are resistant to pancreatin digestion, suggesting that pancreatin resistance is likely independent of potentially stabilizing entities found in the colostrum. Second, when antibodies were purified from serum and colostrum collected from a single animal on the same day, SDS-PAGE did not detect any unique protein species in the colostral sample, i.e., there were no associated molecules (see FIG. 6). Finally, no evidence was found for a stabilizing entity in purified colostral IgG preparations by mass spectrometry that would confer protease resistance.

These findings suggest that the resistance of colostral antibodies to protease digestion is unlikely to be conferred by a stabilizing entity.

### Example 7—Purification of IgG1 and IgG2 from Colostrum and Serum

Previous studies have reported inconsistent and conflicting results regarding patterns of resistance to certain proteases of different bovine antibody isotypes. This led to the two hypotheses discussed above, i.e., the contribution of glycosylation or a stabilizing factor that conferred protease resistance to colostral antibodies.

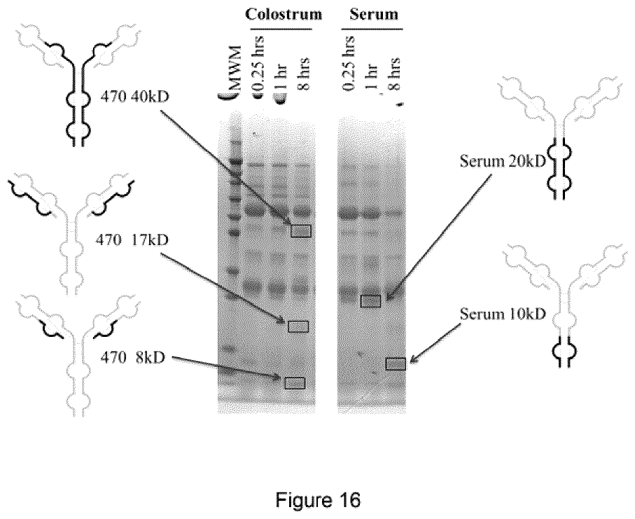

To better understand the differences in the pancreatin digestion pathway for bovine serum and colostrum antibodies, IgG from colostrum and serum were analyzed by reducing SDS-PAGE (FIG. 16). Several bands were identified as unique at later time points of either colostral or serum IgG digestion reactions. The 1 hour time point for degradation of serum IgG had a prominent band at 20 kDa, and the 8 hour time point had a strong band at 10 kDa. These bands were excised from the gel, digested in-gel with trypsin under denaturing conditions, then subjected to LC/MS/MS analysis to identify the proteins in each band (Table 13).

TABLE 13

| Sample | MW (kDa)^{a} | Top Protein Hit | Peptides Identified |
|---|---|---|---|
| AVX-470 | 8 | gi513137422^{b} | SPPSVTLFPPSTEELNGNK |
| | | | YAASSYLSLTSSDWK |
| AVX-470 | 17 | gi513137422^{b} | VSITCSGSSSNVGNGYVSWYQLIPGSAPR |
| | | | SPPSVTLFPPSTEELNGNK |
| | | | YAASSYLSLTSSDWK |
| | | | GSYSCEVTHEGSTVTK |
| AVX-470 | 40 | gi108750^{c} | VYPLSSCCGDK |
| | | | SSSTVTLGCLVSSYMPEPVTVTWNSGALK |
| | | | SGVHTFPAVLQSSGLYSLSSMVTVPGSTSGQTFTQNVAHPASSTK |
| | | | AVDPTCKPSPQDQPPPELPGGPSVFIFPPKPK |
| | | | FSWFVDDVEVNTATTKPR |
| | | | VHNEGLPAPIVR |
| | | | EPQVYVLAPPQEELSK |
| | | | STVSLTCMYTSFYPDYIAVEWQR |
| | | | NGQPESEDKYGTTPPQLDADSSYFLYSK |
| Senas IgC | 10 | gi896119^{d} | EPQVYVLDPPKEELSK |
| | | | TTPPQLDADR |
| Serum IgG | 20 | gi896119^{d} | EPSVFIFPPKPK |
| | | | VVSALPIQHQDWTGGK |
| | | | EPQVYVLQPPKEELSK |
| | | | TIPPQLDADR |
| | | | GDTYTCVVMHEALHNHYMQK |

^{a}Apparent molecular weight based on SDS-PAGE migration
^{b}Chain L., crystal structure of bovine antibody Blvlh12 with ultralong CDRH3
^{c}Ig heavy chain precursor (B/MT.4A.17.H5.AS) - bovine
^{d}Ig ganima-2 chain C region (clone 32.2) - bovine (fragment)

Based on the peptides identified in Table 13, along with the apparent molecular weight of each fragment observed by reducing SDS-PAGE, each band was assigned to a fragment of the original antibody. The 8 kDa and 17 kDa bands observed in the 8 hour time point map to the light chain, while the 40 kDa band is most likely the constant domains from the heavy chain. These results suggest that the initial cleavage events occur near the N- and C-termini of the heavy and light chains, while leaving the core structure around the hinge intact. In contrast, the 20 kDa bands from the early (1 hour) time point in the serum IgG digest map to the Fc domain, consistent with cleavage in the hinge. The 10 kDa band that appears later in the serum IgG digestion reaction maps only to peptides in the CH3 domain, suggesting that the Fc is further degraded over time. These results indicate that the hinge region of colostral IgG is protected from proteolytic cleavage relative to the serum IgG antibody. In addition, the colostral IgG sequences map predominantly to the IgG1 subtype, while the serum IgG sample maps to the IgG2 sequence.

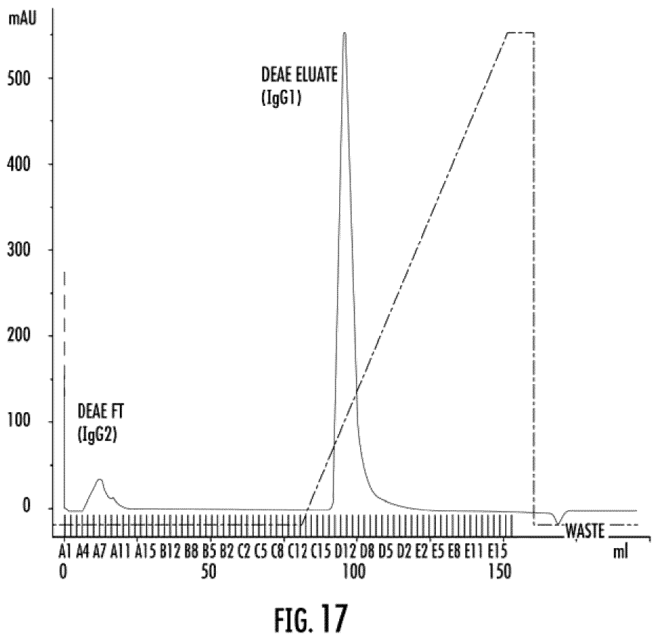

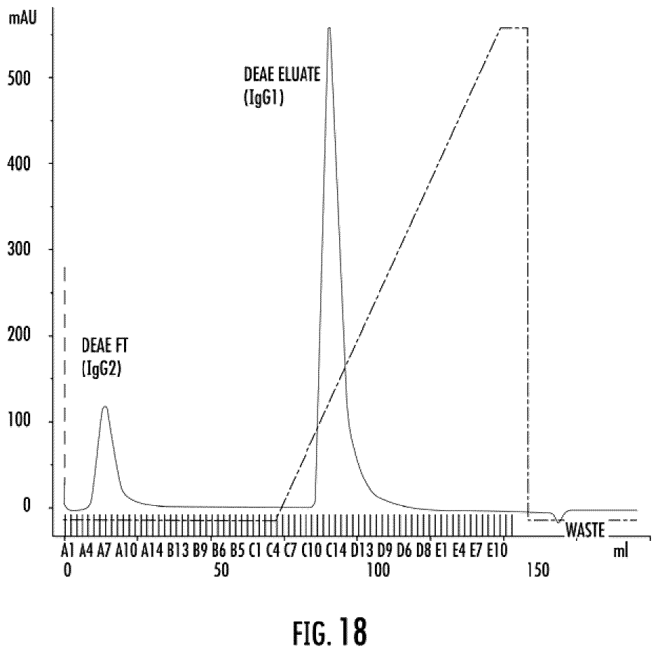

To specifically investigate the impact of IgG subtype on protease susceptibility, IgG was prepared from colostrum and serum by Protein G affinity chromatography, and separated into IgG1- and IgG2-enriched fractions with a DEAE Sepharose column (FIGS. 17 and 18, respectively). As a sufficient amount of IgG1 could not be purified from the serum of pregnant cows, calf serum was used as the source of serum IgG1.

As shown in Table 14, the DEAE flow-through fraction was pure IgG2, as determined by a sandwich ELISA method with subtype-specific antibodies. The fraction eluted with the salt gradient was enriched in IgG1, but often contained trace amounts of IgG2. Others have also observed this phenomenon, and linked it to the presence of variable domains that have unusually low isoelectric points (Butler et al., 1987). To remove this contamination, the DEAE eluates were passed over a column containing immobilized anti-IgG2, which removed the contaminating IgG2 and yielded a pure IgG1 fraction (Table 14, 6003M1 DEAE+anti-IgG2 and calf #24 DEAE+anti-IgG2).

TABLE 14
Isotype ELISA results for DEAE fractions.

| | ng IgG1 per | ng IgG2 per | % |
|---|---|---|---|
| Sample | μg total IgG | μg total IgG | IgG1 |
| AVX-470 DEAE + anti-IgG2 | 668 | 0 | 100% |
| 6003M1 IgG | 772 | 122 | 86% |
| 6003M1 DEAE FT | 0 | 1253 | 0% |
| 6003M1 DEAE eluate | 947 | 24 | 98% |
| 6003M1 DEAE + anti-IgG2 | 845 | 0 | 100% |
| Calf #24 serum IgG | 811 | 290 | 74% |
| Calf #24 DEAE FT | 0 | 1910 | 0% |
| Calf #24 DEAE eluate | 1083 | 0 | 100% |
| Calf #124 DEAE + anti-IgG2 | 1043 | 0 | 100% |

### Example 8—Pancreatin Digestion of Bovine IgG1 and IgG2

In this experiment, bovine IgG1 and IgG2 purified from serum (Calf #24) or colostrum (6003M1=early colostrum from Cow #6003; AVX-470=colostrum pooled from 20 cows) were digested with pancreatin and subjected to reducing SDS-PAGE as described in Example 3.

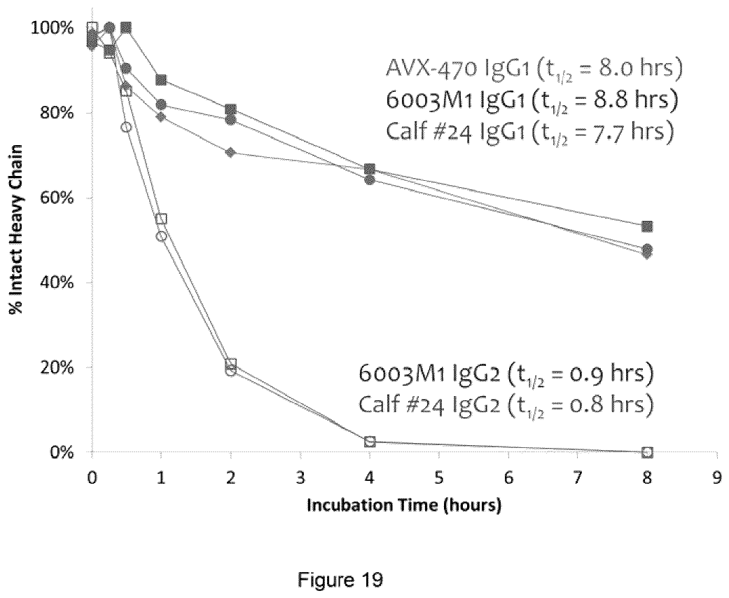

As shown in FIG. 19, IgG1 fractions were relatively stable against pancreatin digestion, with half-lives ranging from 7.7 to 8.8 hours. In contrast, IgG2 fractions from both serum and colostrum were rapidly degraded, with half-lives of 0.8-0.9 hours. Thus, IgG1 showed a 10-fold increase in half-life relative to IgG2. This data suggests that IgG isotype, specifically the IgG1 isotype, is the key determinant that confers protease resistance of bovine colostral antibodies.

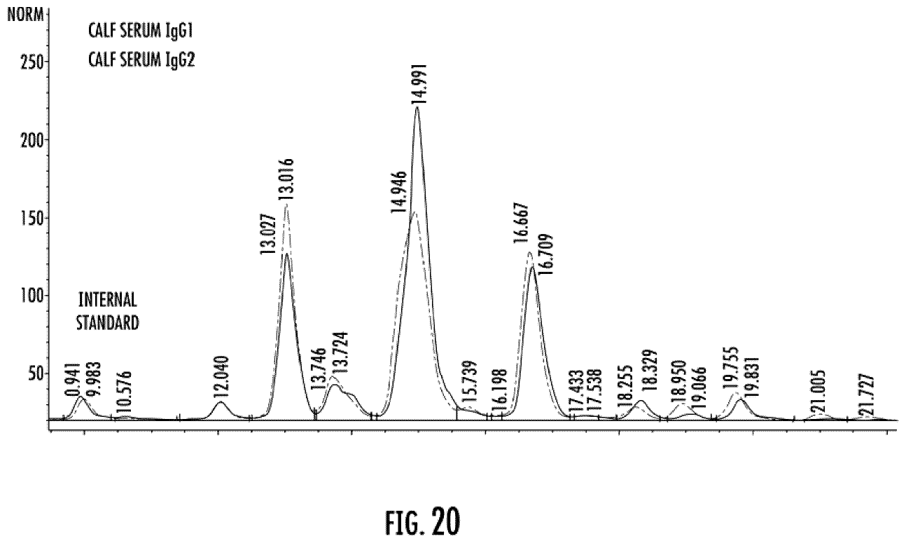

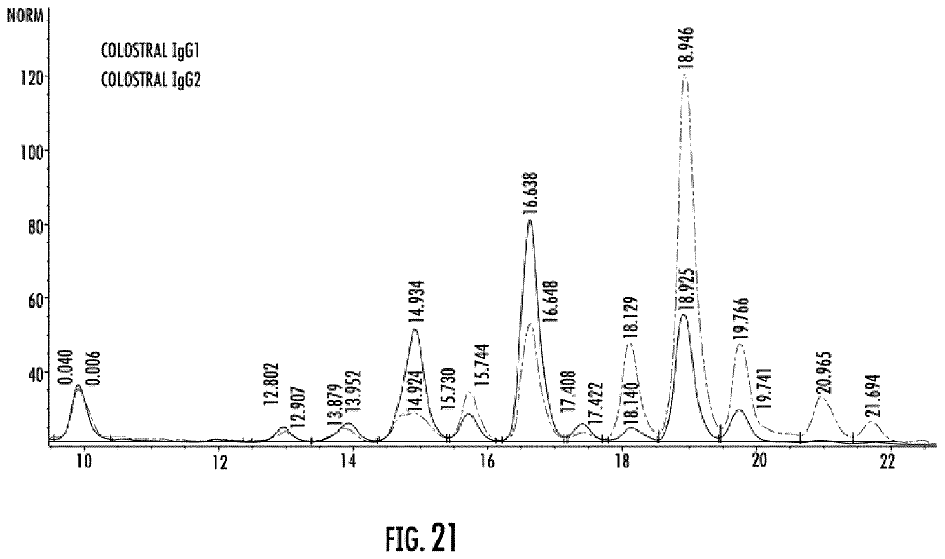

Carbohydrates on bovine isotypes from serum were also compared between IgG1 and IgG2. N-linked glycans were released enzymatically, fluorescently labeled with 2-aminobenzamidine and separated by hydrophilic interaction chromatography (HILIC) for quantitation. The oligosaccharide species present in each HILIC peak was determined by a combination of mass spectrometry and retention-time comparison with known standards. As shown in FIG. 20, there were no significant differences in fucosylation, galactosylation, and sialylation between bovine IgG1 and bovine IgG2 from serum. When comparing bovine IgG1 and bovine IgG2 from colostrum, both isotypes were sialylated, with higher sialylation and less fucosylation found in IgG1 (FIG. 21).

This result, in combination with results from the preceding Examples, suggests that the source of the antibody (i.e., colostrum or serum) determines the glycosylation pattern, while IgG isotype determines protease resistance.

### Example 9—Differences in Amino Acid Sequences Between Bovine IgG1, Bovine IgG2, and Human IgG1, and Comparisons with Other Ruminant IgG1 Sequences

To gain further insight into factors that contribute to the stability of bovine IgG1 antibodies, the primary structures of bovine IgG1, bovine IgG2, and human IgG1 were compared.

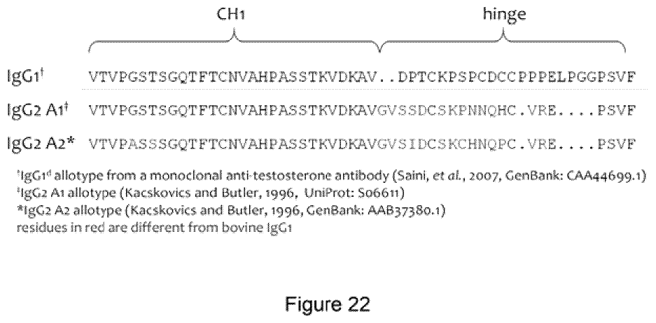

FIG. 22 shows a comparison of the CH1-hinge region sequences from bovine IgG1 and bovine IgG2. Bovine IgG1 has four cysteine residues in the hinge region, whereas the A1 and A2 allotypes of bovine IgG2 have two or three cysteine residues, respectively. Moreover, bovine IgG1 has a higher incidence of proline residues than the IgG2 sequences. Proline residues are known to inhibit cleavage by many endoproteinases, and can induce a more rigid structure to the hinge. The IgG2 upper hinge also has a two-residue insertion “GV”, which may introduce additional structural flexibility.

FIG. 23 shows a comparison of the CH1-hinge region of human IgG1 and bovine IgG1. Several of the sequence differences between human IgG1 and bovine IgG1 occur at sites known to be labile towards degradation by human proteases (Brerski et al., mAbs 2011; 3:558-67) or spontaneous peptide bond cleavage at acidic pH (Cordoba et al., J Chromatography B 2005; 818:115-21). The absence of these sites in the bovine IgG1 hinge may also contribute to the proteolytic stability of bovine IgG1.

The hinge sequences of other ruminant species were searched for homology to the following peptide:

Bovine IgG1 (AAB37381.2):
(SEQ ID NO: 91)
DKAVDPRCKTTCDCCPPPELPGGP

This sequence was used as a query in a BLAST search against the entire non-redundant protein sequence database. The top hits from this search were as follows:

TABLE 15

| SEQ | | |
|---|---|---|
| ID | Species | Sequence |
| 5 | Bos taurus (cow) IgG1a | VDK--AVDP---RCK-TTCD-C-CPPPELPGGPSVF |
| 6 | Bos taurus (cow) IgG1c | VDK--AVDP---RCK-RPCD-C-CPPPELPGGPSVF |
| 7 | Bos taurus (cow) IgG1b,d | VDK--AVDP---TCKPSPCD-C-CPPPELEGGPSVF |
| 8 | Ovis aries (sheep) | VDK--RVEP---GCP-DPCKHCRCPPPELPGGPSVF |
| 8 | Ovis aries (sheep) | VDK--RVEP---GCP-DPCKHCRCPPPELPGGPSVF |
| 8 | Ovis aries (sheep) | VDK--RVEP---GCP-DPCKHCRCPPPELPGGPSVF |
| 9 | Lama glama (llama) | VDK--RVEPHG-GCT---CP--QCPAPELPGGPSVF |
| 9 | Vicugna pacos (alpaca) | VDK--RVEPHG-GCT---CP--QCPAPELPGGPSVF |
| 9 | Camelus dromedarius (Arabian camel) | VDK--RVEPHG-GCT---CP--QCPAPELPGGPSVF |
| 10 | Meriones unguiculatus (gerbil) | VDK--TVEPRGTKHICPDCP--KCPAPDLSGGPSVF |
| 11 | Felis catus (cat) | VDKTVRKTDHP-PGP-KPCDCPKCPPPEMLGGPSIF |
| 12 | Oryctolagus cuniculus (rabbit) | VDK--TVAP--STCSKPT-----CPPPELLGGPSVF |

The three Bos taurus sequences reflect individual allotypes. The most closely related sequence is the sheep IgG1, which retains the key features of the bovine hinge (three inter-HC disulfide bonds, deletion of the known proteolysis sites). The dromedary IgG1 sequences (llama, alpaca and camel) are also very similar. The next closest hits are from gerbil and cat IgG1, which do not align as well. The cat sequence has only two inter-heavy-chain disulfide bonds. The gerbil sequence contains three cysteines, but it is unclear if the most N-terminal cysteine forms an inter-HC disulfide bond, or a HC-LC linkage. The rabbit IgG sequence contains the disulfide link between the N-terminus of the CH1 domain and the upper hinge, but only a single inter-HC disulfide bond and a significantly shorter hinge than is observed for IgG1 from other species.

### Example 10—Expression and Pancreatin Stability of Bovine IgG1 Anti-Testosterone Monoclonal Antibody

A bovine IgG1 anti-testosterone antibody was generated based on the anti-testosterone antibody produced by the heterohybridoma described in Jackson et al. (Molecular Immunology 1992; 29:667-76). The heavy and light chain amino acid sequences are as follows:

Bovine anti-testosterone IgG1 antibody (light
chain):
(SEQ ID NO: 23)
QAVLGQPSSVSGSLGQRVSITCSGSSSNIGTYGVEWYQQVPGSGLRTII
YGSNSRPSGVPDRFSGSKSGNTATLTISSLQAEDEADYFCAAGDSSSRG
AVFGSGTLTALGQPKSPPSVTLFPPSTEELNGNKATLVCLISDFYPGSV
TVVWKADGSTITRNVETTRASKQSNSKYAASSYLSLTSSDWKSKGSYSC
EVTHEGSTVTKTVKPSECS
Bovine anti-testosterone IgG1 antibody (heavy
chain):
(SEQ ID NO: 24)
QVQLRESGPSLVKPSQTLSLTCTVSGFSLSSYALTWVRQAPGKALEWVG
GITSGGTTYYNPALKSRLSITKENSKSQVSLSVSSVTPEDTATYYCARS
TYGEVGDGAIADAWGQGLLVTVSSASTTAPKVYPLSSCCGDKSSSTVTL
GCLVSSYMPEPVTVTWNSGALKSGVHTFPAVLQSSGLYSLSSMVTVPGS
TSGQTFTCNVAHPASSTKVDKAVDPTCKPSPCDCCPPPELPGGPSVFIF
PPKPKDTLTISGTPEVTCVVVDVGHDDPEVKFSWFVDDVEVNTATTKPR
EEQFNSTYRVVSALRIQHQDWTGGKEFKCKVHNEGLPAPIVRTISRTKG
PAREPQVYVLAPPQEELSKSTVSLTCMVTSFYPDYIAVEWQRNGQPESE
DKYGTTPPQLDADSSYFLYSKLRVDRNSWQEGDTYTCVVMHEALHNHYT
QKSTSKSAGK

DNA constructs encoding the heavy and light chains above were produced and expressed, and the resulting antibodies (50 μg/mL) were subjected to pancreatin digestion (1 mg/mL) and tested for anti-testosterone activity by ELISA. Pancreatin digestion was performed as described in Example 3.

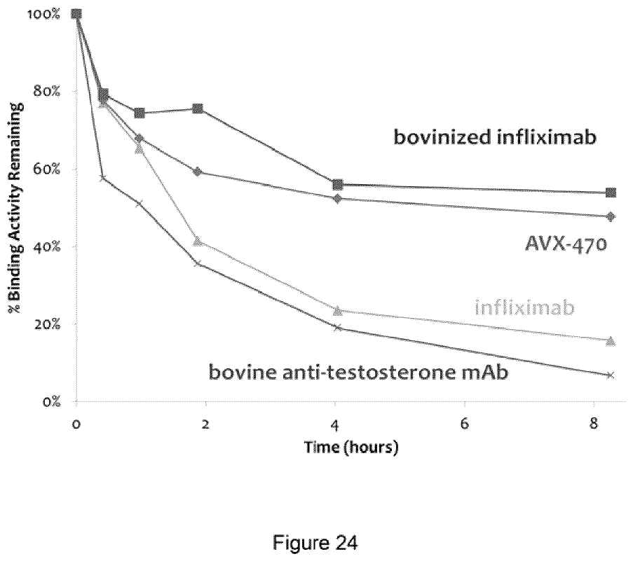

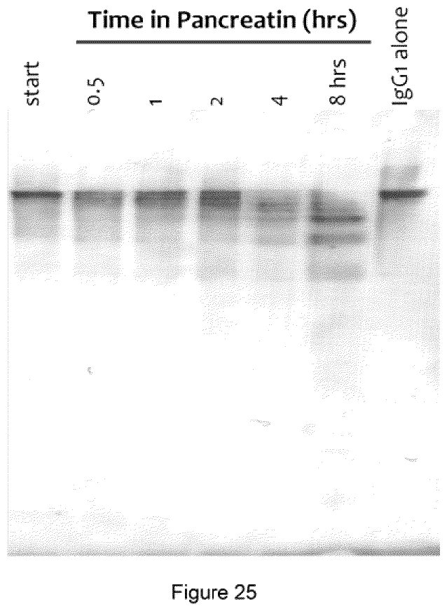

As shown in FIG. 24, pancreatin-digested anti-testosterone antibody lost binding activity to testosterone at a much faster rate than AVX-470 and bovinized infliximab. This result appears to be inconsistent with bovine IgG1 being stable to digestion by intestinal proteases. However, when digested anti-testosterone antibody samples were subjected to SDS-PAGE (probed with anti-bovine IgG1) as shown in FIG. 25, multiple bands appear at slightly lower molecular weights, suggesting that small pieces of the antibody are being clipped off by the proteases, possibly in the antigen-binding region. This suggests that potential protease cut sites in the antigen-binding region is also a consideration when generating a stable bovine IgG1 antibody.

### Example 11—Immunization of Cows with TNF and Adjuvants

The current immunization method used in the production of AVX-470 drug product for the Phase 1b clinical trial consists of a series of three subcutaneous injections of recombinant human TNF plus Quil A adjuvant at 2-3 week intervals in pregnant Holstein dairy cows prior to calving. These conditions were selected based on the outcome of a short series of studies conducted in calves that evaluated multiple adjuvants, antigen levels, and dosing frequency, as discussed below.

### Calf Study #1

In the first study, a series of adjuvants were evaluated in male Holstein calves, aged 3-5 months, immunized with adjuvinized rhTNF (Cell Sciences, Canton, MA) by subcutaneous injection. Four different adjuvants were evaluated—Emulsigen-D, Carbigen, Quil A, and Seppic ISA, in combination with 0.05 mg rhTNF. Serum samples were collected prior to each immunization and 14-21 days after the final immunization.

TABLE 16

| | | Serum | Serum collection - |
|---|---|---|---|
| | | collection - | Day post- |
| Sample name | Inoculation | Date | inoculation |
| Serum sample set #1 | Pre-bleed | Jan. 11, 2011 | (−) |
| Serum sample set #2 | 1 | Feb. 1, 2011 | 21 |
| Serum sample set #3 | 2 | Feb. 15, 2011 | 14 |
| Serum sample set #4 | 3 | Mar. 8, 2011 | 21 |
| Serum sample set #5 | 4 | Mar. 22, 2011 | 14 |

Serum was prepared from pre-immunization bleeds (“pre-bleed”) and after each of 4 inoculations from individual calves as shown in Table 16. Thus, 5 separate Calf Study 1 sample sets were generated for each adjuvant; however, only sample set 1 (pre-bleed), set 4 (after 3 inoculations), and set 5 (after 4 inoculations), were analyzed for rhTNF-binding activity by direct ELISA on rhTNF-coated plates and for rhTNF-neutralizing activity in the L929 cytotoxicity assay, which was carried out as described in US2012/0258118.

Individual calf sera and pools of sera for each group from set 4 (3 inoculations) and set 5 (4 inoculations) were analyzed for TNF-binding activity in anti-TNF ELISA assays at dilutions ranging from 1:10 to 1:10E6. TNF-binding titers were calculated as the inverse dilutions giving 50% of maximal binding (1/EC₅₀).

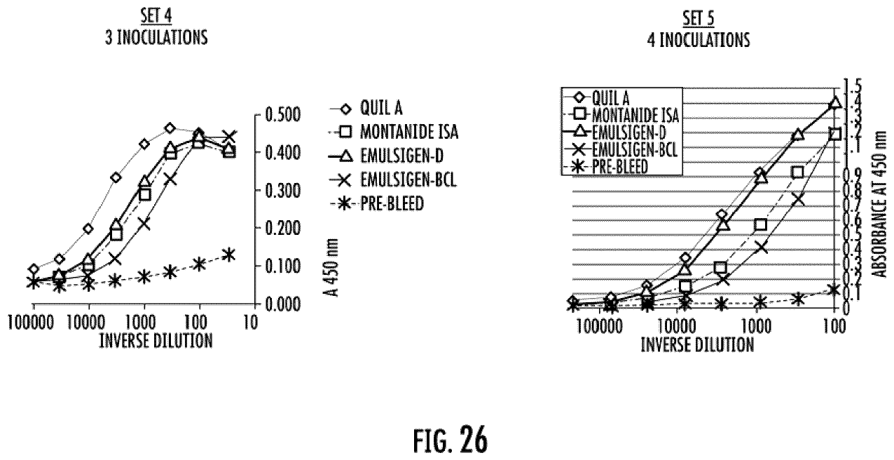

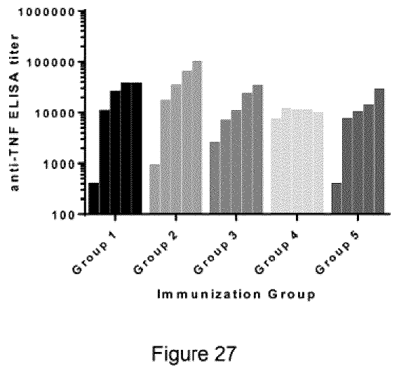

The data in FIGS. 26 and 27, and Tables 17 and 18 (shows data from two independent experiments), show that titers of pooled sera were highest in the Quil A group, followed by Emulsigen-D, Montanide and Emulsigen-BCL. Importantly, there was little or no increase in titer after the 4^{th} inoculation in the Quil A and Montanide groups, whereas there was an increase in the Emulsigen-D group and a decrease in the Emulsigen-BCL group.

TABLE 17

| | | | | | | | | | | Emulsigen | | |
|---|---|---|---|---|---|---|---|---|---|---|---|---|
| Adjuvant | Quil A | | | Montanide | | | Emulsigen-D | | | BCL | | |
| Calf # | 1 | 2 | 3 | 4 | 5 | 6 | 7 | 8 | 9 | 10 | 11 | 12 |
| Titer | 2000 | 4000 | 2000 | 1000 | 100 | 2000 | 1000 | 3000 | 5000 | 500 | 1000 | 600 |

Calf sera were then analyzed for TNF-neutralizing antibody titers in the L929 cytotoxicity assay. As shown in Table 18, Quil A showed the highest TNF-neutralizing antibody titers relative to the other adjuvant groups.

TABLE 18

| | 1^{st} | 2^{nd} |
|---|---|---|
| Calf Study 1 Sample | experiment | experiment |
| Quil A, Set 4 (3 | 53,000 | 42,000 |
| inoculations) | | |
| Quil A, Set 5 (4 | 175,000 | 175,000 |
| inoculations) | | |
| Montanide ISA-201, Set 4 | 43,000 | 40,000 |
| Emulsigen-D, Set 4 | 16,000 | 11,000 |
| Emulsigen-BCL, Set 4 | 9,100 | 7,100 |

Another key distinction from the ELISA data is the 3-4-fold increase in potency between set 4 (3 inoculations) and set 5 (4 inoculations) of the Quil A group, whereas the ELISA data showed that the extra boost did not significantly increase binding antibody titer (from 5000 to 5500; FIG. 26 and Table 17).

### Calf Study #2

In this study, 0.05 mg of rhTNF with Quil A was compared to a 10-fold higher antigen dose (0.5 mg per injection). The 0.5 mg rhTNF dose was tested with three different adjuvants—Quil A, Montanide ISA-25, and Montanide ISA-201. An additional group of calves received an even larger dose of rhTNF (1 mg) with Quil A. Three vaccinations at 3 week intervals were conducted, and an extended series of immunizations was also included, with 4^{th} and 5^{th} vaccinations.

Fifteen male Holstein (13) or Holstein-cross (2) dairy calves aged 3-5 months were used. Sera were generated by immunizing 3 calves per group 5 times by subcutaneous injection at 3 week intervals with different doses of recombinant human tumor necrosis factor (rhTNF; Cell Sciences, Canton, MA) ranging from 0.05 to 1.0 mg, plus one of three adjuvants: Quil A (Groups 1-3), Montanide ISA-201 (Group 4) or Montanide ISA-25 (Group 5) (Table 19).

TABLE 19

| Group | 1 | 2 | 3 | 4 | 5 |
|---|---|---|---|---|---|
| rhTNF antigen | 0.05 mg | 0.5 mg | 1 mg | 0.5 mg | 0.5 mg |
| Adjuvant | Quil A | Quil A | Quil A | Montanide | Montanide |
| | | | | ISA-201 | ISA-25 |

Serum was prepared from individual calves from pre-immunization bleeds (“pre-bleed”) and 3 weeks after each of 5 inoculations. Sera from individual calves and pooled sera from each group were analyzed for rhTNF-binding activity by direct ELISA on rhTNF-coated plates and for rhTNF-neutralizing activity in the L929 cytotoxicity assay as in Calf Study 1.

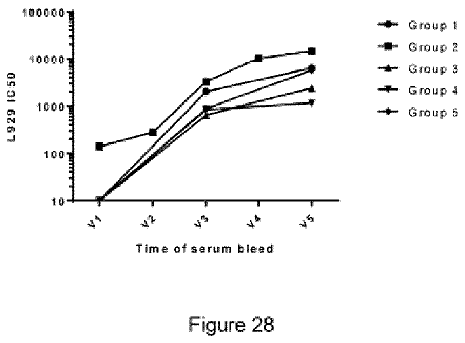

The immunization regimen used in Group 2, which consisted of 0.5 mg of rhTNF plus Quil A, was found to produce the highest peak titer of anti-TNF antibodies after each of the first three immunizations as measured in a TNF ELISA assay (FIG. 27), and the highest TNF neutralizing potency in the L929 cell-based assay (FIG. 28). A comparison among Groups 3, 4 and 5 showed that Quil A was the most effective adjuvant. The difference in both TNF binding titers and neutralization potency between Group 1 and 2 was considered insignificant after 3 vaccinations. Therefore the lower amount of antigen (0.05 mg rhTNF) was selected for immunization regimens involving three vaccinations.

### Single Immunization Regimen in Pregnant Cows

This study evaluated a single immunization regimen in pregnant cows based on results from Calf Studies 1 and 2, and evaluated whether the calf data could be extrapolated to pregnant cows.

Five pregnant Holstein cows (80-85 days pre-partum) were immunized four times subcutaneously over a 75 day period with recombinant human TNF (0.5 mg per immunization) mixed with Quil-A adjuvant.

As shown in Table 20, the peak anti-TNF ELISA titers (AU/mL) for each of the 5 cow colostrum samples was seen at milking 1. Similar results were observed with 20 cows immunized 3 times with 0.05 mg rhTNF. There were no significant differences in TNF-binding antibody titers between the 5-cow (4 inoculations with 0.5 mg rhTNF) or the 20-cow (3 inoculations with 0.05 mg rhTNF) studies.

TABLE 20

| | 5 cow | | 20 cow | |
|---|---|---|---|---|
| | Average | STD | Average | STD |
| Pool | 6,479 | 6,157 | 8,140 | 5,918 |
| Milking-1 | 44,686 | 45,356 | 60,335 | 37,816 |
| Milking-2 | 15,593 | 15,521 | 28,008 | 15,810 |
| Milking-3 | 4,499 | 4,017 | 9,424 | 6,480 |
| Milking-4 | 1,806 | 1,314 | 3,663 | 3,391 |
| Milking-5 | 843 | 611 | 1,862 | 2,451 |
| Milking-6 | 602 | 480 | 845 | 1,185 |
| Milking-7 | 558 | 450 | 542 | 710 |
| Milking-8 | 609 | 387 | 426 | 619 |

### Example 12—Immunization of Calves with Various Antigens

Five different antigens or antigen combinations were used to immunize male dairy calves to generate polyclonal bovine colostral antibodies for potential use as gut-targeted therapeutics. Certain antigens, such as IL-6Rα, IL-12/23p40 and MAdCAM-1, were selected based on known expression in gastrointestinal tissues, especially in patients with inflammatory bowel disease, and their promising potential as targets for antibody-based therapies (Veldman, 2006; Reenaers et al., 2012). The gluten/gliadin-derived peptide antigens PT-gliadin (van de Wal et al., 1998) and gliadin 33-mer (peptide 56-88; Shan et al., 2002) relate to the concept of generating gluten-neutralizing antibodies for reduction of trace gluten levels in the gut lumen in celiac disease patients. The gliadin 33-mer peptide has been shown to be the immunodominant peptide in the gluten/gliadin molecule in celiac disease patients (Shan et al., 2002; Matysiak-Budnik et al., 2003).

Based on the studies described in Example 11, Quil A adjuvant was used in the immunization studies described below.

### Antigen Preparation

Six distinct peptide/protein antigen solutions were prepared from commercially available sources.

A Peptic/Tryptic digest of gliadin (PT-Gliadin) was prepared in house using methodology adapted from van de Wal et al, 1998. Briefly, 1 gram of gliadin (Sigma, Cat #G3375) was dissolved in 10 mL 1M acetic acid for 3 days at room temperature, then boiled for 10 minutes and treated with 10 mg pepsin (Sigma Cat #P6887) for 4 hours at 37° C. The pH was adjusted to 7.8 with NaOH and then treated with 10 mg trypsin (Sigma Cat #T1426-50) for 4 hours at 37° C. Digestion was stopped by adding 10 mg trypsin inhibitor (Sigma Cat #T6522) and incubating at 2-4° C. overnight. Particulates were removed by centrifugation and the solution was dialyzed against phosphate buffered saline (PBS) pH 7.4 using a Spectra/Por Float-a-Lyzer G2 device with an 8-10 kDal cutoff. The final PT-gliadin preparation showed bands of 20 kDal and below by SDS-PAGE, whereas the dominant bands in undigested gliadin were 40-50 kDal. Peptide content was measured by bicinchoninic acid (BCA) assay calibrated with gliadin.

Gliadin 33-mer (H-Leu-Gln-Leu-Gln-Pro-Phe-Pro-Gln-Pro-Gln-Leu-Pro-Tyr-Pro-Gln-Pro-Gln-Leu-Pro-Tyr-Pro-Gln-Pro-Gln-Leu-Pro-Tyr-Pro-Gln-Pro-Gln-Pro-Phe-NH2; SEQ ID NO: 25) peptide was synthesized by Bachem (Torrance, CA, catalog #4077401) and represents residues 56-88 in the gliadin molecule (Shan et al., 2002).

Other protein antigens were: Recombinant Mouse IL-6Rα (carrier free) from R & D Systems (Cat #1830-SR/CF, lot #ITY141301A; Minneapolis, MN); Recombinant Mouse IL-6 from Cell Sciences (Cat #CRI130C, lot #3201610; Canton, MA); Carrier Free Recombinant Mouse 1L-12/1L-23 p40 Homodimer from R& D Systems (Cat #499-ML/CF, lot #RH041212A; Minneapolis, MN) and Recombinant Mouse MAdCAM-1 Fc Chimera from R & D Systems (Cat #993-MC-050, lot #EOH1312101; Minneapolis, MN).

### Animals/Immunization Groupings

Fifteen 9-11 month old neutered male Holstein dairy calves were used for the study, and they were separated into five groups with three calves each. The calf groups are summarized in Table 21.

TABLE 21

| | | | | | E |
|---|---|---|---|---|---|
| | A | | | | IL-6R, IL-12/IL-23 |
| | Peptic/tryptic | B | | | p40 and mucosal |
| | digest of | Gliadin | | | addressin cell adhesion |
| | gliadin | 33-mer | C | D | molecule-1 |
| Group | (PT-Gliadin); | peptide; | IL-6 receptor | IL-6 and IL-6R; | (MAdCAM-1); 0.05 mg |
| Antigen | 0.5 mg | 0.5 mg | (IL-6R); 0.05 mg | 0.05 mg each | each |
| Adjuvant | Quil A | Quil A | Quil A | Quil A | Quil A |
| Calf ID # | 26 | 22 | 21 | 29 | 23 |
| | 28 | 30 | 27 | 35 | 24 |
| | 33 | 32 | 34 | 36 | 31 |

All groups underwent the same immunization regimen. Immunizations took place on day 0 (V1), day 21 (V2), day 42 (V3), and day 63 (V4). Each dose was approximately 2 cc in volume and immunizations were delivered subcutaneously in the shoulder or neck area.

Blood samples were taken before each immunization and the last sample was taken 21 days after the last immunization. Approximately 40 milliliters of blood was collected via venipuncture at each time point. Blood was allowed to clot and then centrifuged if needed to separate serum per standard procedures. The serum was aliquoted into two duplicate samples, and stored at −20° C. A large volume serum collection (40-50 ml serum from ˜100 ml blood) was performed at the final collection date.

### ELISA and Analysis of Samples

To determine if calves produced antibodies to the antigen, serum samples were tested with direct ELISAs. ELISA conditions are shown in Table 22. Plates were washed with PBS-T (PBS pH 7.4 with 0.05% Tween-20) three times using a multi-channel matrix pipette. Wells in the plate were blocked by adding 300 uL per well of 2% Superblock solution (Thermo Fisher #37515) for 30 minutes to an hour at room temperature. Serum samples were initially diluted to a 1:200 initial dilution and tittered down 96 V-well dilution plates using serial three-fold dilutions for a seven-point curve. Reference curves were diluted to appropriate starting concentration depending on the ELISA and were tittered down the plate using serial three-fold dilutions for a seven-point curve. Dilutions were made in 2% Superblock in PBS-T. Blocking solution was removed from ELISA plates and plates were washed three times with PBS-T. Diluted Serum samples and reference curves were transferred to ELISA plates and incubated for one hour. Plates were washed three times and antibody was detected with the addition of 100 uL per well of horse radish peroxidase (HRP)-labeled secondary antibody to bovine Ig (sheep anti-bovine IgG (H+L)) for serum samples and an appropriate HRP labeled antibody for reference curves. Secondary antibody was incubated for one hour. After washing plates three times, 100 uL per well of TMB substrate was added to plates and allowed to develop for twenty minutes after which 100 uL of 1% Sulfuric Acid was added to wells. Plates were read using a Biotek Epoch (Winooski, VT) microplate reader at 450 nm. Background absorbance values from wells containing reagent buffer only were averaged and subtracted from all wells for final blanked A450 nm data. Data were analyzed using Gen5 software with a computer-generated 4-PL curve-fit for sample curves and reference curves. Antibody titer, defined as the dilution which resulted in a 0.2 OD (y=0.2), was calculated for each sample curve and the values were used to generate a curve for each calf serum and for the group pooled serum to represent progression of antibody concentration in serum through the different immunizations.

TABLE 22

| Study | ELISA | | | Reference | |
|---|---|---|---|---|---|
| Group | coating | Coating | Reference | Curve | Coating |
| Tested | Antigen | Concentration | Curve | Secondary | Condition |
| Group A | PT-Gliadin | 100 ug/mL | Rabbit anti- | HRP Goat anti- | One hour at |
| | | | gliadin (Sigma | rabbit (Bethyl | 37° C. |
| | | | #G9144) | #A120-101P) | |
| Group B | Gliadin 33-mer | 20 ug/mL | Rabbit anti- | HRP Goat anti- | Two hours |
| | | | gliadin (Sigma | rabbit (Bethyl | are 37° C. and |
| | | | #G9144) | #A120-101P) | overnight at 4° C. |
| Group A; | Gliadin in | 20 ug/mL | Rabbit anti- | HRP Goal anti- | One hour at |
| Group B | Acetic Acid | | gliadin (Sigma | rabbit (Bethyl | 37° C. |
| | | | #G9144) | #A120-101P) | |
| Group A; | Gliadin in | 20 ug/mL | Rabbit anti- | HRP Goat anti- | Two hours |
| Group B | Ures | | gliadin (Sigma | rabbit (Bethyl | are 37° C. and |
| | | | #G9144) | #A120-101F) | overnight at 4° C. |
| Group C; | IL-6Rα | 500 ng/mL | Goat anti- | HRP Goat anti- | Room |
| Group D; | | | mouse IL-6R | rabbit (Bethyl | temperature |
| Group E | | | (R&DSystems | #A120-191P) | for one hour |
| | | | #AF1830) | | |
| Group D | IL-6 | 500 ng/mL | Rabbit anti- | HRP Rabbit | Room |
| | | | mouse IL-6 | anti-goal IgG | temperature |
| | | | (Abcam | H + L (Bethyl | for one hour |
| | | | #ab9730) | #A50-100) | |
| Group E | IL-12/23 | 500 ng/ml | Goat anti- | HRP Rabbit | Room |
| | p40 | | mouse IL- | anti-goat IgG | temperature |
| | | | 12p40 IgG | H + L (Bethyl | for one hour |
| | | | (Abgent #Af1561a | #A50-100) | |
| Group E | IL-23 | 500 ng/mL | Rabbit anti- | HRP Goat anti- | Room |
| | | | mouse IL-23 | rabbit H + L | temperature |
| | | | (Abbiotec | (Bethyl | for one hour |
| | | | #251562) | #A120-101P) | |
| Group E | IL-12p70 | 500 ng/mL | Rat anti-mouse | HRP Goat anti- | Room |
| | | | IL-12p70 | Rat IgG H + L | temperature |
| | | | (R&D systeams | (Bethyl #A50-100P) | for one hour |
| | | | #MAB419) | | |
| Group E | MAdCAM-1 | 500 ng/mL | Goat anti-mouse | HRF Rabbit | Room |
| | | | MADCAM-1 | anti-goat IgG | temperature |
| | | | IgG (LSBio | H + L (Bethyl | for one hour |
| | | | #LS-C150068) | #A50-100) | |

### ELISA Development

Group A was immunized with PT-gliadin peptides and the group sera were evaluated using ELISAs for binding to PT-gliadin, gliadin in urea, and gliadin in acetic acid in order to compare different forms of gliadin that may display immunogenic epitopes to different extents. Group B was immunized with a gliadin 33-mer peptide and the group sera were also evaluated using a gliadin 33-mer, gliadin in urea, and gliadin in acetic acid ELISAs. A polyclonal Rabbit anti-gliadin (Sigma #G9144) was used as reference curve in all of the gliadin ELISA formats. All four standard curves looked similar, all had low background, showed dose-related antibody binding, reached high absorbance at high antibody concentrations, and had low variability between duplicate wells. The reference curves for IL-6, IL-6Rα, IL-12/23p40, and MAdCAM-1 used in Group C, D, and E ELISAs showed similar results.

Table 23 provides a summary of the maximal antibody titers of the individual and pooled calf sera from each of the five Groups tested in various ELISAs.

TABLE 23

| Group | | | | | | |
|---|---|---|---|---|---|---|
| # | Immunogen | ELISA | Maximal Antibody Titer | | | |
| | | | Calf #26 | Calf #28 | Calf #33 | Pool A |
| A | PT-Gliadin | PT-Gliadin | 1336 | 5871 | 376 | 2448 |
| | | Gliadin in | 823 | 1469 | 267 | 833 |
| | | Acetic acid | | | | |
| | | Gliadin in | 902 | 1528 | 218 | 826 |
| | | Urea | | | | |
| | | | Calf #22 | Calf #30 | Calf #32 | Pool B |
| B | Gliadin | Gliadin | 2301 | 233 | 1950 | 1410 |
| | 33-mer | 33-mer | | | | |
| | | Gliadin in | 297 | 504 | 642 | 414 |
| | | Acetic acid | | | | |
| | | Gliadin in | 326 | 358 | 888 | 506 |
| | | Urea | | | | |
| | | | Calf #21 | Calf #27 | Calf #34 | Pool C |
| C | IL-6Rα | IL-6Rα | 2609 | 3163 | 9017 | 6764 |
| | | | Calf #29 | Calf #35 | Calf #36 | Pool D |
| D | IL-6 + | IL-6 | 2301 | 233 | 1950 | 1410 |
| | IL-6Rα | IL-6Rα | 6007 | 9029 | 6983 | 7248 |
| | | | Calf #23 | Calf #24 | Calf #31 | Pool E |
| E | IL-6Rα + | IL-6Rα | 752 | 1557 | 3926 | 1490 |
| | MAdCAM-1 + | MAdCAM-1 | 4520 | 6368 | 13651 | 7789 |
| | IL-12/23p40 | IL-12/23p40 | 7724 | 8878 | 22828 | 13097 |
| | | IL-12p70 | 7325 | 4694 | 5663 | 3619 |
| | | IL-23 | 5280 | 2674 | 3569 | 2747 |

In Group A, calf 28 was a high responder after the second, third and fourth immunizations, while calf 33 did not show a clear response to PT-gliadin immunization at any time point. Calf 26 was a weak responder to PTgliadin, with antibody titers slightly elevated after 2 immunizations. Pooled sera of calves 26, 28 and 33 had intermediate antibody titers relative to the individual calves as would be expected.

Group B sera was analyzed to determine if antibodies would bind to the Gliadin 33-mer peptide coated plates, as well as whole gliadin prepared in acetic acid and in urea. There were significantly higher titers for calves 22 and 32 to gliadin 33-mer compared to gliadin in either acetic acid or urea, suggesting that the dominant immunogenic epitopes on the 33-mer peptide may not be exposed or conformationally present on either form of the gliadin molecule. Calf 30 sera had little or no antibody titers vs any of the three gliadin preparations.

Group C sera were analyzed using a direct ELISA to IL-6Rα, using the same antigen in the ELISA as was used in the immunization of the calves. Calf 34 was a high responder in the ELISA while calves 21 and 27 showed lower antibody titers.

Group D calves were immunized with both IL-6 and IL-6Rα, and sera were analyzed using separate direct ELISAs to IL-6 and IL-6Rα using the same antigens in the ELISAs as were used in the immunization of the calves. Calf 36 had the highest peak antibody response among the three calves after the second immunization for both IL-6 and IL-6R□, but the antibody response decreased with subsequent immunizations, particularly vs IL-6. The antibody responses of the three calves vs IL-6 had a greater range than those vs IL-6Rα, although all three calves clearly responded to both antigens when they were administered together. The antibody titers of the pooled Group D sera vs IL-6Ra were comparable to those from pooled Group C sera, suggesting that the presence of IL-6 in Group D did not greatly affect the response to IL-6R□.

Group E calves were immunized with a cocktail of IL-6Rα, IL-12/IL-23p40, and MAdCAM-1. The sera were analyzed in separate direct ELISAs for binding to IL-6Rα, IL-12/1L-23p40, MAdCAM-1, IL-12p70, and IL-23. Calf 31 had the highest antibody response to IL-6Rα, IL-12/1L-23p40, and MAdCAM-1. However, the magnitude of the responses to IL-6R□□ was generally smaller than those vs IL-6R□□ in Groups C and D. Given the variability in immune responses between individual animals, these lower titers to IL-6R□□ may simply reflect that variability. All 3 calves had comparable antibody titers to the heterodimeric molecules IL-12p70 and IL-23, although the titers were lower than vs the IL-12/23p40 chain alone.

These results collectively show the feasibility of generating therapeutic bovine antibodies to Gliadin, IL-6Rα, IL-6 and MAdCAM-1 for evaluation in animal models or human therapy of celiac disease, IBD or other GI indications.

### Example 13—DNA Vaccination for Generation of Bovine Antibodies

A strong antibody response to a protein antigen can be induced by vaccination of cattle with a DNA vector containing a gene encoding the desired antigen (see, e.g., van Drunen Little-van den Hurk et al., Clin Vaccine Immuno/2013; 20:166-73). Individual animals are injected in the right gluteus maximus muscle with 1.5 mg of plasmid DNA in 1 mL, followed by application of a 250-V/cm electrical field for 400 ms at a 10% duty cycle, using a TriGrid electrode array (Ichor Medical Systems). The immunizations are repeated two or three times, 21 days apart, and are expected to induce a significant antibody response.

### Example 14—Generation of Hybridomas Producing Monoclonal Bovine Antibodies Following Immunization

Hybridomas can be generated from immunized cows to establish bovine antibody-secreting cell lines, essentially as described in U.S. Pat. No. 5,026,646.

Right prescapular, left prescapular, and right prefernoral superficial lymph nodes of cows immunized as described in Examples 11 and 12 are surgically removed under general anesthesia, sectioned, and passed through a 80-mesh sieve. Extracted cells are washed and used for fusion. Fusions are performed with modified version of the PEG protocol (Van Deusen et al., Am Assoc Vet Lab Diagnost 24^{th} Annual Proc 1981:211-228) using myeloma cell:lymph node cell ratios of approximately 1, and seeded onto 96-well plates. Cells are cultured at a 1:1 ratio of DMEM and high glucose with 10% horse serum to conditioned media from fusion partner cultures. Fusion partner selection for methotrexate sensitivity is achieved by passage in media containing 6-thioguanine and 8-azaguanine.

SP2/0 murine myeloma cells are fused with right prescapular calf lymph nodes cells in a 3:1 mixture to generate bovine Ig secreting primary cell lines. After secretion ceases, the lines are selected for methotrexate sensitivity by passage in media containing 6-thioguanine and 8-azaguanine, Methotrexate-sensitive bovine:murine heterohybridomas are combined in a 1:1 ratio and mixed with lymph node cells from the immunized and extracted left prescapular lymph node from the same calf used previously at a 1:1 ratio. This fusion results in transiently bovine Ig secreting cell lines, which are then selected for methotrexate sensitivity once secretion ceases. Cells from the methotrexate-sensitive bovine×murine heterohybridomas are combined in a 1:1 ratio and fused with fresh lymph node cells from an immunized, right prefernoral lymph node from the same calf as before at a 1:2 mixture, respectively. The fusion results in bovine×murine primary heterohybridomas that secrete bovine antibodies. Stable heterohybridomas secreting antibodies having the function of interest (e.g., binding to TNF) are identified, cloned, and subcloned.

The heterohybridomas can be further screened for those which produce IgG1 antibodies by sequencing using routine methods.

### Example 15—Single B Cell Sequencing and Recombinant Antibody Production

Single B cells are sequenced essentially as described by Tiller et al. (J Immunol Methods 2008; 329:122-4). Mononuclear cells are isolated from peripheral venous blood or serum of cows immunized as described in Examples 11 and 12, purified by Ficoll-Paque density gradient centrifugation, and optionally followed by enrichment of B cells using anti-CD19 magnetic beads. Single mononuclear cells are sorted by flow cytometry into 96-well PCR plates. cDNA is synthesized directly in each well. Total RNA from single cells are reverse transcribed with random hexamers using the Superscript III reverse transcriptase kit, and IgG, Igλ and Igκ V transcripts are amplified with gene-specific primers using the isolated cDNA as template. Aliquots of the V_{H}, Vκ and Vλ chain PCR products are purified and sequenced. Sequences are analyzed by IgBLAST comparison with Gen Bank to identify germline V(D)J gene segments with highest identity.

PCR products are purified using the Qia-Quick 96 PCR Purification Kit (Qiagen). PCR samples are digested and ligated into the multiple cloning site of human Igγ1, Igκ and Igλ expression vectors. Ligation products are transformed into competent DH10B bacteria cells, and colonies are PCR screened for the presence of bands of the expected size. Plasmid DNA is isolated from bacterial cultures using QIAprep Spin columns (Qiagen) and purified.

Antibodies are produced by transiently transfecting the purified plasmids into HEK293 cells or 293T cells cultured in 150 mm plates. Six days after transfection, culture supernatants are harvested and antibodies are purified using Protein G beads. Recombinant antibody concentrations are then determined by ELISA.

### Example 16—Selection of Recombinant Bovine Monoclonal IgG1 Antibodies

Antibodies produced according to Example 14 or 15 are sequenced to determine whether they have the structural properties that confer resistance to proteases as described herein, and tested for functional activity (e.g., binding to target antigen) and stability using methods described in the preceding Examples. Further, as discussed supra, potential protease cut sites in the antigen-binding region are also considered when generating stable bovine IgG1 antibodies. Thus, the antibodies are tested for both antigen binding and pancreatin stability using the methods described in Examples 2 and 10 to confirm they retain antigen binding and stability from pancreatin digestion.

The heavy and light chains of antibodies found to have the desired properties (both structural and functional) are cloned into expression vectors, expressed, harvested, and purified using standard recombinant methods.

### Example 17—Construct Design for Fully Bovine Recombinant Monoclonal Antibodies and Bovinized Antibodies

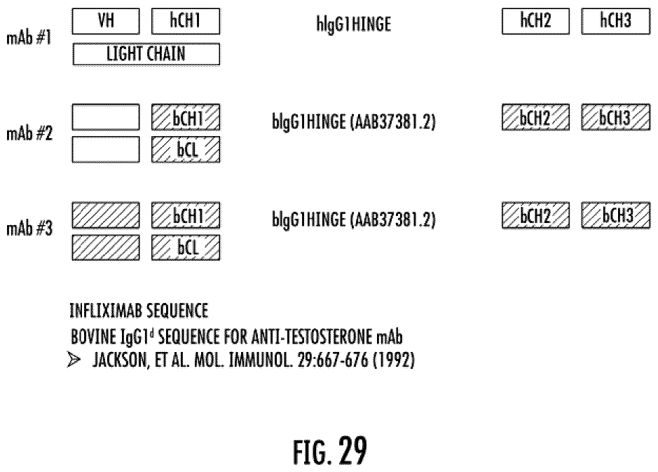

A parent antibody may be modified to impart the features of a bovine early colostral antibody such that the modified antibody has enhanced resistance to protease digestion. FIG. 29 shows the various constructs that can made (using infliximab as an exemplary parent antibody) and inserted into expression vectors in host cells that may be any mammalian host cell or a ruminant mammary epithelial cell line such as MAC-T for expression.

TABLE 24

| Description | Amino acid sequence |
|---|---|
| Infliximab Fab sequence - light | DILLTQSPAILSVSPGERVSFSCRASQFVGSSIHWYQQRINGSPRLLIKYASESMSG |
| chain | IPSRFSGSGSGTDFTLSINTVESEDIADYYCQQSHSWPFTFGSGTNLEVKRTVAAPS |
| (SEQ ID NO: 26) | VFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSENRGEC |
| Infliximab Fab sequence - heavy | EVKLEESGGGLVQPGGSMKLSCVASGFIFSNHWMNWVRQSPEKGLEWVAEIRSKSIN |
| chain | SATHYAESVKGRFTISRDDSKSAVYLQMTDLRTEDTGVYYCSRNYYGSTYDYWGQGT |
| (SEQ ID NO: 27) | TLTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKT |
| Infliximab sequence heavy chain | EVKLEESGGGLVQPGGSMKLSCVASGFIFSNHWMNWVRQSPEKGLEWVAEIRSKSIN |
| construct #1 | SATHYAESVKGRFTISRDDSKSAVYLQMTDLRTEDTGVYYCSRNYYGSTYDYWGQGT |
| (SEQ ID NO: 28) | TLTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKA |
| | KGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGK |
| Infliximab w/Bovine Fc | EVKLEESGGGLVQPGGSMKLSCVASGFIFSNHWMNWVRQSPEKGLEWVAEIRSKSIN |
| Heavy chain construct #2 | SATHYAESVKGRFTISRDDSKSAVYLQMTDLRTEDTGVYYCSRNYYGSTYDYWGQGT |
| (SEQ ID NO: 29) | TLTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHT |
| | CPPCPAPELLGGPSVFIFPPKPKDTLTISGTPEVTCVVVDVGHDDPEVKFSWFVDDV |
| | EVNTATTKPREEQFNSTYRVVSALRIQHQDWTGGKEFKCKVHNEGLPAPIVRTISRT |
| | KGPAREPQVYVLAPPQEELSKSTVSLTCMVTSFYPDYIAVEWQRNGQPESEDKYGTT |
| | PPQLDADGSYFLYSRLRVDRNSWQEGDTYTCVVMHEALHNHYTQKSTSKSAGK |
| Infliximab with Bovine Fc IgG1 | EVKLEESGGGLVQPGGSMKLSCVASGFIFSNHWMNWVRQSPEKGLEWVAEIRSKSIN |
| hinge | SATHYAESVKGRFTISRDDSKSAVYLQMTDLRTEDTGVYYCSRNYYGSTYDYWGQGT |
| Heavy chain construct #3 | TLTVSSASTKGPSVFPLAPSSKSTSGGTAALGCLVKDYFPEPVTVSWNSGALTSGVH |
| (SEQ ID NO: 30) | TFPAVLQSSGLYSLSSVVTVPSSSLGTQTYICNVNHKPSNTKVDKKVEPKSCKTTCD |
| | CCPPPELPGGPSVFIFPPKPKDTLTISGTPEVTCVVVDVGHDDPEVKFSWFVDDVEV |
| | NTATTKPREEQFNSTYRVVSALRIQHQDWTGGKEFKCKVHNEGLPAPIVRTISRTKG |
| | PAREPQVYVLAPPQEELSKSTVSLTCMVTSFYPDYIAVEWQRNGQPESEDKYGTTPP |
| | QLDADGSYFLYSRLRVDRNSWQEGDTYTCVVMHEALHNHYTQKSTSKSAGK |
| Infliximab with bovine Fc, IgG2 | EVKLEESGGGLVQPGGSMKLSCVASGFIFSNHWMNWVRQSPEKGLEWVAEIRSKSIN |
| hinge | SATHYAESVKGRFTISRDDSKSAVYLQMTDLRTEDTGVYYCSRNYYGSTYDYWGQGT |
| Heavy Chain construct #5 | TLTVSSASTKGPSVFPLAPSSKSTSGGTAALGCLVKDYFPEPVTVSWNSGALTSGVH |
| (SEQ ID NO: 31) | TFPAVLQSSGLYSLSSVVTVPSSSLGTQTYICNVNHKPSNTKVDKKVEPKSCKIDCS |
| | KCHNQPCVREPSVFIFPPKPKDTLTISGTPEVTCVVVDVGHDDPEVKFSWFVDDVEV |
| | NTATTKPREEQFNSTYRVVSALRIQHQDWTGGKEFKCKVHNEGLPAPIVRTISRTKG |
| | PAREPQVYVLAPPQEELSKSTVSLTCMVTSFYPDYIAVEWQRNGQPESEDKYGTTPP |
| | QLDADGSYFLYSRLRVDRNSWQEGDTYTCVVMHEALHNHYTQKSTSKSAGK |
| Infliximab w/bovine Fc, IgG3 hinge | EVKLEESGGGLVQPGGSMKLSCVASGFIFSNHWMNWVRQSPEKGLEWVAEIRSKSIN |
| Heavy Chain construct #6 | SATHYAESVKGRFTISRDDSKSAVYLQMTDLRTEDTGVYYCSRNYYGSTYDYWGQGT |
| (SEQ ID NO: 32) | TLTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCRPVPT |
| | TPKTTIPPGKPTTQESEVEKTPCQCSKCPEPLGGLSVFIFPPKPKDTLTISGTPEVT |
| | CVVVDVGHDDPEVKFSWFVDDVEVNTATTKPREEQFNSTYRVVSALRIQHQDWTGGK |
| | EFKCKVHNEGLPAPIVRTISRTKGPAREPQVYVLAPPQEELSKSTVSLTCMVTSFYP |
| | DYIAVEWQRNGQPESEDKYGTTPPQLDADGSYFLYSRLRVDRNSWQEGDTYTCVVMH |
| | EALHNHYTQKSTSKSAGK |
| Infliximab sequence light chain with | DILLTQSPAILSVSPGERVSFSCRASQFVGSSIHWYQQRINGSPRLLIKYASESMSG |
| bovine kappa constant light domain | IPSRFSGSGSGTDFTLSINTVESEDIADYYCQQSHSWPFTFGSGTNLEVKRTVAAPS |
| (bovine constant light chain (~75%) | VFLFKPSDEQLKTGTVSVVCLVNDFYPKDINVKWKVDGVTQSSSNFQNSFTDQDSKK |
| with highest homology to infliximab | STYSLSSILTLPSSEYQSHNAYTCEVSHKSLTTALVKSFSKNEC |
| constant light chain was chosen) | |
| (SEQ ID NO: 33) | |
| Infliximab sequence light chain with | DILLTQSPAILSVSPGERVSFSCRASQFVGSSIHWYQQRINGSPRLLIKYASESMSG |
| bovine lambda constant light chain | IPSRFSGSGSGTDFTLSINTVESEDIADYYCQQSHSWPFTFGSGTNLEVKGQPKSPP |
| domain (bovine constant light chain | SVTLFPPSTEELNGNKATLVCLISDFYPGSVTVVWKADGSTITRNVETTRASKQSNS |
| domain with the highest abundance | KYAASSYLSLTSSDWKSKGSYSCEVTHEGSTVTKTVKPSECS |
| in AVX-470 and other colostral IgG | |
| preparations was chosen) | |
| (SEQ ID NO: 34) | |
| Chimeric infliximab with bovine | EVKLEESGGGLVQPGGSMKLSCVASGFIFSNHWMNWVRQSPEKGLEWVAEIRSKSIN |
| IgG1 constant region (CH1, hinge, | SATHYAESVKGRFTISRDDSKSAVYLQMTDLRTEDTGVYYCSRNYYGSTYDYWGQGT |
| and Fc domain sequences) (bovine | TLTVSSASTTAPKVYPLSSCCGDKSSSTVTLGCLVSSYMPEPVTVTWNSGALKSGVH |
| sequences are underlined)-heavy | TFPAVLQSSGLYSLSSMVTVPGSTSGQTFTCNVAHPASSTKVDKAVDPTCKPSPCDC |
| chain | CPPPELPGGPSVFIFPPKPKDTLTISGTPEVTCVVVDVGHDDPEVKFSWFVDDVEVN |
| (SEQ ID NO: 35) | TATTKPREEQFNSTYRVVSALRIQHQDWTGGKEFKCKVHNEGLPAPIVRTISRTKGP |
| | AREPQVYVLAPPQEELSKSTVSLTCMVTSFYPDYIAVEWQRNGQPESEDKYGTTPPQ |
| | LDADSSYFLYSKLRVDRNSWQEGDTYTCVVMHEALHNHYTQKSTSKSAGK |
| Chimeric infliximab with bovine | DILLTQSPAILSVSPGERVSFSCRASQFVGSSIHWYQQRINGSPRLLIKYASESMSG |
| IgG1 constant region (CH1, hinge, | IPSRFSGSGSGTDFTLSINTVESEDIADYYCQQSHSWPFTFGSGTNLEVKRSDAEPS |
| and Fc domain sequences) (bovine | VFLFKPSDEQLKTGTVSVVCLVNDFYPKDINVKWKVDGVTQSSSNFQNSFTDQDSKK |
| sequences are underlined)-light | STYSLSSILTLPSSEYQSHNAYTCEVSHKSLTTALVKSFSKNEC |
| chain | |
| (SEQ ID NO: 36) | |

Heavy and light chain sequences of exemplary bovinized antibodies are provided below (bovine sequences are underlined).

TABLE 25

| Description | Sequence |
|---|---|
| Bovinized anti-a4b7 integrin | QVQLQQPGAELVKPGTSVKLSCKGYGYTFTSYWMHWVKQRPGQGLEWIGEIDPSESNT |
| antibody (cross-reacts with mouse | NYNQKFKGKATLTVDISSSTAYMQLSSLTSEDSAVYYCARGGYDGWDYAIDYWGQGTS |
| and human a4b7 integrin; PDB ID: | VTVSSASTTAPKVYPLSSCCGDKSSSTVTLGCLVSSYMPEPVTVTWNSGALKSGVHTF |
| 3V4P, V domain pI = 6.72)-heavy | PAVLQSSGLYSLSSMVTVPGSTSGQTFTCNVAHPASSTKVDKAVDPTCKPSPCDCCPP |
| chain | PELPGGPSVFIFPPKPKDTLTISGTPEVTCVVVDVGHDDPEVKFSWFVDDVEVNTATT |
| (SEQ ID NO: 37) | KPREEQFNSTYRVVSALRIQHQDWTGGKEFKCKVHNEGLPAPIVRTISRTKGPAREPQ |
| | VYVLAPPQEELSKSTVSLTCMVTSFYPDYIAVEWQRNGQPESEDKYGTTPPQLDADSS |
| | YFLYSKLRVDRNSWQEGDTYTCVVMHEALHNHYTQKSTSKSAGK |
| Bovinized anti-a4b7 integrin | DVVVTQTPLSLPVSFGDQVSISCRSSQSLAKSYGNTYLSWYLHKPGQSPQLLIYGISN |
| antibody (cross-reacts with mouse | RFSGVPDRFSGSGSGTDFTLKISTIKPEDLGMYYCLQGTHQPYTFGGGTKLEIKRSDA |
| and human a4b7 integrin; PDB ID: | EPSVFLFKPSDEQLKTGTVSVVCLVNDFYPKDINVKWKVDGVTQSSSNFQNSFTDQDS |
| 3V4P, V domain pI = 6.72)-light | KKSTYSLSSILTLPSSEYQSHNAYTCEVSHKSLTTALVKSFSKNEC |
| chain | |
| (SEQ ID NO: 38) | |
| Bovinized anti-IL-12 antibody | EVQLVQSGAEVKKPGESLKISCKGSGYSFTTYWLGWVRQMPGKGLDWIGIMSPVDSDI |
| (PBD ID: 3HMW, V domain pI = | RYSPSFQGQVTMSVDKSITTAYLQWNSLKASDTAMYYCARRRPGQGYFDFWGQGTLVT |
| 8.74)-heavy chain | VSSASTTAPKVYPLSSCCGDKSSSTVTLGCLVSSYMPEPVTVTWNSGALKSGVHTFPA |
| (SEQ ID NO: 39) | VLQSSGLYSLSSMVTVPGSTSGQTFTCNVAHPASSTKVDKAVDPTCKPSPCDCCPPPE |
| | LPGGPSVFIFPPKPKDTLTISGTPEVTCVVVDVGHDDPEVKFSWFVDDVEVNTATTKP |
| | REEQFNSTYRVVSALRIQHQDWTGGKEFKCKVHNEGLPAPIVRTISRTKGPAREPQVY |
| | VLAPPQEELSKSTVSLTCMVTSFYPDYIAVEWQRNGQPESEDKYGTTPPQLDADSSYF |
| | LYSKLRVDRNSWQEGDTYTCVVMHEALHNHYTQKSTSKSAGK |
| Bovinized anti-IL-12 antibody | DIQMTQSPSSLSASVGDRVTITCRASQGISSWLAWYQQKPEKAPKSLIYAASSLQSGV |
| (PBD ID: 3HMW, V domain pI = | PSRFSGSGSGTDFTLTISSLQPEDFATYYCQQYNIYPYTFGQGTKLEIKRSDAEPSVF |
| 8.74)-light chain | LFKPSDEQLKTGTVSVVCLVNDFYPKDINVKWKVDGVTQSSSNFQNSFTDQDSKKSTY |
| (SEQ ID NO: 40) | SLSSILTLPSSEYQSHNAYTCEVSHKSLTTALVKSFSKNEC |
| Bovinized anti-MAdCAM-1 | QVQLVQSGAEVKKPGASVKVSCKASGYTFTSYGINWVRQAPGQGLEWMGWISVYSGNT |
| antibody (PBD ID: 4HCR, V | NYAQKVQGRVTMTADTSTSTAYMDLRSLRSDDTAVYYCAREGSSSSGDYYYGMDVWGQ |
| domain pI = 7.6)-heavy chain | GTTVTVSSASTTAPKVYPLSSCCGDKSSSTVTLGCLVSSYMPEPVTVTWNSGALKSGV |
| (SEQ ID NO: 41) | HTFPAVLQSSGLYSLSSMVTVPGSTSGQTFTCNVAHPASSTKVDKAVDPTCKPSPCDC |
| | CPPPELPGGPSVFIFPPKPKDTLTISGTPEVTCVVVDVGHDDPEVKFSWFVDDVEVNT |
| | ATTKPREEQFNSTYRVVSALRIQHQDWTGGKEFKCKVHNEGLPAPIVRTISRTKGPAR |
| | EPQVYVLAPPQEELSKSTVSLTCMVTSFYPDYIAVEWQRNGQPESEDKYGTTPPQLDA |
| | DSSYFLYSKLRVDRNSWQEGDTYTCVVMHEALHNHYTQKSTSKSAGK |
| Bovinized anti-MAdCAM-1 | DIVMTQTPLSLSVTPGQPASISCKSSQSLLHTDGTTYLYWYLQKPGQPPQLLIYEVSN |
| antibody (PBD ID: 4HCR, V | RFSGVPDRFSGSGSGTDFTLKISRVEAEDVGIYYCMQNIQLPWTFGQGTKVEIKRSDA |
| domain pI = 7.6)-light chain | EPSVFLFKPSDEQLKTGTVSVVCLVNDFYPKDINVKWKVDGVTQSSSNFQNSFTDQDS |
| (SEQ ID NO: 42) | KKSTYSLSSILTLPSSEYQSHNAYTCEVSHKSLTTALVKSFSKNEC |
| Bovinized anti-IL-23 antibody | EVQLQQSGPELVKPGASVKMSCKASGYTFTSNVMHWVKQKPGQGLEWIGYINPYNDGT |
| (PBD ID: 3D85, V domain pI = | KYNEKFKGKATLTSDKSSSTAYMELSSLTSEDSAVYYCARNWDVAYWGQGTLVTVSAA |
| 8.38)-heavy chain | STTAPKVYPLSSCCGDKSSSTVTLGCLVSSYMPEPVTVTWNSGALKSGVHTFPAVLQS |
| (SEQ ID NO: 43) | SGLYSLSSMVTVPGSTSGQTFTCNVAHPASSTKVDKAVDPTCKPSPCDCCPPPELPGG |
| | PSVFIFPPKPKDTLTISGTPEVTCVVVDVGHDDPEVKFSWFVDDVEVNTATTKPREEQ |
| | FNSTYRVVSALRIQHQDWTGGKEFKCKVHNEGLPAPIVRTISRTKGPAREPQVYVLAP |
| | PQEELSKSTVSLTCMVTSFYPDYIAVEWQRNGQPESEDKYGTTPPQLDADSSYFLYSK |
| | LRVDRNSWQEGDTYTCVVMHEALHNHYTQKSTSKSAGK |
| Bovinized anti-IL-23 antibody | DIVMTQSPATLSVTPGDRVSLSCRASQSISDYLHWYRQKSHESPRLLIKYASQSISGI |
| (PBD ID: 3D85, V domain pI = | PSRFSGSGSGSDFTLSINSVEPEDVGVYYCQNGHSFPFTFGSGTKLEIKRSDAEPSVF |
| 8.38)-light chain | LFKPSDEQLKTGTVSVVCLVNDFYPKDINVKWKVDGVTQSSSNFQNSFTDQDSKKSTY |
| (SEQ ID NO: 44) | SLSSILTLPSSEYQSHNAYTCEVSHKSLTTALVKSFSKNEC |
| Bovinized anti-EGFR/HER3 | EVQLVESGGGLVQPGGSLRLSCAASGFTLSGDWIHWVRQAPGKGLEWLGEISAAGGYT |
| bispecific antibody (PBD ID: | DYADSVKGRFTISADTSKNTAYLQMNSLRAEDTAVYYCARESRVSFEAAMDYWGQGTL |
| 3P0V, V domain pI = 6.13)-heavy | VTVSSASTTAPKVYPLSSCCGDKSSSTVTLGCLVSSYMPEPVTVTWNSGALKSGVHTF |
| chain | PAVLQSSGLYSLSSMVTVPGSTSGQTFTCNVAHPASSTKVDKAVDPTCKPSPCDCCPP |
| (SEQ ID NO: 45) | PELPGGPSVFIFPPKPKDTLTISGTPEVTCVVVDVGHDDPEVKFSWFVDDVEVNTATT |
| | KPREEQFNSTYRVVSALRIQHQDWTGGKEFKCKVHNEGLPAPIVRTISRTKGPAREPQ |
| | VYVLAPPQEELSKSTVSLTCMVTSFYPDYIAVEWQRNGQPESEDKYGTTPPQLDADSS |
| | YFLYSKLRVDRNSWQEGDTYTCVVMHEALHNHYTQKSTSKSAGK |
| Bovinized anti-EGFR/HER3 | DIQMTQSPSSLSASVGDRVTITCRASQDLATDVAWYQQKPGKAPKLLIYSASFLYSGV |
| bispecific antibody (PBD ID: | PSRFSGSGSGTDFTLTISSLQPEDFATYYCQQSEPEPYTFGQGTKVEIKRSDAEPSVF |
| 3P0V, V domain pI = 6.13)-light | LFKPSDEQLKTGTVSVVCLVNDFYPKDINVKWKVDGVTQSSSNFQNSFTDQDSKKSTY |
| chain | SLSSILTLPSSEYQSHNAYTCEVSHKSLTTALVKSFSKNEC |
| (SEQ ID NO: 46) | |
| Bovinized anti-CD25 antibody | EVQLQQSGTVLARPGASVKMSCKASGYSFTRYWMHWIKQRPGQGLEWIGAIYPGNSDT |
| (PBD ID: 3P0V, V domain pI = | SYNQKFEGKAKLTAVTSASTAYMELSSLTHEDSAVYYCSRDYGYYFDFWGQGTTLTVS |
| 6.13)-heavy chain | SASTTAPKVYPLSSCCGDKSSSTVTLGCLVSSYMPEPVTVTWNSGALKSGVHTFPAVL |
| (SEQ ID NO: 47) | QSSGLYSLSSMVTVPGSTSGQTFTCNVAHPASSTKVDKAVDPTCKPSPCDCCPPPELP |
| | GGPSVFIFPPKPKDTLTISGTPEVTCVVVDVGHDDPEVKFSWFVDDVEVNTATTKPRE |
| | EQFNSTYRVVSALRIQHQDWTGGKEFKCKVHNEGLPAPIVRTISRTKGPAREPQVYVL |
| | APPQEELSKSTVSLTCMVTSFYPDYIAVEWQRNGQPESEDKYGTTPPQLDADSSYFLY |
| | SKLRVDRNSWQEGDTYTCVVMHEALHNHYTQKSTSKSAGK |
| Bovinized anti-CD25 antibody | QIVSTQSPAIMSASPGEKVTMTCSASSSRSYMQWYQQKPGTSPKRWIYDTSKLASGVP |
| (PBD ID: 3P0V, V domain pI = | ARFSGSGSGTSYSLTISSMEAEDAATYYCHQRSSYTFGGGTKLEIKRSDAEPSVFLFK |
| 6.13)-light chain | PSDEQLKTGTVSVVCLVNDFYPKDINVKWKVDGVTQSSSNFQNSFTDQDSKKSTYSLS |
| (SEQ ID NO: 48) | SILTLPSSEYQSHNAYTCEVSHKSLTTALVKSFSKNEC |
| Bovinized anti-IL-6R_antibody- | EVQLVESGGGLVQPGRSLRLSCAASRFTFDDYAMHWVRQAPGKGLEWVSGISWNSGRI |
| heavy chain | GYADSVKGRFTISRDNAENSLFLQMNGLRAEDTALYYCAKGRDSFDIWGQGTMVTVSS |
| (SEQ ID NO: 49) | ASTTAPKVYPLSSCCGDKSSSTVTLGCLVSSYMPEPVTVTWNSGALKSGVHTFPAVLQ |
| | SSGLYSLSSMVTVPGSTSGQTFTCNVAHPASSTKVDKAVDPTCKPSPCDCCPPPELPG |
| | GPSVFIFPPKPKDTLTISGTPEVTCVVVDVGHDDPEVKFSWFVDDVEVNTATTKPREE |
| | QFNSTYRVVSALRIQHQDWTGGKEFKCKVHNEGLPAPIVRTISRTKGPAREPQVYVLA |
| | PPQEELSKSTVSLTCMVTSFYPDYIAVEWQRNGQPESEDKYGTTPPQLDADSSYFLYS |
| | KLRVDRNSWQEGDTYTCVVMHEALHNHYTQKSTSKSAGK |
| Bovinized anti-IL-6R_antibody- | DIQMTQSPSSVSASVGDRVTITCRASQGISSWLAWYQQKPGKAPKLLIYGASSLESGV |
| light chain | PSRFSGSGSGTDFTLTISSLQPEDFASYYCQQANSFPYTFGQGTKLEIKRSDAEPSVF |
| (SEQ ID NO: 50) | LFKPSDEQLKTGTVSVVCLVNDFYPKDINVKWKVDGVTQSSSNFQNSFTDQDSKKSTY |
| | SLSSILTLPSSEYQSHNAYTCEVSHKSLTTALVKSFSKNEC |

### Minimal Bovinization

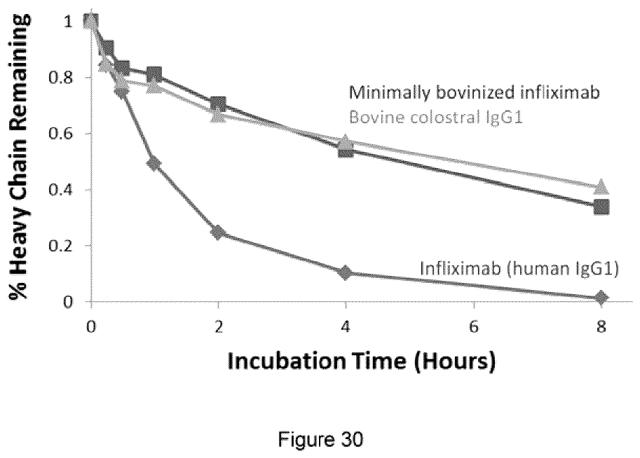

Presented below are minimally bovinized human IgG1, 2, and 4 constant region sequences which exhibit protease resistance and be incorporated into parent antibodies (e.g., combined with the variable regions of the parent antibody) to create highly stable, digestion resistant antibodies which can be administered orally. The constant regions of the parent antibody are replaced with the minimally bovinized sequences using routine recombinant techniques. Protease resistance can then be tested using the art-recognized methods described herein. For instance, the minimally bovinized human IgG1 construct was purified and tested by pancreatin digestion, as described in Example 3, and shown to have comparable protease stability to bovine colostral IgG1 and significantly greater protease stability than the parent human IgG1 molecule (infliximab) (FIG. 30).

TABLE 26
Minimally bovinized human IgG1

| | |
|---|---|
| Human IgG1 | ASTKGPSVFPLAPSSKSTSGGTAALGCLVKDYFPEPVTVSWNSGALTSGVHTFPAVLQSSGLYSLSSVVTVP |
| (SEQ ID NO: 51) | SSSLGT |
| Bovine IgGld | ASTTAPKVYPLSSCCGDKSSSTVTLGCLVSSYMPEPVTVTWNSGALKSGVHTFPAVLQSSGLYSLSSMVTVP |
| (CAA44699.1) | GSTSG |
| (SEQ ID NO: 52) | |
| Minimally | ASTKGPSVFPLSSCCGDKSGGTAALGCLVKDYFPEPVTVSWNSGALTSGVHTFPAVLQSSGLYSLSSVVTVP |
| bovinized IgG1 | SSSLGT |
| (SEQ ID NO: 53) | |
| Human IgG1 | QTYICNVNHKPSNTKVDKKVEPKSCDKTHTCPPCPAPELLGGPSVFLFPPKPKDTLMISRTPEVTCVVVDVS |
| (SEQ ID NO: 54) | HEDPEV |
| Bovine IgGld | QTFTCNVAHPASSTKVDKAVDP.TC.KPSPCDCCPPPELPGGPSVFIFPPKPKDTLTISGTPEVTCVVVDVG |
| (CAA44699.1) | HDDPEV |
| (SEQ ID NO: 55) | |
| Minimally | QTYICNVNHKPSNTKVDKAVDP.TC.KPSPCDCCPPPELLGGPSVFLFPPKPKDTLMISRTPEVTCVVVDVS |
| bovinized IgG1 | HEDPEV |
| (SEQ ID NO: 56) | |

TABLE 27
Minimally bovinized human IgG2

| | |
|---|---|
| Human IgG2 | ASTKGPSVFPLAPCSRSTSESTAALGCLVKDYFPEPVTVSWNSGALTSGVHTFPAVLQSSGLYSLSSVVTVP |
| (AAB59393.1) | SSNFGT |
| (SEQ ID NO: 57) | |
| Bovine IgG1d | ASTTAPKVYPLSSCCGDKSSSTVTLGCLVSSYMPEPVTVTWNSGALKSGVHTFPAVLQSSGLYSLSSMVTVP |
| (CAA44699.1) | GSTSG |
| (SEQ ID NO: 58) | |
| Minimally | ASTKGPSVFPLSSCCGDKSESTAALGCLVKDYFPEPVTVSWNSGALTSGVHTFPAVLQSSGLYSLSSVVTVP |
| bovinized IgG2 | SSNFGT |
| (SEQ ID NO: 59) | |
| Human IgG2 | QTYTCNVDHKPSNTKVDKTVERKCCVE.CPPCPAP.PVAGPSVFLFPPKPKDTLMISRTPEVTCVVVDVSHE |
| (AAB59393.1) | DPEV |
| (SEQ ID NO: 60) | |
| Bovine IgG1d | QTFTCNVAHPASSTKVDKAVDPTCKPSPCDCCPPPELPGGPSVFIFPPKPKDTLTISGTPEVTCVVVDVGHD |
| (CAA44699.1) | DPEV |
| (SEQ ID NO: 61) | |
| Minimally | QTYTCNVDHKPSNTKVDKAVDPTCKPSPCDCCPPPELPAGPSVFLFPPKPKDTLMISRTPEVTCVVVDVSHE |
| bovinized IgG2 | DPEV |
| (SEQ ID NO: 62) | |

TABLE 28
Minimally bovinized human IgG4

| | |
|---|---|
| Human IgG4 | ASTKGPSVFPLAPCSRSTSESTAALGCLVKDYFPEPVTVSWNSGALTSGVHTFPAVLQSSGLYSLSSVVTVP |
| (AAB59394.1) | SSSLGT |
| (SEQ ID NO: 63) | |
| Bovine IgG1d | ASTTAPKVYPLSSCCGDKSSSTVTLGCLVSSYMPEPVTVTWNSGALKSGVHTFPAVLQSSGLYSLSSMVTVP |
| (CAA44699.1) | GSTSG |
| (SEQ ID NO: 64) | |
| Minimally | ASTKGPSVFPLSSCCGDKSESTAALGCLVKDYFPEPVTVSWNSGALTSGVHTFPAVLQSSGLYSLSSVVTVP |
| bovinized IgG4 | SSSLGT |
| (SEQ ID NO: 65) | |
| Human IgG4 | KTYTCNVDHKPSNTKVDKRVESKYGP.PCPSCPAPEFLGGPSVFLFPPKPKDTLMISRTPEVTCVVVDVSQE |
| (AAB59394.1) | DPEV |
| (SEQ ID NO: 66) | |
| Bovine IgG1d | QTFTCNVAHPASSTKVDKAVDPTCKPSPCDCCPPPELPGGPSVFIFPPKPKDTLTISGTPEVTCVVVDVGHD |
| (CAA44699.1) | DPEV |
| (SEQ ID NO: 67) | |
| Minimally | KTYTCNVDHKPSNTKVDKAVDPTCKPSPCDCCPAPEFLGGPSVFLFPPKPKDTLMISRTPEVTCVVVDVSQE |
| bovinized IgG4 | DPEV |
| (SEQ ID NO: 68) | |

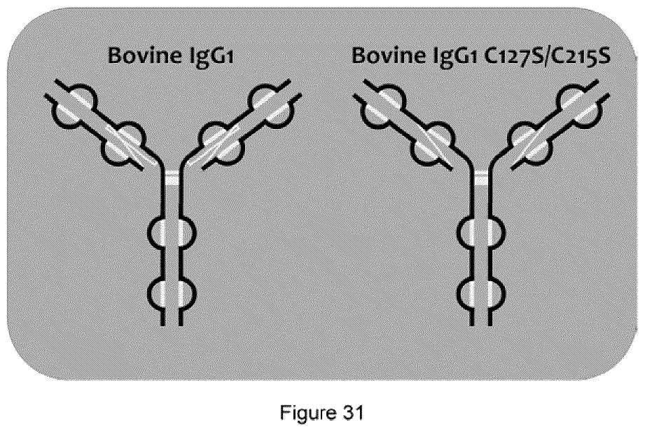

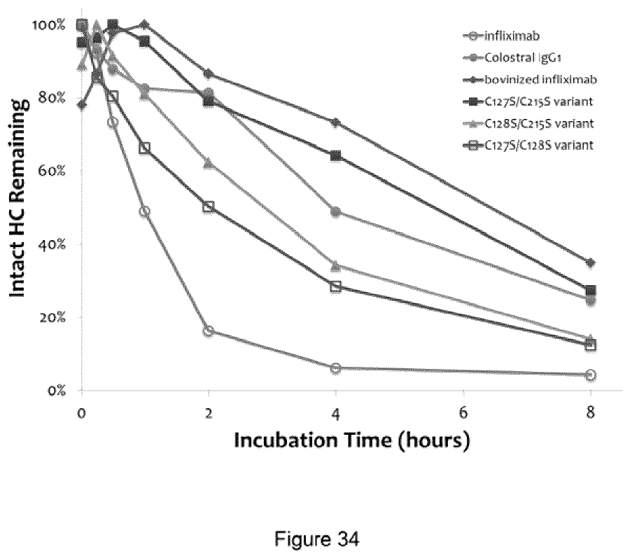

Also provided are bovinized antibodies comprising a bovine constant region, wherein the heavy chain comprises amino acid substitutions that remove particular disulfide bond linkages. Such antibodies are useful for testing the contribution of the CH1-hinge disulfide linkage and CH1-light chain disulfide linkage for conferring protease resistance to the parent antibody. As shown in FIG. 34, the bovinized infliximab construct is degraded more slowly by intestinal proteases than infliximab. The C127S/C215S, C128S/C215S and C127S/C128S variants (all of which are depicted in FIGS. 31-33) have intermediate stabilities between that of infliximab and bovinized infliximab. This supports the hypothesis that bovine IgG1 antibodies are stabilized by the combination of the CH1-hinge and CH1-LC disulfide bonds, as removing either disulfide bond destabilizes the molecule.

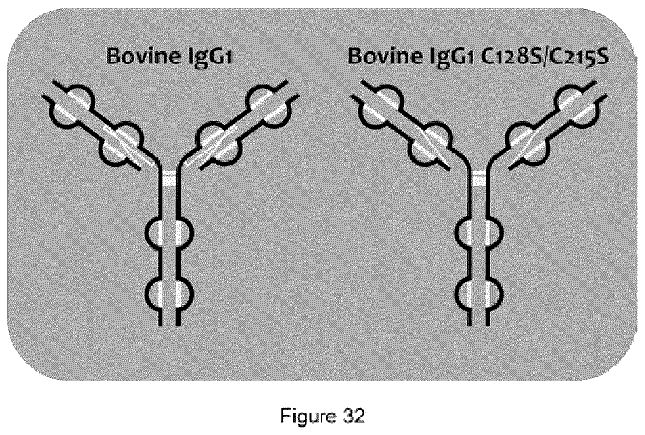

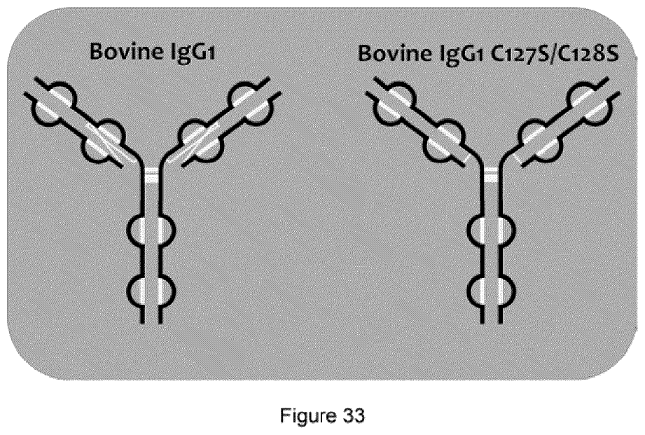

TABLE 29

| Description | Sequence |
|---|---|
| Bovinized infliximab | EVKLEESGGGLVQPGGSMKLSCVASGFIFSNHWMNWVRQSPEKGLEWVAEIRSKSINS |
| (C127S/C215S)-removes CH1- | ATHYAESVKGRFTISRDDSKSAVYLQMTDLRTEDTGVYYCSRNYYGSTYDYWGQGTTL |
| hinge linkage, resembles bovine | TVSSASTTAPKVYPLSSSCGDKSSSTVTLGCLVSSYMPEPVTVTWNSGALKSGVHTFP |
| IgG2 A2 allotype (see FIG. 31)- | AVLQSSGLYSLSSMVTVPGSTSGQTFTCNVAHPASSTKVDKAVDPTSKPSPCDCCPPP |
| heavy chain | ELPGGPSVFIFPPKPKDTLTISGTPEVTCVVVDVGHDDPEVKFSWFVDDVEVNTATTK |
| (SEQ ID NO: 69) | PREEQFNSTYRVVSALRIQHQDWTGGKEFKCKVHNEGLPAPIVRTISRTKGPAREPQV |
| | YVLAPPQEELSKSTVSLTCMVTSFYPDYIAVEWQRNGQPESEDKYGTTPPQLDADSSY |
| | FLYSKLRVDRNSWQEGDTYTCVVMHEALHNHYTQKSTSKSAGK |
| Bovinized infliximab | DILLTQSPAILSVSPGERVSFSCRASQFVGSSIHWYQQRINGSPRLLIKYASESMSGI |
| (C127S/C215S)-removes CH1- | PSRFSGSGSGTDFTLSINTVESEDIADYYCQQSHSWPFTFGSGTNLEVKRSDAEPSVF |
| hinge linkage, resembles bovine | LFKPSDEQLKTGTVSVVCLVNDFYPKDINVKWKVDGVTQSSSNFQNSFTDQDSKKSTY |
| IgG2 A2 allotype (see FIG. 31)- | SLSSILTLPSSEYQSHNAYTCEVSHKSLTTALVKSFSKNEC |
| light chain | |
| (SEQ ID NO: 70) | |
| Bovinized infliximab | EVKLEESGGGLVQPGGSMKLSCVASGFIFSNHWMNWVRQSPEKGLEWVAEIRSKSINS |
| (C128S/C215S)-removes CH1- | ATHYAESVKGRFTISRDDSKSAVYLQMTDLRTEDTGVYYCSRNYYGSTYDYWGQGTTL |
| hinge linkage, retains IgG1/CH1 | TVSSASTTAPKVYPLSSCSGDKSSSTVTLGCLVSSYMPEPVTVTWNSGALKSGVHTFP |
| loop structure (see FIG. 32)- | AVLQSSGLYSLSSMVTVPGSTSGQTFTCNVAHPASSTKVDKAVDPTSKPSPCDCCPPP |
| heavy chain | ELPGGPSVFIFPPKPKDTLTISGTPEVTCVVVDVGHDDPEVKFSWFVDDVEVNTATTK |
| (SEQ ID NO: 71) | PREEQFNSTYRVVSALRIQHQDWTGGKEFKCKVHNEGLPAPIVRTISRTKGPAREPQV |
| | YVLAPPQEELSKSTVSLTCMVTSFYPDYIAVEWQRNGQPESEDKYGTTPPQLDADSSY |
| | FLYSKLRVDRNSWQEGDTYTCVVMHEALHNHYTQKSTSKSAGK |
| Bovinized infliximab | DILLTQSPAILSVSPGERVSFSCRASQFVGSSIHWYQQRINGSPRLLIKYASESMSGI |
| (C128S/C215S)-removes CH1- | PSRFSGSGSGTDFTLSINTVESEDIADYYCQQSHSWPFTFGSGTNLEVKRSDAEPSVF |
| hinge linkage, retains IgG1/CH1 | LFKPSDEQLKTGTVSVVCLVNDFYPKDINVKWKVDGVTQSSSNFQNSFTDQDSKKSTY |
| loop structure (see FIG. 32)- | SLSSILTLPSSEYQSHNAYTCEVSHKSLTTALVKSFSKNEC |
| light chain | |
| (SEQ ID NO: 72) | |
| Bovinized infliximab | EVKLEESGGGLVQPGGSMKLSCVASGFIFSNHWMNWVRQSPEKGLEWVAEIRSKSINS |
| (C127S/C128S)-removes both | ATHYAESVKGRFTISRDDSKSAVYLQMTDLRTEDTGVYYCSRNYYGSTYDYWGQGTTL |
| CH1-LC and CH1-hinge linkages | TVSSASTTAPKVYPLSSSSGDKSSSTVTLGCLVSSYMPEPVTVTWNSGALKSGVHTFP |
| (see FIG. 33)-heavy chain | AVLQSSGLYSLSSMVTVPGSTSGQTFTCNVAHPASSTKVDKAVDPTCKPSPCDCCPPP |
| (SEQ ID NO: 73) | ELPGGPSVFIFPPKPKDTLTISGTPEVTCVVVDVGHDDPEVKFSWFVDDVEVNTATTK |
| | PREEQFNSTYRVVSALRIQHQDWTGGKEFKCKVHNEGLPAPIVRTISRTKGPAREPQV |
| | YVLAPPQEELSKSTVSLTCMVTSFYPDYIAVEWQRNGQPESEDKYGTTPPQLDADSSY |
| | FLYSKLRVDRNSWQEGDTYTCVVMHEALHNHYTQKSTSKSAGK |
| Bovinized infliximab | DILLTQSPAILSVSPGERVSFSCRASQFVGSSIHWYQQRINGSPRLLIKYASESMSGI |
| (C127S/C128S)-removes both | PSRFSGSGSGTDFTLSINTVESEDIADYYCQQSHSWPFTFGSGTNLEVKRSDAEPSVF |
| CH1-LC and CH1-hinge linkages | LFKPSDEQLKTGTVSVVCLVNDFYPKDINVKWKVDGVTQSSSNFQNSFTDQDSKKSTY |
| (see FIG. 33)-light chain | SLSSILTLPSSEYQSHNAYTCEVSHKSLTTALVKSFSKNEC |
| (SEQ ID NO: 74) | |

### Chimeric (Human/Ruminant and Human/Rabbit) Antibodies

Also provided herein are chimeric antibodies (e.g., chimeric infliximab) that fuse a parent variable region to a IgG1 constant region from other ruminants (e.g., sheep, goat) or rabbit.

The heavy chain sequences for the constructs below were designed by fusing the VH domain of infliximab with the CH1-hinge-CH2-CH3 sequences of the closest sequence match to the bovine IgG1d sequence used in Example 8. The light chain sequence contains the VL domain for infliximab fused to the CL domain with the closest sequence match to the infliximab CL domain, in order to maximize favorable interactions between the VL and CL domains of each construct.

TABLE 30
Combination of infliximab variable domain with sheep constant domain (Genbank:
CAA49451.1 for heavy chain, CAA38046.1 for light chain)

| | |
|---|---|
| Heavy Chain | EVKLEESGGGLVQPGGSMKLSCVASGFIFSNHWMNWVRQSPEKGLEWVAEIRSKSINSATHYAESVKGRFTI |
| (SEQ ID NO: 75) | SRDDSKSAVYLQMTDLRTEDTGVYYCSRNYYGSTYDYWGQGTTLTVSSASTTPPKVYPLTSCCGDTSSSIVT |
| | LGCLVSSYMPEPVTVTWNSGALTSGVHTFPAILQSSGLYSLSSVVTVPASTSGAQTFICNVAHPASSTKVDK |
| | RVEPGCPDPCKHCRCPPPELPGGPSVFIFPPKPKDTLTISGTPEVTCVVVDVGQDDPEVQFSWFVDNVEVRT |
| | ARTKPREEQFNSTFRVVSALPIQHQDWTGGKEFKCKVHNEALPAPIVRTISRTKGQAREPQVYVLAPPQEEL |
| | SKSTLSVTCLVTGFYPDYIAVEWQKNGQPESEDKYGTTTSQLDADGSYFLYSRLRVDKNSWQEGDTYACVVM |
| | HEALHNHYTQKSISKPPGK |
| Light Chain | DILLTQSPAILSVSPGERVSFSCRASQFVGSSIHWYQQRINGSPRLLIKYASESMSGIPSRFSGSGSGTDFT |
| (SEQ ID NO: 76) | LSINTVESEDIADYYCQQSHSWPFTFGSGTNLEVKRSDAQPSVFLFKPSEEQLRTGTVSVVCLVNDFYPKDI |
| | NVKVKVDGVTQNSNFQNSFTDQDSKKSTYSLSSTLTLSSSEYQSHNAYACEVSHKSLPTALVKSENKNEC |

TABLE 31
Combination of infliximab variable domain with goat constant domain (Genbank:
AAX45026.1 + CAA44699.1 for heavy chain, AAX45027.1 for light chain)

| | |
|---|---|
| Heavy Chain* | EVKLEESGGGLVQPGGSMKLSCVASGFIFSNHWMNWVRQSPEKGLEWVAEIRSKSINSATHYAESVKGRFTI |
| (SEQ ID NO: 77) | SRDDSKSAVYLQMTDLRTEDTGVYYCSRNYYGSTYDYWGQGTTLTVSSASTTPPKVYPLTSCCGDTSSSIVT |
| | LGCLVSSYMPEPVTVTWNSGALTSGVHTFPAVLQSSGLYSLSSMVTVPASTSGAQTFICNVAHPASSTKVDK |
| | RVEPGCKPSPCDCCPPPELPGGPSVFIFPPKPKDTLTISGTPEVTCVVVDVGHDDPEVKFSWFVDDVEVNTA |
| | TTKPREEQFNSTYRVVSALRIQHQDWTGGKEFKCKVHNEGLPAPIVRTISRTKGPAREPQVYVLAPPQEELS |
| | KSTVSLTCMVTSFYPDYIAVEWQRNGQPESEDKYGTTPPQLDADSSYFLYSKLRVDRNSWQEGDTYTCVVMH |
| | EALHNHYTQKSTSKSAGK |
| Light Chain | DILLTQSPAILSVSPGERVSFSCRASQFVGSSIHWYQQRINGSPRLLIKYASESMSGIPSRFSGSGSGTDFT |
| (SEQ ID NO: 78) | LSINTVESEDIADYYCQQSHSWPFTFGSGTNLEVKGQPKSAPSVTLFPPSTEELNANKATVVCLISDFYPGS |
| | VTVVWKADGSTINQNVKTTQASKQSNSKYAASSYLTLTGSEWKSKSSYSCEVTHEGSTVKKTVKPSEC |

*The goat CH1 and upper hinge sequence from AAX45026.1 was used, and fused to the lower hinge, CH2, and CH3 domains from the bovine IgGld sequence (CAA44699.1)

The goat CH1 and upper hinge sequence from AAX45026.1 was used, and fused to the lower hinge, CH2, and CH3 domains from the bovine IgG1d sequence (CAA44699.1)

TABLE 32
Combination of infliximab variable domain with rabbit constant domain (Genbank:
ABD64612.1 for heavy chain, 0507224A for light chain) (mAb#5)-rabbit IgG1 retains
bovine CH1 structure but with weaker hinge (equivalent to human IgG1 hinge)

| | |
|---|---|
| Heavy Chain | EVKLEESGGGLVQPGGSMKLSCVASGFIFSNHWMNWVRQSPEKGLEWVAEIRSKSINSATHYAESVKGRFTI |
| (SEQ ID NO: 79) | SRDDSKSAVYLQMTDLRTEDTGVYYCSRNYYGSTYDYWGQGTTLTVSSGSFKAPSVFPLAPCCGDTPSSTVT |
| | LGCLVKGYLPEPVTVTWNSGTLINGVRTFPSVRQSSGLYSLSSVVSVTSSSQPVTCNVAHPATNTKVDKTVA |
| | PSTCSKPTCPPPELLGGPSVFIFPPKPKDTLMISRTPEVTCVVVDVSQDDPEVQFTWYINNEQVRTARPPLR |
| | EQQFNSTIRVVSTLPIAHQDWLRGKEFKCKVHNKALPAPIEKTISKARGQPLEPKVYTMGPPREELSSRSVS |
| | LTCMINGFYPSDISVEWEKNGKAEDNYKTTPAVLDSDGSYFLYSKLSVPTSEWQRGDVFTCSVMHEALHNHY |
| | TQKSISRSPGK |
| Light Chain | DILLTQSPAILSVSPGERVSFSCRASQFVGSSIHWYQQRINGSPRLLIKYASESMSGIPSRFSGSGSGTDFT |
| (SEQ ID NO: 80) | LSINTVESEDIADYYCQQSHSWPFTFGSGTNLEVKGDPVAPTVLIFPPAADQVATGTVTIVCVANKYFPDVT |
| | VTWEVDGTTQTTGIENSKTPQDSADCTYNLSSTLTLTSTQYNSHKEYTCKVTQGTTSVVQSFNRGDC |

Bovinized Antibodies with Modifications to Modulate Biological Activity

Also provide are bovinized antibodies with amino acid substitutions that modulate biological activity. Specifically, shown below are amino acid substitutions to the bovine IgG1 hinge region for modulating FcR binding activity.

TABLE 33

| | |
|---|---|
| Human | CPPCPAPELLGGPSVFLFPPKPKDTLMISRTPE |
| (SEQ ID NO: 81) | VTCVVVDVSHED...VSNKALPAPIEK |
| Bovine | CDCCPPPELPGGPSVFIFPPKPKDTLTISGTPE |
| (SEQ ID NO: 82) | VTCVVVDVGHDD...VHNEGLPAPIVR |
| Mutant | CDCCPPPELLGGPSVFIFPPKPKDTLTISGTPE |
| (SEQ ID NO: 83) | VTCVVVDVSHDD...VHNEALPAPIVR |

Pepsin cleavage can be prevented by adding a bovine IgG3 hinge domain having a putative O-glycosylation site to the bovine IgG1 constant region.

TABLE 34

| | |
|---|---|
| bIgG3 | VDKAVTARRPVPTTPKTTIPPGKPTTPKSEVEK |
| (SEQ ID NO: 84) | TPCQ...CSKCPEP.L.GGLSVF |
| bIgG1 | VDKAV..........................DP |
| (SEQ ID NO: 85) | R.CKPSPCDCCPPPELPGGPSVF |
| Mutant | VDKAVTARRPVPTTPKTTIPPGKPTTPKSEVEP |
| (SEQ ID NO: 86) | R.CKPSPCDCCPPPELPGGPSVF |

Additional stabilization against proteolysis may be achieved by amino-acid substitutions in the lower hinge, as described in Table 35. Alternative hinge #1 contains a shorter sequence for the lower hinge, mimicking the bovine IgG2 sequence. Alternative hinge #2 has the same length as the bovine IgG1 hinge, but replaces the lower hinge sequence with proline residues, which inhibit degradation by most proteases. Alternative hinge #3 replaces the lower hinge sequence with alternating glycine and serine residues, which provide a hydrophilic, flexible sequence that maintains the conformational flexibility of the original sequence while removing potential proteolysis sites.

TABLE 35

| | |
|---|---|
| Bovine IgGld hinge | AVDPTCKPSPCDCCPPPELPGGPSVF |
| (SEQ ID NO: 87) | |
| Alternate hinge #1 | AVDPTCKPSPCDCC.VRE....PSVF |
| (SEQ ID NO: 88) | |
| Alternate hinge #2 | AVDPTCKPSPCDCCPPPPPPPPPSVF |
| (SEQ ID NO: 89) | |
| Alternate hinge #3 | AVDPTCKPSPCDCCPPPGSGSGPSVF |
| (SEQ ID NO: 90) | |

Recombinant IgG therapeutics are commonly purified by a Protein A affinity chromatography step. However, bovine IgG1 sequences do not bind to Protein A resins, due to differences in the amino-acid sequences of the CH2 and CH3 domains. Protein A binding may be reinstated by introducing amino-acid substitutions in the bovine CH2 and CH3 domains that re-create the binding interface between Protein A and human IgG1. Preferably, the substitutions made should avoid introducing strong binding to the human neonatal Fc receptor, as this would impact the biodistribution of the antibody in vivo. Exemplary amino acid substitutions to the bovine IgG1 sequence that may enhance Protein A binding, but not binding to human FcRn, include T252M, G255R, 0309L, T314L, G315N, or any combination thereof.

### Example 18—Expression of Bovinized Infliximab

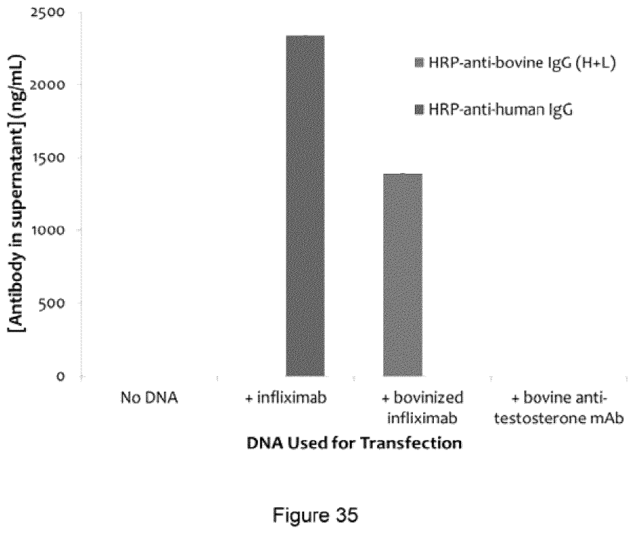

The expression of bovinized infliximab and infliximab expressed in HEK293 cells was compared. As shown in FIG. 35, the expression of bovinized infliximab and infliximab in HEK293 cells was comparable.

### Example 19—Large-Scale Production of Bovine IgG1 Monoclonal and Bovinized Antibodies

Bovine monoclonal and chimeric antibodies (e.g., bovine anti-TNF antibodies), such as those described in Examples 16 and 17, can be recombinantly produced in large-scale in various host organisms for use in research and clinical settings, as described below.

### Production in Yeast

Nucleic acids encoding the heavy and light chain of an antibody of interest (e.g., anti-TNF antibody) are cloned into appropriate yeast expression vectors. Yeast cells are grown in YEPD medium containing 2% glucose at 30° C. in a shaker for 4-6 hours to create a stock. 100 ml of the same media is inoculated with the stock and grown to an OD600 of 1.3-1.5. Cells are harvested by centrifugation, washed with water, and washed with sterile 1M sorbitol at 4° C. The cells are resuspended in 100 ul of 1M sorbitol, and 40 ul of cell suspension is added. Approximately 1 μg of plasmid DNA (two plasmids: one encoding the heavy chain and the other encoding the light chain, or alternatively one plasmid comprising sequences encoding both heavy and light chains) are added to the cells. The cells and plasmid DNA are incubated on ice for 5 min and transferred to 0.2 cm electroporation cuvettes. A Bio-Rad Gene Pulser (Richmond, Calif.) is used to perform the electroporation. Transformants are selected on YNB media lacking uracil.

A preculture is grown in 100 ml YNB media (ura-, leu⁺)+2% glucose for 24 hrs at 30° C., followed by inoculation in 200 ml fermentation media, (ura-, leu-)+2% glucose, at 0.5 OD. Cultures are grown and harvested and samples taken at various time points and induced with 2% galactose. Cells are separated from the culture media by centrifugation, and culture supernatants are concentrated with Centricon 30 filters by ultrafiltration and used for SDS-PAGE analysis. Antibody purity and yield can be detected by running concentrated supernatant on SDS-PAGE.

The procedure above can be up-scaled using 2-10 L fermenters for large-scale fermentations.

### Production in Aspergillus

Nucleic acids encoding the heavy and light chain of an antibody of interest (e.g., anti-TNF antibody) are cloned into separate expression vectors with selectable markers known in the art (e.g., pGAMpR, or those described for filamentous fungi in Sambrook et al., 1989 and Ausubel FM et al., 1989).

Aspergillus niger or Aspergillus niger var. awamori strain (which has a deletion in the gene encoding a major secreted aspartyl protease) are used as host cells. Transformations and large-scale fermentations are carried out as described in Ward et al. (Appl Environ Microb 2004; 70:2567-576). Yields using the protocol described in Ward et al. can reach 0.2-0.9 g/L.

Briefly, cells are incubated at 37° C. in flasks on a shaker for 2 days. 2-day-old medium is transferred and incubated at 30° C. on a shaker for 5 days. Transformants producing high levels of antibody and balanced amounts of light and heavy chains are identified by SDS-PAGE or ELISAs, and subsequently used for large-scale production.

Selected transformants are grown in large (i.e., 14-liter) fermentors. After 48 to 60 hours of incubation at 30° C. on a shaker, cultures are transferred and incubated overnight on a shaker at 30° C. Antibodies in culture supernatants are quantified by ELISA. Antibodies are purified from the culture supernatant by removal of the fungal cells by filtration through a cellulose pad and filtration of the supernatant. Hydrophobic charge induction chromatography (HCIC) and high-performance liquid chromatography (HPLC) are used to purify the supernatant. Antibody purity and yield are detected by running concentrated supernatant on SDS-PAGE.

### Production in Mammalian Cells

Mammalian cells (e.g., Chinese hamster ovary (CHO) cells, and NS0 murine myeloma cells) are used for large-scale production of antibodies. Nucleic acids encoding the heavy and light chain of an antibody of interest (e.g., anti-TNF antibody) are cloned into appropriate mammalian expression vectors and transfected into cells. If necessary, adherent cell lines are adapted to suspension culture formats for large-scale production (see, e.g., Sinacore et al., Biotechnol Bioeng 1996; 52: 518-28).

Cultures are grown in media (e.g., DMEM) without serum at 37° C. Stable cell line clones are selected using methods known in the art, such as through metabolic markers, including methotrexate (MTX). To identify high-producing clones, cells are separated from the culture media by centrifugation, and culture supernatants are concentrated by ultrafiltration and used for SDS-PAGE analysis. Antibody purity and yield can be detected by running concentrated supernatant on SDS-PAGE. The top yielding clones are selected for further evaluation in large-scale bioreactors (e.g., 2,000 liters) to determine the final production clone.

### Production in Insect Cells

Insect cells (e.g., Sf-9 cells) can be used in the large-scale production of antibodies of interest. Nucleic acids encoding the heavy and light chain of an antibody of interest (e.g., anti-TNF antibody) are cloned into a baculovirus expression vector. Cells are cultured at 25° C.-30° C. in Grace's Supplemented (TNM-FH) medium and grown overnight. Cells are infected with the baculovirus and harvested at various time points to measure antibody production.

To measure antibody production, supernatants are clarified before further processing with cartridge membranes or ultrafiltration membranes. Membrane filtration or chromatographic techniques are used to remove baculoviruses from the supernatant. The resulting supernatants are concentrated by ultrafiltration and used for SDS-PAGE analysis. Antibody purity and yield can be detected by running concentrated supernatant on SDS-PAGE.

### Example 20—Antibody Purification

The antibodies described herein may be purified by several procedures commonly used in the production of recombinant antibodies, including precipitation by ammonium sulfate, antigen affinity chromatography, thioaffinity chromatography, or binding to bacterial proteins that have high affinity for mammalian immunoglobulins. Beads coupled to recombinant Streptococcus Protein G have been used to purify the IgG molecules described herein. A 5 mL Protein G Sepharose HiTrap column (GE Healthcare cat #17-0405-01) was equilibrated with 1×PBS (10 mM sodium phosphate, 150 mM sodium chloride, pH 7.4). The sample was loaded onto the column, washed with PBS, and the antibody was eluted with 5 column volumes of 100 mM glycine, pH 2.7. The fraction collection tubes contained 100 μL 1 M TrisCl, pH 9 to neutralize the eluate fractions. The elution fractions were pooled and concentrated with an Amicon 10k MWCO spin filter to purify the antibodies.

### Example 21—Treatment of DSS and DNBS Mouse Colitis Model with Bovine Recombinant Monoclonal IgG1 or Bovinized Anti-TNF Antibodies

C57BL/6 mice are used to induce artificial colitis, essentially as described in WO2012/058769.

For dextran sulfate sodium (DSS)-induced colitis, DSS is added to drinking water for a final concentration of 5% (wt/volume) for a total of 5 days. Mice are administered a bovinized or fully bovine recombinant monoclonal anti-TNF antibody (selected and produced as described in Examples 14-16) or an isotype control antibody (e.g., IgG1 antibody) for 6 days starting one day prior to exposure to DSS. Onset of colitis is assessed using the disease activity index (DAI). The DAI is a combined score of weight loss, stool consistency, and fecal bleeding. The scoring system is as follows: weight loss: 0, no loss; 1, 1-5%; 2, 5-10%; 3, 10-20%; 4, 20%+; stool: 0, normal; 2, loose stool; 4, diarrhea; and bleeding: 0, no blood, 2, Hemoccult positive; and 4, gross blood (blood around anus). DAI is measured on all 5 days of DSS treatment.

For DNBS-induced colitis, C57BL/6 mice are anesthetized, and a 10-cm long tubing attached to a tuberculin syringe is intrarectally inserted 3.5 cm into the colon and in order to induce colitis. 100 μL of 5 mg of DNBS solution dissolved in 50% ethanol is administered and left for 3 days. Controls receive vehicle (50% ethanol) for the same duration.

To assess macroscopic damage, mice are sacrificed 5 days post-DSS or 3 days post-DNBS administration, the abdominal cavity is opened, and observations on colonic distension, fluid content, hyperemia, and erythema are recorded. The colon is removed and macroscopic damage assessed on the full section of the colon. Macroscopic scores are performed using previously described scoring systems for DSS colitis (Cooper et al. Lab Invest 1993; 69:238-49) and DNBS (Khan et al., Infect Immun 2002; 70:5931-5937).

For colonic histology assessments, colon segments are fixed in formalin and embedded in paraffin, followed by sectioning and staining with hematoxylin and eosin. Colonic damage is blindly scored based on the DSS colitis scoring system described above. The scoring system considers loss of architecture (0, normal-3, severe), cellular infiltration (0, normal-3, severe), muscle thickening (0, normal-3, severe), goblet cell depletion (0, absent; 1, present), crypt abscess (0, absent; 1, present).

MPO activity measurements are carried out as described in Khan et al. (Infect Immun 2002; 70:5931-7). Colonic tissue samples are homogenized in ice-cold 50 mmol/L potassium phosphate buffer (pH=6.0) containing 0.5% hexadecyl trimethyl ammonium bromide, homogenates are centrifuged, the supernatant is removed, and an aliquot is added to a solution containing potassium phosphate buffer, O-dianisidine, and hydrogen peroxide. Absorbance is measured at 450 mm using a spectrophotometer. MPO activity is expressed in units per milligram of wet tissue, where 1 unit is the quantity of enzyme able to convert 1 μmol of hydrogen peroxide to water in 1 minute at room temperature.

DAI scores, histology, and MPO activity are compared between treatment groups.

### Example 22—Treatment of Mouse Model of TNBS-Induced Colitis with Oral Fully Bovine Recombinant Monoclonal IgG1 or Bovinized Anti-TNF Antibodies

C57BL/6 mice ((8-9 weeks old) Charles River Laboratories, Wilmington, Mass.) are administered 0.1 mL TNBS (trinitrobenzene sulfonate) (4 mg) in 50% ethanol intrarectally. The TNBS model is a well-accepted model of inflammatory bowel disease, Control animals are dosed with ethanol alone. Twelve animals are used in TNBS-treated group and eight animals in each of the other groups. Animals are dosed with 0.05 mg, 0.015 mg, or 0.005 mg of a bovinized or fully bovine recombinant monoclonal anti-TNF IgG1 antibody (selected and produced as described in Examples 14-16), 0.015 mg of an isotype control antibody (e.g., IgG1 antibody), or saline twice per day by oral gavage in 0.1 mL. All samples contain 0.2 mg ovalbumin as an excipient. Antibody is administered from day −1 to day 3.

Each mouse is analyzed using video endoscopy, under isoflurane anesthesia, on day 5 just prior to being sacrificed. During each endoscopic procedure still images as well as video are recorded to evaluate the extent of colitis and the response to treatment. Colitis severity is scored by a blinded observer using a 0-4 scale (0-normal; 1=loss of vascularity; 2=loss of vascularity and friability; 3=friability and erosions; 4=ulcerations and bleeding). Differences between groups are analyzed.

### Example 23—Treatment of Adoptive-Transfer Model of Chronic Colitis with Fully Bovine Recombinant Monoclonal IgG1 or Bovinized Anti-TNF Antibodies

Chronic CD45Rb^{high} transfer colitis is induced essentially as described in U.S. Pat. No. 8,119,401. Briefly, BALB/c splenocytes are first enriched for CD4⁺ cells by red cell lysis and negative selection using the following rat anti-mouse mAbs to B220 (clone RA3-6B2), Mac-1 (clone M1/70), and CD8α MAb-stained cells are removed in a magnetic field using sheep anti rat IgG coated magnetic beads (Dynal, Hamburg, Germany). The resulting CD4 enriched cells are stained with cychrome (Cy)-conjugated CD4 and fluorescein isothiocyanate (FITC)-conjugated CD45RB mAbs. Subpopulations of CD4 cells are generated by two color sorting on the FACS sorter (Becton Dickinson).

C.B-17 SCID mice are injected intraperitoneally with sorted CD4⁺ cell subpopulations in PBS. To induce colitis, CD45RB^{high} CD4⁺ cells (1−4×10⁵) are transferred to the SCID mice. Starting on the day of transfer, the mice are administered 0.1 mg of a bovinized or fully bovine recombinant monoclonal anti-TNF IgG1 antibody (selected and produced as described in Examples 14-16), or an isotype control antibody (e.g., IgG1) in saline buffer once a day. Antibodies are formulated in 10 mg bovine serum albumin.

Mice are weighed twice a week and wasting disease is determined by percentage of weight loss from baseline body weight. Peripheral blood is drawn at different time points by retro-orbital sinus puncture for plasma cytokine measurements.

Mice are scarified approximately 4-6 weeks after transfer, colons are isolated and fixed in 4% formalin, embedded in paraffin, sectioned, and stained with hematoxylin and eosin for histological grading. Inflammation is scored by an experienced pathologist blinded to treatment allocation on a scale of 0-4, representing no change to severe changes, as described in Powrie et al. (Induction of inflammatory bowel disease in immunodeficient mice by depletion of regulatory T cells. In: Coico R, ed. Current protocols in immunology. Vol. 4: John Wiley & Sons, Inc., 1999:153.1-0.3.0).

### Example 24—Antibody Efficacy

Efficacy of the antibodies described herein can be tested in vitro using the ELISA, TNF neutralization, and pancreatin digestion assays described in Examples 2, 3, and 11. The in vivo efficacy of the antibodies can be tested using animal models relevant to the target of interest. For instance, for bovinized and fully bovine recombinant monoclonal anti-TNF IgG1 antibodies, the animal model systems described in Examples 21-23 can be used. The efficacy of these antibodies can be determined in parallel with that of AVX-470 and/or infliximab and compared. Antibodies having the desired properties (e.g., ELISA binding, TNF neutralizing potency, and pancreatin resistance) are selected.

### Example 25—Antibody Biodistribution

Bovinized and fully bovine recombinant monoclonal antibodies described herein are conjugated to a detectable label (e.g., a radioactive label such as ^{99m}Tc) using standard methods (see, e.g., D'Alessandria et al., Q J Nucl Med Mol Imaging 2007; 51:334-42) in order evaluate their biodistribution in mice. Briefly, mice are orally dosed with the labeled antibodies, and sacrificed at different time points after antibody dosing (e.g., 30 min, 1, 2, 4, 6, 8, 12, and 24 hours after injection). Serum is collected after sacrifice. The gastrointestinal tract is removed, lavaged with a fixed volume of saline, sectioned, and weighed, and, if the label is radioactive, assessed for radioactivity. Organ activity is expressed as a percentage of administered activity (% AD)/gram tissue or/mL of serum or gut lavage fluid. The biodistribution of the antibodies described herein can be determined in parallel with a known antibody. For instance, for bovinized and fully bovine recombinant monoclonal anti-TNF IgG1 antibodies, the biodistribution of these antibodies can be determined and compared in parallel with that of AVX-470 and/or infliximab.

The patent and scientific literature referred to herein establishes the knowledge that is available to those with skill in the art. All United States patents and published or unpublished United States patent applications cited herein are incorporated by reference. AR published foreign patents and patent applications cited herein are hereby incorporated by reference. AU other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

### Equivalents

Those skilled in the art will recognize, or be able to ascertain using no more than routine experimentation, many equivalents of the specific embodiments disclosed herein.

We claim:

1. An isolated recombinant monoclonal antibody comprising (a) heavy and light chain CDRs from a non-bovine antibody and (b) a constant region comprising all or a portion of a bovine IgG1 constant region.

2. The antibody of claim 1, wherein the non-bovine antibody is a human antibody.

3. The antibody of claim 1, wherein the constant region comprises all or a portion of a bovine IgG1 hinge region.

4. The antibody of claim 1, wherein the constant region comprises at least one of
(i) all or a portion of a bovine IgG1 CH1 domain;
(ii) all or a portion of a bovine IgG1 CH2 domain; and
(iii) all or a portion of a bovine IgG1 CH3 domain.

5. The antibody of claim 1, wherein the antibody comprises one or more of the following features found in bovine IgG1:
a) a disulfide bond linking the N-terminus of the CH1 domain to the N-terminus of the hinge region,
b) a disulfide bond linking the N-terminus of the CH1 domain to the C-terminus of the light chain, and
c) a cluster of three disulfide bonds linking the hinge regions of two heavy chains together.

6. The antibody of claim 1, wherein the bovine IgG1 constant region is selected from any of SEQ ID NOs: 1-3.

7. The antibody of claim 1, wherein the constant region further comprises a portion of a human constant region.

8. The antibody of claim 7, wherein the human constant region is an IgG1 or IgG2.

9. An isolated recombinant monoclonal human antibody, wherein the constant region of the antibody comprises one or more of the following features found in bovine IgG1:
a) a disulfide bond linking the N-terminus of the CH1 domain to the N-terminus of the hinge region,
b) a disulfide bond linking the N-terminus of the CH1 domain to the C-terminus of the light chain, and
c) a cluster of three disulfide bonds linking the hinge regions of two heavy chains together.

10. The antibody of claim 9, wherein the antibody binds to a biological antigen.

11. The antibody of claim 10, wherein the antigen is TNF-α.

12. The antibody of claim 11, wherein the antibody comprises the heavy and light chain variable region sequences of infliximab.

13. The antibody of claim 1, wherein the antibody retains antigen binding after protease digestion.

14. The antibody of claim 1, wherein the antibody comprises constant region comprising one or more of the following substitutions (Kabat numbering): threonine at position 252 is substituted with methionine; glycine at position 255 is substituted with arginine; glutamine at position 309 is substituted with leucine; threonine at position 314 is substituted with leucine; and glycine at position 315 is substituted with asparagine.

15. A recombinant fully bovine monoclonal IgG1 antibody.

16. A nucleic acid encoding the antibody of claim 1.

17. An expression vector comprising the nucleic acid of claim 16.

18. A host cell comprising the nucleic acid of claim 16.

19. A pharmaceutical composition comprising the antibody of claim 1 and a carrier and, optionally, a preservative, the composition being optionally formulated for oral administration.

20. The composition of claim 19 which is lyophilized.

21. A method of treating a disease of the digestive tract comprising orally administering to an individual in need thereof the antibody of claim 1.

22. The method of claim 21, wherein the disease is selected from the group consisting of ulcerative colitis and Crohn's disease.

23. A method of producing the antibody of claim 1, comprising expressing the antibody in a host cell and isolating the antibody from the cell.
